SEQUENCE LISTING (40 sequences)

SEQ ID NO: 1 - DNA, 100 bp, Artificial Sequence
  Synthetic Polynucleotide
ttcattatccatcctccatcgccacgatagttcatggcgataggtagaatagcaatgaacgattatccctatcaagcattctgactgagcattgctcaca

SEQ ID NO: 2 - DNA, 327 bp, Artificial Sequence
  Synthetic Polynucleotide
tgtggcttttatgaaaatcacacagtgatcacaaattttaaacagagcacaaaatgctgcctcgaaatgagggcgggaaaataaggttatcagccttgttttctccctcattacttgaaggatatgaagctaaaacccttttttataaagcatttgtccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagaggggagcacattgatg

SEQ ID NO: 3 - DNA, 306 bp, Artificial Sequence
  Synthetic Polynucleotide
gcttagatcaggtgattgccctttgtttatgagggtgttgtaatccatgtcgttgttgcatttgtaagggcaacacctcagcctgcaggcaggcactgaagataccaaagggtagttcagattacacggtcacctggaaagggggccattttactttttatcgccgctggcggtgcaaagttcacaaagttgtcttacgaaggttgtaaggtaaaacttatcgatttgataatggaaacgcattagccgaatcggcaaaaattggttaccttacatctcatcgaaaacacggaggaagtatagatg

SEQ ID NO: 4 - DNA, 144 bp, Artificial Sequence
  Synthetic Polynucleotide
cacagctaacaccacgtcgtccctatctgctgccctaggtctatgagtggttgctggataactttacgggcatgcataaggctcgtataatatattcagggagaccacaacggtttccctctacaaataattttgtttaacttt

SEQ ID NO: 5 - DNA, 144 bp, Artificial Sequence
  Synthetic Polynucleotide
cacagctaacaccacgtcgtccctatctgctgccctaggtctatgagtggttgctggataactttacgggcatgcataaggctcgtaggctatattcagggagaccacaacggtttccctctacaaataattttgtttaacttt

SEQ ID NO: 6 - DNA, 74 bp, Artificial Sequence
  Synthetic Polynucleotide
tccctatcagtgatagagattgacatccctatcagtgatagagatactgagcacatcagcaggacgcactgacc

SEQ ID NO: 7 - DNA, 15 bp, Artificial Sequence
  Synthetic Polynucleotide
attaaagaggagaaa

SEQ ID NO: 8 - DNA, 15 bp, Artificial Sequence
  Synthetic Polynucleotide
ggtttcctgtgtgaa

SEQ ID NO: 9 - DNA, 11 bp, Artificial Sequence
  Synthetic Polynucleotide
tcacacaggac

SEQ ID NO: 10 - DNA, 14 bp, Artificial Sequence
  Synthetic Polynucleotide
tcacacaggaaacc

SEQ ID NO: 11 - DNA, 26 bp, Artificial Sequence
  Synthetic Polynucleotide
tagtttacgaggtaaggaggtttaag

SEQ ID NO: 12 - DNA, 29 bp, Artificial Sequence
  Synthetic Polynucleotide
actaacaataaagtataaggaggtctaca

SEQ ID NO: 13 - DNA, 28 bp, Artificial Sequence
  Synthetic Polynucleotide
acgacataaaattaataaggaggtaaaa

SEQ ID NO: 14 - DNA, 25 bp, Artificial Sequence
  Synthetic Polynucleotide
tgagtacgtagggaggaggttaaaa

SEQ ID NO: 15 - DNA, 75 bp, Artificial Sequence
  Synthetic Polynucleotide
ttaaacaaaattatttgtagaggctgtttcgtcctcacggactcatcagaccggaaagcacatccggtgacagct

SEQ ID NO: 16 - DNA, 918 bp, Artificial Sequence
  Synthetic Polynucleotide
atgaatattcgtgatcttgagtacctggtggcattggctgaacaccgccattttcggcgtgcggcagattcctgccacgttagccagccgacgcttagcgggcaaattcgtaagctggaagatgagctgggcgtgatgttgctggagcggaccagccgtaaagtgttgttcacccaggcgggaatgctgctggtggatcaggcgcgtaccgtgctgcgtgaggtgaaagtccttaaagagatggcaagccagcagggcgagacgatgtccggaccgctgcacattggtttgattcccacagttggaccgtacctgctaccgcatattatccctatgctgcaccagacctttccaaagctggaaatgtatctgcatgaagcacagacccaccagttactggcgcaactggacagcggcaaactcgattgcgtgatcctcgcgctggtgaaagagagcgaagcattcattgaagtgccgttgtttgatgagccaatgttgctggctatctatgaagatcacccgtgggcgaaccgcgaatgcgtaccgatggccgatctggcaggggaaaaactgctgatgctggaagatggtcactgtttgcgcgatcaggcaatgggtttctgttttgaagccggggcggatgaagatacacacttccgcgcgaccagcctggaaactctgcgcaacatggtggcggcaggtagcgggatcactttactgccagcgctggctgtgccgccggagcgcaaacgcgatggggttgtttatctgccgtgcattaagccggaaccacgccgcactattggcctggtttatcgtcctggctcaccgctgcgcagccgctatgagcagctggcagaggccatccgcgcaagaatggatggccatttcgataaagttttaaaacaggcggtttaa

SEQ ID NO: 17 - DNA, 711 bp, Artificial Sequence
  Synthetic Polynucleotide
atggtgagcaagggcgaagaagataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 18 - DNA, 714 bp, Artificial Sequence
  Synthetic Polynucleotide
atggtttccaaaggtgaagaactgattaaagaaaacatgcacatgaagctgtacatggaaggtactgtaaacaaccaccacttcaaatgtaccagcgaaggcgaaggcaaaccgtatgagggcacccaaaccatgcgtatcaaagttgtggaaggcggtccgctgccgtttgcattcgacatcctggcgaccagcttcatgtacggcagcaaaaccttcatcaaccacactcaaggtatcccggattttttcaaacagagcttcccggagggctttacctgggaacgcgttacgacgtatgaagatggtggcgtcctgaccgctacgcaggacacgtctctgcaggatggctgtctgatctataacgttaaaattcgtggtgttaatttcccgagcaacggcccggttatgcagaaaaaaacgctgggctgggaagcatccaccgaaatgctgtacccggctgacggcggcctggaaggccgttctgatatggcgctgaaactggttggtggcggccacctgatctgtaacctgaaaactacttaccgcagcaaaaaaccggctaaaaacctgaaaatgccgggcgtatattatgtcgaccgccgtctggaacgtatcaaagaagcggacaaagaaacctatgtcgaacagcatgaagtggcagtggcacgctattgcgatctgccttccaaactgggccacaaactgaactaa

SEQ ID NO: 19 - DNA, 717 bp, Artificial Sequence
  Synthetic Polynucleotide
atgtctaaaggtgaagaattattcactggtgttgtcccaattttggttgaattagatggtgatgttaatggtcacaaattttctgtctccggtgaaggtgaaggtgatgctacgtacggtaaattgaccttaaaatttatttgtactactggtaaattgccagttccatggccaaccttagtaactactttgagccatggtgttcaatgtttttctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaacttcaactctcacaatatatacatcatggctgacaaacaaaagaatggtatcaaagtgaacttcaaaattagacacaacattgaagatggttctgttcaattagctgaccattatcaacaaaatactccaattggtgatggtccagtcttgttaccagacaaccattacttatccacccaatcagccttatccaaagatccaaacgaaaagagagaccacatggtcctgttagaatttaggactgctgctggtattacccatggtatggatgaattgtacaaataa

SEQ ID NO: 20 - DNA, 717 bp, Artificial Sequence
  Synthetic Polynucleotide
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgatctctacaaataa

SEQ ID NO: 21 - DNA, 1500 bp, Artificial Sequence
  Synthetic Polynucleotide
atgagagccctggtagtcatccgcctgtcccgcgtcaccgatgctacgacttcaccggagcgtcagctggagtcttgccagcagctctgcgcccagcgcggctgggacgtcgtcggggtagcggaggatctggacgtctccggggcggtcgatccgttcgaccggaagcgcagaccgaacctggcccggtggctagcgttcgaggagcaaccgttcgacgtgatcgtggcgtaccgggtagaccggttgacccgatcgatccggcatctgcagcagctggtccactgggccgaggaccacaagaagctggtcgtctccgcgaccgaagcgcacttcgatacgacgacgccgtttgcggcggtcgtcatcgcgcttatgggaacggtggcgcagatggaattagaagcgatcaaagagcggaaccgttcggctgcgcatttcaatatccgcgccgggaaataccgaggatccctgccgccgtggggatacctgcctacgcgcgtggacggggagtggcggctggtgccggaccctgtgcagcgagagcgcatcctcgaggtgtatcaccgcgtcgtcgacaaccacgagccgctgcacctggtggcccacgacctgaaccggcgtggtgtcctgtcgccgaaggactacttcgcgcagctgcaaggccgcgagccgcagggccgggagtggtcggctaccgcgctgaagcgatcgatgatctccgaggcgatgctcgggtacgcgactctgaacggtaagaccgtccgagacgacgacggagccccgctggtgcgggctgagccgatcctgacccgtgagcagctggaggcgctgcgcgccgagctcgtgaagacctcccgggcgaagcccgcggtgtctaccccgtcgctgctgctgcgggtgttgttctgtgcggtgtgcggggagcccgcgtacaagttcgccgggggaggacgtaagcacccgcgctaccgctgccgctcgatggggttcccgaagcactgcgggaacggcacggtggcgatggccgagtgggacgcgttctgcgaggagcaggtgctggatctgctcggggacgcggagcgtctggagaaagtctgggtagccggctcggactccgcggtcgaactcgcggaggtgaacgcggagctggtggacctgacgtcgctgatcggctccccggcctaccgggccggctctccgcagcgagaagcactggatgcccgtattgcggcgctggccgcgcggcaagaggagctggagggtctagaggctcgcccgtctggctgggagtggcgcgagaccgggcagcggttcggggactggtggcgggagcaggacaccgcggcaaagaacacctggcttcggtcgatgaacgttcggctgacgttcgacgtccgcggcgggctgactcgcacgatcgacttcggggatctgcaggagtacgagcagcatctcaggctcggcagcgtggtcgaacggctacacaccgggatgtcg

SEQ ID NO: 22 - DNA, 1839 bp, Artificial Sequence
  Synthetic Polynucleotide
atgacacaaggggttgtgaccggggtggacacgtacgcgggtgcttacgaccgtcagtcgcgcgagcgcgagaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcg

SEQ ID NO: 23 - DNA, 1455 bp, Artificial Sequence
  Synthetic Polynucleotide
atgactaagaaagtagcaatctatacacgagtatccactactaaccaagcagaggaaggcttctcaattgatgagcaaattgaccgtttaacaaaatatgctgaagcaatggggtggcaagtatctgatacttatactgatgctggtttttcaggggccaaacttgaacgcccagcaatgcaaagattaatcaacgatatcgagaataaagcttttgatacagttcttgtatataagctagaccgcctttcacgtagtgtaagagatactctttatcttgttaaggatgtgttcacaaaaaataaaatagactttatctcgcttaatgaaagtattgatacttcttctgctatgggtagcttgtttctcactattctttctgcaattaatgagtttgaaagagagaatataaaagaacgcatgactatgggtaaactagggcgagcgaaatctggtaagtctatgatgtggactaagacagcttttgggtattaccacaacagaaagacaggtatattagaaattgttcctttacaagctacaatagttgaacaaatattcactgattatttatcaggaatatcacttacaaaattaagagataaactcaatgaatctggacacatcggtaaagatataccgtggtcttatcgtaccctaagacaaacacttgataatccagtttactgtggttatatcaaatttaaggacagcctatttgaaggtatgcacaaaccaattatcccttatgagacttatttaaaagttcaaaaagagctagaagaaagacaacagcagacttatgaaagaaataacaaccctagacctttccaagctaaatatatgctgtcagggatggcaaggtgcggttactgtggagcacctttaaaaattgttcttggccacaaaagaaaagatggaagccgcactatgaaatatcactgtgcaaatagatttcctcgaaaaacaaaaggaattacagtatataatgacaataaaaagtgtgattcaggaacttatgatttaagtaatttagaaaatactgttattgacaacctgattggatttcaagaaaataatgactccttattgaaaattatcaatggcaacaaccaacctattcttgatacttcgtcatttaaaaagcaaatttcacagatcgataaaaaaatacaaaagaactctgatttgtacctaaatgattttatcactatggatgagttgaaagatcgtactgattcccttcaggctgagaaaaagctgcttaaagctaagattagcgaaaataaatttaatgactctactgatgtttttgagttagttaaaactcagttgggctcaattccgattaatgaactatcatatgataataaaaagaaaatcgtcaacaaccttgtatcaaaggttgatgttactgctgataatgtagatatcatatttaaattccaactcgct

SEQ ID NO: 24 - DNA, 49 bp, Artificial Sequence
  Synthetic Polynucleotide
cggccggcttgtcgacgacggcggtctccgtcgtcaggatcatccgggc

SEQ ID NO: 25 - DNA, 58 bp, Artificial Sequence
  Synthetic Polynucleotide
gtcgtggtttgtctggtcaaccaccgcggtctcagtggtgtacggtacaaaccccgac

SEQ ID NO: 26 - DNA, 45 bp, Artificial Sequence
  Synthetic Polynucleotide
tgcgggtgccagggcgtgcccttgggctccccgggcgcgtactcc

SEQ ID NO: 27 - DNA, 42 bp, Artificial Sequence
  Synthetic Polynucleotide
gtgccccaactggggtaacctttgagttctctcagttggggg

SEQ ID NO: 28 - DNA, 53 bp, Artificial Sequence
  Synthetic Polynucleotide
atgccaacacaattaacatctcaatcaaggtaaatgctttttgctttttttgc

SEQ ID NO: 29 - DNA, 50 bp, Artificial Sequence
  Synthetic Polynucleotide
gcgagtttttatttcgtttatttcaattaaggtaactaaaaaactccttt

SEQ ID NO: 30 - DNA, 90 bp, Artificial Sequence
  Synthetic Polynucleotide
ttcagccaaaaaacttaagaccgccggtcttgtccactaccttgcagtaatgcggtggacaggatcggcggttttcttttctcttctcaa

SEQ ID NO: 31 - DNA, 57 bp, Artificial Sequence
  Synthetic Polynucleotide
ggaaacacagaaaaaagcccgcacctgacagtgcgggctttttttttcgaccaaagg

SEQ ID NO: 32 - DNA, 36 bp, Artificial Sequence
  Synthetic Polynucleotide
gcagcaaacgacgaaaactacgctgcagcagtttag

SEQ ID NO: 33 - DNA, 105 bp, Artificial Sequence
  Synthetic Polynucleotide
ggcatcaaataaaacgaaaggctcagtcgaaagactgggcctttcgttttatctgttgtttgtcggtgaacgctctcctgagtaggacaaatccgccgccctaga

SEQ ID NO: 34 - DNA, 106 bp, Artificial Sequence
  Synthetic Polynucleotide
gcttggactcctgttgatagatccagtaatgacctcagaactccatctggatttgttcagaacgctcggttgccgccgggcgttttttattggtgagaatccaagc

SEQ ID NO: 35 - DNA, 758 bp, Artificial Sequence
  Synthetic Polynucleotide
cggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtt

SEQ ID NO: 36 - DNA, 2221 bp, Artificial Sequence
  Synthetic Polynucleotide
gtacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagt

SEQ ID NO: 37 - DNA, 1009 bp, Artificial Sequence
  Synthetic Polynucleotide
aggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttac

SEQ ID NO: 38 - DNA, 950 bp, Artificial Sequence
  Synthetic Polynucleotide
tcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattcc

SEQ ID NO: 39 - DNA, 797 bp, Artificial Sequence
  Synthetic Polynucleotide
cgatatctggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttgatatcg

SEQ ID NO: 40 - DNA, 615 bp, Artificial Sequence
  Synthetic Polynucleotide
gggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccag

(19) United States
(12) Patent Application Publication
(10) Pub. No.: US 2017/0373700 A1
(43) Pub. Date: Dec. 28, 2017

(54) ANALOG TO DIGITAL COMPUTATIONS IN BIOLOGICAL SYSTEMS

(71) Applicant: Massachusetts Institute of Technology, Cambridge, MA (US)

(72) Inventors: Timothy Kuan-Ta Lu, Cambridge, MA (US); Jacob Rosenblum, Cambridge, MA (US); Gianluca Selvaggio, Rimini (IT)

(73) Assignee: Massachusetts Institute of Technology, Cambridge, MA (US)

(21) Appl. No.: 15/535,846

(22) PCT Filed: Dec. 22, 2015
(86) PCT No.: PCT/US2015/067381

§ 371 Date: Jun. 14, 2017

Related U.S. Application Data
(60) Provisional application No. 62/095,318, filed on Dec. 22, 2014.

(51) Int. Cl. H03M 1/36; C12N 5/10; C12N 15/63; G06N 3/00

(52) U.S. Cl. CPC H03M 1/361; G06N 3/002; C12N 5/10; C12N 15/63

(57) ABSTRACT

Aspects of the present disclosure relate to analog signal processing circuits and methods for cellular computation.

### RELATED APPLICATIONS

This application is a national stage filing under 35 U.S.C. §371 of international application number PCT/US2015/067381, filed Dec. 22, 2015, which was published under PCT Article 21(2) in English and claims the benefit under 35 U.S.C. §119(e) of U.S. provisional application No. 62/095,318, filed Dec. 22, 2014, each of which is incorporated by reference herein in its entirety.

### FEDERALLY SPONSORED RESEARCH

This invention was made with government support under Grant No. HDTRA1-14-1-0007 awarded by the Defense Threat Reduction Agency and Grant No. N00014-11-1-0725 awarded by the Office of Naval Research. The government has certain rights in the invention.

### FIELD

Aspects of the present disclosure relate to the field of biosynthetic engineering.

### BACKGROUND

Biology uses a mixed signal approach to understand an environment and implement an appropriate response. This mixed signal approach is often a combination of analog and digital signal processing. To date, most work in gene circuit design has been focused on digital signal processing.

### SUMMARY

Provided herein, in some aspects, are gene circuits and methods for analog signal processing. One of the aims of synthetic biology is to leverage biochemistry to implement computation (e.g., cellular computation). For complex computations, it is beneficial to have sensors that can measure the concentration of molecules of interest over a wide-range of concentrations. Previously, such wide-range sensors have altered the expression of genes on a continuous spectrum proportionally to the concentration of the molecule of interest. The present disclosure demonstrates that promoters with different affinities to a wide-range sensor can be used to control the expression of genes discretely at different thresholds concentrations of the molecule of interest.

Living cells implement complex computations upon the continuous environmental signals that they encounter. These computations involve both analog and digital-like processing of signals to give rise to complex developmental programs, context-dependent behaviors, and homeostatic activities. Embodiments of the present disclosure provide integrated analog and digital computation to implement complex hybrid synthetic genetic programs in living cells. Herein, in some embodiments, is a framework for building comparator gene circuits (also referred to herein as biological analog signal processing circuits, or analog-to-digital converters (ADCs)) to digitize analog inputs based on different thresholds. Comparators can be predictably composed together to build more complex circuits such as bandpass filters, ternary logic systems, and multi-level ADCs. Additionally, these analog-to-digital circuits can interface with other digital gene circuits to enable concentration-dependent logic in which intermediate input levels, rather than extreme ones, control the output. This hybrid computational paradigm enables new industrial, diagnostic, and therapeutic applications with engineered cells.

In some aspects, the disclosure provides a biological analog signal processing circuit comprising (a) a first promoter operably linked to a nucleic acid encoding a regulatory protein responsive to an input signal; (b) a second promoter responsive to the regulatory protein and operably linked to a nucleic acid encoding a first output protein, wherein activity of the second promoter is altered when bound by the regulatory protein; and, (c) an output molecule flanked by a first set of regulatory sequences, wherein the first set of regulatory sequences interacts with the first output protein to operably link the output molecule to a third promoter.

In some aspects the disclosure provides a biological analog signal processing circuit comprising (a) a first promoter operably linked to a nucleic acid encoding a regulatory protein responsive to an input signal; (b) a second promoter responsive to the regulatory protein and operably linked to a nucleic acid encoding a first output protein, wherein activity of the second promoter is altered when bound by the regulatory protein; and (c) an output molecule operably linked to a third promoter, wherein the output molecule or the third promoter is flanked by a first set of regulatory sequences, wherein the first set of regulatory sequences interacts with the first output protein to unlink the output molecule from the third promoter.

In some embodiments, the circuit further comprises: (d) a fourth promoter responsive to the regulatory protein and operably linked to a nucleic acid encoding a second output protein, wherein activity of the fourth promoter is altered when bound by the regulatory protein; and, (e) a second output molecule flanked by a second set of regulatory sequences, wherein the second set of regulatory sequences interacts with the second output protein to operably link the second output molecule to a fifth promoter.

In some embodiments, the promoter of (a) as described above is a constitutively-active promoter. In some embodiments, the regulatory protein is oxyR.

In some embodiments, the promoter of (b) and/or (d) as described above comprises a modification that alters the binding affinity of a transcription factor or RNA polymerase for the promoter of (b) and/or (d), relative to a similar unmodified promoter. In some embodiments, the promoter of (b) and/or (d) is a naturally occurring promoter. In some embodiments, the promoters of (b) and (d) are bound by the same transcription factor with different affinities.

In some embodiments, the modification is a nucleic acid mutation.

In some embodiments, (a), (b) and (c) as described above are on a vector. In some embodiments, (a), (b), (c) and (d) as described above are on a vector. In some embodiments, (a) and (b) are on a single vector. In some embodiments, (a), (b) and (d) are on a single vector. In some embodiments, the vector is a low copy plasmid, a medium copy plasmid or a high copy plasmid. In some embodiments, (c) and/or (e) is on a bacterial artificial chromosome (BAC).

In some embodiments, (b) and/or (d) further comprises a sequence element that regulates production of the first output protein and is located between the second promoter and the nucleic acid encoding the first output protein. In some embodiments, the sequence element regulates transcription or translation of the output protein. In some embodiments, the sequence element is a ribosomal binding site. In some embodiments, the sequence element is a modified ribosomal binding site comprising a modification that alters the binding affinity of a ribosome for the modified ribosomal binding site, relative to a similar unmodified ribosomal binding site.

In some embodiments, the promoter of (b) and/or (d) is an oxyR promoter that comprises a modification that alters the binding affinity of a transcription factor or RNA polymerase for oxyR promoter of (b), relative to a similar unmodified promoter. In some embodiments, the promoter of (b) and/or (d) is a naturally occurring oxyR promoter. In some embodiments, the promoters of (b) and (d) are naturally occurring oxyR promoters that have different affinities for oxyR protein.

In some embodiments, the first output protein of (b) is a recombinase and the first set of regulatory sequences of (c) is recombinase recognition sites. In some embodiments, the second output protein of (d) is a recombinase and the second set of regulatory sequences of (e) is recombinase recognition sites. In some embodiments, the first output molecule of (c) is a fluorescent protein. In some embodiments, the second output molecule of (e) is a fluorescent protein.

In some aspects, the disclosure provides a cell or cell lysate comprising the circuit of any one of the preceding claims. In some embodiments, the cell is a bacterial cell. In some embodiments, the bacterial cell is an Escherichia coli cell. In some embodiments, the cell or cell lysate of any one of the preceding claims further comprising the input signal. In some embodiments, the input signal modulates activity of the regulatory protein. In some embodiments, the input signal activates activity of the regulatory protein. In some embodiments, the input signal is a chemical input signal. In some embodiments, the chemical input signal is hydrogen peroxide.

In some aspects, the disclosure relates to a biological bandpass filter comprising: (a) a first promoter operably linked to a nucleic acid encoding a regulatory protein responsive to an input signal; (b) a second promoter responsive to the regulatory protein and operably linked to a nucleic acid encoding a first bandpass protein, wherein activity of the second promoter is altered when bound by the regulatory protein; (c) a third promoter responsive to the regulatory protein and operably linked to a nucleic acid encoding a second bandpass protein, wherein the third promoter is not the second promoter and wherein activity of the third promoter is altered when bound by the regulatory protein; and (d) a first output molecule flanked by a first set of regulatory sequences, wherein the first set of regulatory sequences interacts with the first bandpass protein to operably link the first output molecule to a fourth promoter, wherein the fourth promoter is flanked by a second set of regulatory sequences, and wherein the second set of regulatory sequences interacts with the second bandpass protein to unlink the first output molecule from the fourth promoter.

In some aspects, the disclosure relates to a biological bandpass filter comprising: (a) a first promoter operably linked to a nucleic acid encoding a regulatory protein responsive to an input signal; (b) a second promoter responsive to the regulatory protein and operably linked to a nucleic acid encoding a first bandpass protein, wherein activity of the second promoter is altered when bound by the regulatory protein; (c) a third promoter responsive to the regulatory protein and operably linked to a nucleic acid encoding a second bandpass protein, wherein the third promoter is not the second promoter and wherein activity of the third promoter is altered when bound by the regulatory protein; and (d) a first output molecule operably linked to a fourth promoter, flanked by a first set of regulatory sequences, wherein the first set of regulatory sequences interacts with the first bandpass protein to unlink the first output molecule from the fourth promoter, wherein the first set of regulatory sequences is flanked by a second set of regulatory sequences, and wherein the second set of regulatory sequences interacts with the second bandpass protein to operably link the first output molecule to the fourth promoter.

In some embodiments, the promoter of (a) is a constitutively-active promoter. In some embodiments, the regulatory protein is oxyR.

In some embodiments, the second promoter of (b) and/or the third promoter of (c) as described above comprises a modification that alters the binding affinity of a transcription factor or RNA polymerase for the second promoter of (b) and or the third promoter of (c) relative to a similar unmodified promoter. In some embodiments, the modification is a nucleic acid mutation.

In some embodiments, (a), (b) and (c) as described above are on a vector. In some embodiments, (a), (b) and (c) as described above on the same vector. In some embodiments, the vector is a low copy plasmid, a medium copy plasmid or a high copy plasmid. In some embodiments, (d) is on a bacterial artificial chromosome (BAC).

In some embodiments, (b) further comprises a sequence element that regulates production of the first bandpass protein and is located between the second promoter and the nucleic acid encoding the first bandpass protein. In some embodiments, (c) further comprises a sequence element that regulates production of the second bandpass protein and is located between the third promoter and the nucleic acid encoding the second bandpass protein. In some embodiments, the sequence element regulates transcription or translation of the first bandpass protein and/or second bandpass protein. In some embodiments, the sequence element is a ribosomal binding site. In some embodiments, the sequence element is a modified ribosomal binding site comprising a modification that alters the binding affinity of a ribosome for the modified ribosomal binding site, relative to a similar unmodified ribosomal binding site.

In some embodiments, the promoter of (b) is an oxyR promoter that comprises a modification that alters the binding affinity of a transcription factor or RNA polymerase for oxyR promoter of (b), relative to a similar unmodified promoter. In some embodiments, the promoter of (c) is an oxyR promoter that comprises a modification that alters the binding affinity of a transcription factor or RNA polymerase for oxyR promoter of (c), relative to a similar unmodified promoter.

In some embodiments, the first bandpass protein of (b) is a recombinase. In some embodiments, the second bandpass protein of (c) is a recombinase. In some embodiments, the first set of regulatory sequences and/or the second set of regulatory sequences of (d) are recombinase recognition sites. In some embodiments, the first output protein of (d) is a fluorescent protein.

In some aspects, the instant disclosure relates to a cell or cell lysate comprising the circuit of any one of the preceding claims. In some embodiments, the cell is a bacterial cell.

In some embodiments, the bacterial cell is an Escherichia coli cell. In some embodiments, the cell or cell lysate further comprises the input signal. In some embodiments, the input signal modulates activity of the regulatory protein. In some embodiments, the input signal activates activity of the regulatory protein. In some embodiments, the input signal is a chemical input signal. In some embodiments, the chemical input signal is hydrogen peroxide.

In some aspects, the disclosure relates to a biological analog signal processing circuit comprising: (a) a first promoter operably linked to a nucleic acid encoding a regulatory protein responsive to an input signal; (b) a second promoter responsive to the regulatory protein and operably linked to a nucleic acid encoding a first bandpass protein, wherein activity of the second promoter is altered when bound by the regulatory protein; (c) a third promoter responsive to the regulatory protein and operably linked to a nucleic acid encoding a second bandpass protein, wherein the third promoter is not the second promoter and wherein activity of the third promoter is altered when bound by the regulatory protein; (d) a fourth promoter responsive to the regulatory protein and operably linked to a nucleic acid encoding a third bandpass protein, wherein the fourth promoter is not the first and not the second promoter and wherein activity of the fourth promoter is altered when bound by the regulatory protein; (e) a first output molecule flanked by a first set of regulatory sequences, wherein the first set of regulatory sequences interacts with the first bandpass protein to operably link the first output molecule to a fifth promoter, wherein the fifth promoter and a sixth promoter are flanked by a second set of regulatory sequences, wherein the second set of regulatory sequences interacts with the second bandpass protein to unlink the fifth promoter from the first output molecule and to operably link the fifth promoter to a second output molecule; and, (f) a third set of regulatory sequences flanking the sixth promoter, wherein the third set of regulatory sequences interacts with the third bandpass protein to operably link the sixth promoter to the first output molecule without unlinking the fifth promoter from the second output molecule.

In some embodiments, the promoter of (a) as described in the paragraph above is a constitutively-active promoter. In some embodiments, the regulatory protein is oxyR.

In some embodiments, the second promoter of (b) and/or the third promoter of (c) and/or the fourth promoter of (d) as described in the paragraph above comprises a modification that alters the binding affinity of a transcription factor or RNA polymerase for the promoter of (b) and/or (c) and/or (d), relative to a similar unmodified promoter. In some embodiments, the modification is a nucleic acid mutation.

In some embodiments, (a), (b), (c) and/or (d) are on a vector. In some embodiments, (a), (b), (c) and/or (d) are on the same vector. In some embodiments, the vector is a low copy plasmid, a medium copy plasmid or a high copy plasmid. In some embodiments, (e) and (f) are on a bacterial artificial chromosome (BAC). In some embodiments, (e) and (f) are on a single bacterial artificial chromosome (BAC).

In some embodiments, (b) further comprises a sequence element that regulates production of the first bandpass protein and is located between the second promoter and the nucleic acid encoding the first bandpass protein. In some embodiments, (c) further comprises a sequence element that regulates production of the second bandpass protein and is located between the third promoter and the nucleic acid encoding the second bandpass protein. In some embodiments, (d) further comprises a sequence element that regulates production of the third bandpass protein and is located between the fourth promoter and the nucleic acid encoding the third bandpass protein. In some embodiments, the sequence element regulates transcription or translation of the first bandpass protein and/or the second bandpass protein and/or the third bandpass protein. In some embodiments, the sequence element is a ribosomal binding site. In some embodiments, the sequence element is a modified ribosomal binding site comprising a modification that alters the binding affinity of a ribosome for the modified ribosomal binding site, relative to a similar unmodified ribosomal binding site.

In some embodiments, the promoter of (b) is an oxyR promoter that comprises a modification that alters the binding affinity of a transcription factor or RNA polymerase for oxyR promoter of (b), relative to a similar unmodified promoter. In some embodiments, the promoter of (c) is an oxyR promoter that comprises a modification that alters the binding affinity of a transcription factor or RNA polymerase for oxyR promoter of (c), relative to a similar unmodified promoter. In some embodiments, the promoter of (d) is an oxyR promoter that comprises a modification that alters the binding affinity of a transcription factor or RNA polymerase for oxyR promoter of (d), relative to a similar unmodified promoter.

In some embodiments, the first bandpass protein of (b) is a recombinase. In some embodiments, the second bandpass protein of (c) is a recombinase. In some embodiments, the third bandpass protein of (d) is a recombinase. In some embodiments, the first set of regulatory sequences and/or the second set of regulatory sequences of (d) and/or the third set of regulatory sequences of (e) are recombinase recognition sites. In some embodiments, the first output molecule of (e) is a fluorescent protein.

In some aspects, the disclosure relates to a cell or cell lysate comprising the circuit of any one of the preceding claims. In some embodiments, the disclosure relates to a cell or cell lysate comprising a combination of at least two circuits of any one of the preceding claims. In some embodiments, the cell is a bacterial cell. In some embodiments, the bacterial cell is an Escherichia coli cell. In some embodiments, the cell or cell lysate further comprises the input signal. In some embodiments, the input signal modulates activity of the regulatory protein. In some embodiments, the input signal activates activity of the regulatory protein. In some embodiments, the input signal is a chemical input signal. In some embodiments, the chemical input signal is hydrogen peroxide.

In some aspects, the disclosure relates to a method of analog signal processing in cells, comprising: providing a cell or cell lysate that comprises the circuit of any one of the preceding claims; and contacting the cell with an input signal that modulates the regulatory protein. In some embodiments, the method further comprises contacting the cell or cell lysate with different concentrations of the input signal. In some embodiments, the method comprises detecting in the cell or cell lysate an expression level of the output molecule and, optionally, quantifying levels of the output molecule. In some embodiments of the method, the cell is a bacterial cell. In some embodiments, the bacterial cell is an Escherichia coli cell. In some embodiments, the output molecule is a reporter molecule, an enzyme, a therapeutic molecule or a nucleic acid molecule.

### BRIEF DESCRIPTION OF DRAWINGS

The accompanying drawings are not intended to be drawn to scale. For purposes of clarity, not every component may be labeled in every drawing.

FIG. 1 shows a schematic of an example of a framework for engineering complex, robust cellular computation.

FIG. 2 shows a schematic of one embodiment of a biological analog signal processing circuit. In the circuit, a transcription factor (oxyR) senses a wide continuous range of input (H₂O₂) and is not saturated. It may be expressed via positive feedback, negative feedback, or open-loop expression; the transcription factor binds promoters with different affinities (promoter 1 vs. promoter 2), and so it activates these promoters at different concentrations of the input; the promoters express recombinases. Some of the recombinases have different translation rates (RBS1 vs. RBS2), causing them to “turn ON” at different concentrations of the input despite being expressed from the same promoter; the recombinases (e.g. BxbI, PhiC31, TP901) flip ON or OFF output genes (e.g. GFP, RFP, BFP); the output genes are located on a BAC to minimize their copy number in the cell, and therefore the number of states that the output can take (closer to a digital zero or one). In another embodiment, these output genes could be placed in the genome to further minimize the number of states that output genes can take. Different behaviors, for example a three-concentration classifier, a canonical analog to digital converter and/or a bandpass filter can result by rearranging the recombinase sites and output genes.

FIGS. 3A-3B show one example of a biological analog signal processing circuit component. FIG. 3A shows a schematic of the component. The oxyR transcription factor is constitutively produced. It senses H₂O₂ and actives promoters (oxySp or katGp) with different affinities. The promoters control the transcription of a recombinase (BxbI or PhiC31). The recombinases then “flip” on GFP expression. FIG. 3B presents data tracking the percent of cells in a population that have turned ON GFP expression in response to different concentrations of H₂O₂. By placing these promoters in control of recombinases, digital switches (with regard to input H₂O₂ at different concentrations) are produced.

FIGS. 4A-4B show one example of a biological analog signal processing circuit component. In this example, the translation rates of the recombinases are altered via the use of different ribosomal binding sites (RBS). FIG. 4A shows a schematic of the component. The katGp promoter is used to drive translation of both recombinases (BxbI and PhiC31). Two different RBS (RBS2 and RBS1, respectively) are paired with the recombinases. Depending on the RBS strength, the translation efficiency of the recombinase is different. As a result, the concentration of H₂O₂ necessary for activation is different. These recombinases then “flip” on GFP expression. FIG. 4B presents data tracking the percent of cells in a population that have turned ON GFP expression in response to different concentrations of H₂O₂. By changing the translation rate of the recombinase from the same promoter, digital switches (with regards to input H₂O₂ at different concentrations) are produced.

FIG. 5 provides data demonstrating that combinations of different promoters and RBS can be used to tune the biological analog processing circuit. In this example, the promoter/RBS combinations of the circuit have been tuned to produce similar expression levels with regard to input H₂O₂ at different concentrations.

FIGS. 6A-6D show one example of a biological analog signal processing circuit used as a three-concentration classifier. In this design, the oxyR transcription factor is constitutively produced. It senses H₂O₂ and actives promoters, here denoted as promoter 1 and promoter 2 (for example, oxySp and katGp) with different affinities. These promoters each control the transcription of a recombinase (for example, BxbI, PhiC31, or TP901). Depending on the RBS strength, the translation efficiency of the recombinase is different. As a result, the combination of different promoters and translation strengths alters the concentration of H₂O₂ necessary for activation for the recombinases. These recombinases then “flip” on the expression of their outputs: GFP, RFP, or BFP. FIG. 6A shows “State 0” of this model; at a low concentration of H₂O₂, there is no expression of any output molecule. FIG. 6B shows “State 1” of this model; at a first concentration, promoter 1 is activated by oxyR and BxbI is expressed. BxbI “flips ON” expression of GFP but not the other output molecule. FIG. 6C shows “State 2” of this model; at a second concentration, promoter 2 is activated by oxyR and PhiC31 is expressed. PhiC31 “flips ON” expression of RFP. Note that GFP is still expressed in this state but BFP is not expressed. FIG. 6D shows “State 3” of this model; at a third concentration, promoter 3 is activated by oxyR and TP901 is expressed. TP901 “flips ON” expression of BFP. Note that all three output molecules are expressed in “State 3”.

FIG. 7 shows one example of a biological analog signal processing circuit used as a bandpass filter. In this example, the oxyR transcription factor is constitutively produced. It senses H₂O₂ and actives promoters, here denoted as promoter 1 and promoter 2 (for example oxySp and katGp), with different affinities. The promoters control the transcription of a recombinase (for example, BxbI and PhiC31). Depending on the RBS strength, the translation efficiency of the recombinase is different. As a result, the combination of different promoters and translation strengths alters the concentration of H₂O₂ necessary for activation for the recombinases. These recombinases then “flip” different things. In this example, BxbI flips GFP “ON”, and PhiC31 flips the promoter “OFF”. The BxbI recombinase has a lower threshold [H₂O₂], and therefore GFP is turned “ON” at medium concentrations of H₂O₂. However, at higher [H₂O₂], PhiC31 is turned “ON”, and it flips the promoter “OFF”, thus turning GFP “OFF”. The cumulative effect is a bandpass filter for intermediate concentrations of hydrogen peroxide.

FIGS. 8A-8D show one example of the design of a biological two-bit analog to digital converter. In this example, the oxyR transcription factor is constitutively produced. It senses H₂O₂ and actives promoters, here denoted as promoter 1 and promoter 2 (for example oxySp and katGp), with different affinities. The promoters control the transcription of a recombinase (for example, BxbI, PhiC31 and TP901). Depending on the RBS strength, the translation efficiency of the recombinase is different. As a result, the combination of different promoters and translation strengths alters the concentration of H₂O₂ necessary for activation for the recombinases. These recombinases then “flip” different components. FIG. 8A shows “State 0” of this model; at a low concentration of H₂O₂, there is no expression of any output molecules (GFP and mCherry). FIG. 8B shows “State 1” of this model; In “State 1”, BxbI flips GFP “ON”. FIG. 8C shows “State 2” of this model; at a higher [H₂O₂], PhiC31 flips GFP “OFF” and simultaneously flips mCherry “ON” by flipping the promoters of both GFP and mCherry. FIG. 8D shows “State 3” of this model; at a higher [H₂O₂] than “State 2”, TP901 flips the promoter linked to GFP “ON”. At the highest [H₂O₂], both GFP and mCherry are expressed.

FIG. 9A and FIG. 9B show one example of a biological analog signal processing circuit component. In this example, the transcription rates of the recombinases are altered via the use of different versions of the same promoter. FIG. 9A shows a schematic of the component, with the left schematic showing the use of oxySp promoter and the right schematic showing the use of oxySpM promoter. The RBS in each circuit is the same (0030). The promoters controls the transcription of a recombinase (Bxbi). Depending on the promoter strength, the transcription efficiency of the recombinase is different. As a result, the concentration of H₂O₂ necessary for activation is different, as is shown in FIG. 9B. These recombinases then “flip” on GFP expression. FIG. 9B shows that using the oxySp promoter, full activation of GFP expression occurs at less than 1.0 μM H₂O₂ (upper curve). In contrast, at less than 1.0 μM H₂O₂, the oxySpM promoter drives less than 20% activation of GFP expression, whereas full activation of GFP expression requires almost 10 μM H₂O₂ (lower curve).

FIGS. 10A-10C show an example of an analog H₂O₂-sensor. FIG. 10A shows OxyR constitutively expressed from a low-copy plasmid (LCP), which activates transcription of gfp from the oxySp promoter on the same LCP in response to H₂O₂. FIG. 10B shows the geometric mean of GFP expression at different concentrations of H₂O₂ measured three hours after induction. The line is a Hill function fit to the data. The errors (standard error of the mean) are derived from flow cytometry experiments of three biological replicates, each of which involved n>30,000 gated events. FIG. 10C shows representative flow cytometry histograms for the analog circuit shown at in FIG. 10A at different H₂O₂ concentrations. GFP is measured with FITC. GFP expression is continuously activated with increasing H₂O₂ over at least two orders of magnitude of the input.

FIGS. 11A-11D shows an overview of an example of a comparator. FIG. 11A shows that at low input concentrations, the transcription factor gene (tf) is constitutively expressed, but the TF is not activated to a significant level. Consequently, the invertase gene is not expressed. FIG. 11B shows that at medium input concentrations, the TF is activated (TF bound to Input), but it is below the concentration needed for significant expression of the invertase gene. FIG. 11C show that at high input concentrations, the concentration of activated TF is sufficient to activate expression of the invertase from a specific promoter (pTF). The Invertase (Inv) binds to the invertase sites (triangles) and inverts the DNA between the sites. This results in the expression of the output gene by the upstream promoter (arrow), leading to output expression. FIG. 11D shows a genetic comparator diagram. It is composed of the threshold module (pTF+RBS), the digitization module (Invertase) and the output module (Output). An input activates a sensor (such as a transcription factor), and this transcription factor activates the expression of an invertase at an input threshold (0) defined by the affinity of the invertase promoter for the activated transcription factor and by the translation strength of the invertase as defined by its RBS. When the invertase is expressed, the output is switched ON.

FIGS. 12A-12G show an example of digitization of an analog input by inverting target DNA on a medium-copy plasmid (MCP) versus a bacterial artificial chromosome (BAC). FIG. 12A shows OxyR is constitutively expressed from a LCP and activating transcription of bxb1 from the oxySp* promoter on the same LCP in response to H₂O₂. Bxb1 inverts the gfp expression construct on a BAC or MCP, turning on gfp expression by pairing it with an upstream proD promoter. FIG. 12B shows the percent of GFP positive cells at different H₂O₂ concentrations as measured by flow cytometry. The BAC (circles) and MCP (squares) have similar transfer functions. However, the MCP exhibits a higher basal level of cells that are GFP positive. The errors (standard deviation) are derived from flow cytometry experiments of three biological replicates, each of which involved n>30,000 gated events. FIG. 12C shows representative flow cytometry histograms for the BAC circuit shown in FIG. 12A at different H₂O₂ concentrations. GFP is measured with FITC. The GFP-positive cells maintain a consistent level of GFP fluorescence even with increased H₂O₂, indicating a homogeneous population. FIG. 12D shows representative flow cytometry histograms for the MCP circuit shown in FIG. 12A at different H₂O₂ concentrations. The GFP-positive cells demonstrate increasing levels of GFP fluorescence with increased H₂O₂, indicating that there are multiple heterogeneous subpopulations. FIG. 12E shows that the % of GFP positive cells vs. concentration of H₂O₂ (circles) for the BAC circuit from FIG. 12A is fit to a transfer function and plotted on the left y-axis. The geometric mean of the GFP positive cells in FIG. 12C relative to the minimum geometric mean of the GFP positive cells in the same experiment vs. concentration of H₂O₂ (black squares) is plotted on the right y-axis and adjacent points are directly connected by straight lines (black line). The geometric mean does not considerably increase with H₂O₂, indicating that GFP positive cells in FIG. 12C constitute one population even at different levels of the input. FIG. 12F shows the % of GFP positive cells vs. concentration of H₂O₂ (circles) for the MCP circuit from FIG. 12A is fit to a transfer function and plotted on the left y-axis. The geometric mean of the GFP positive cells in FIG. 12D relative to the minimum geometric mean of the GFP positive cells in the same experiment vs. concentration of H₂O₂ (black squares) is plotted on the right y-axis and adjacent points are directly connected by straight lines (black line). The geometric mean increases considerably with H₂O₂, indicating that GFP positive cells in FIG. 12D take on multiple populations with different H₂O₂ levels. FIG. 12G shows digitization of the input by the comparator circuit. The percent of GFP positive cells at different H₂O₂ concentrations as measured by flow cytometry for the BAC comparator circuit (red circles) is plotted on the left axis (same data as black squares in FIG. 12B). For comparison, we have also plotted the geometric mean of GFP expression at different concentrations of H₂O₂ (black squares) on the right axis (same data as circles in FIG. 10B). The five-highest tested concentrations of H₂O₂ continuously increase GFP expression from the inducible promoter but do not increase the percent of GFP positive cells from a comparator.

FIGS. 13A-13C show an example of a feedforward cascade involving a recombinase-invertible trans-acting transcriptional element on a BAC. FIG. 13A shows OxyR is constitutively expressed from a LCP and activates transcription of bxb1 from the oxySp* promoter on the same LCP in response to H₂O₂. Bxb1 inverts the tetR expression cassette on a BAC, turning on TetR expression by pairing it with the proD promoter. TetR represses gfp expression from pLtetO on a MCP. FIG. 13B shows the percent of GFP positive cells at different H₂O₂ concentrations as measured by flow cytometry. The transfer function has a narrow switching range. The errors (standard deviation) are derived from flow cytometry experiments of three biological replicates, each of which involved n>30,000 gated events. FIG. 13C shows representative flow cytometry histograms for the circuit shown at in FIG. 13A at different H₂O₂ concentrations. GFP is measured with FITC. The GFP-positive cells fall into one population.

FIGS. 14A-14E show an example of amplifying BAC output with Copy Control. A BAC that also has an origin of replication that can be activated by a plasmid replication factor integrated into the genome of EPI300 E. coli under inducible control by Copy Control (CC) reagent was used. FIG. 14A shows cells were first incubated with different concentrations of H₂O₂ to induce GFP expression. Cells were then washed and diluted into fresh media with CC. Copy Control (CC) induces trfA expression from the pBAD promoter via activation of AraC, which are both expressed from the EPI300 chromosome. TrfA amplifies the BAC from 1-2 copies per cell to a high copy plasmid (HCP) at ˜100 copies per cell38. FIG. 14B shows flow cytometry histograms for GFP expression from the BAC with CC and without CC at 121 μM H₂O₂. Copy Control (CC) amplifies GFP expression at least 63.5× as measured by the geometric means of the populations. FIG. 14C shows the transfer functions for the BAC with CC (black line, black squares) and without CC (line, circles) are nearly identical. The errors (standard deviation) are derived from flow cytometry experiments of three biological replicates, each of which involved n>30,000 gated events. FIG. 14D shows representative flow cytometry histograms for the BAC at different concentrations of H₂O₂ without CC for the data in FIG. 14C. FIG. 14E shows representative flow cytometry histograms for the BAC at different concentrations of H₂O₂ with CC for the data in FIG. 14C. The experiments in FIGS. 14B and 14D were measured with the same FITC voltage on the flow cytometer, and FIG. 14E was measured with a different, lower FITC voltage on the flow cytometer because GFP expression from the BAC+CC was greater than the measurable fluorescence at the higher FITC voltage (as can be seen in the +CC data in FIG. 14B).

FIGS. 15A-15B show examples of genetic comparators with different activation thresholds. FIG. 15A shows the low-threshold H₂O₂ comparator circuit. OxyR is constitutively expressed from a low-copy plasmid (LCP) and activates transcription of bxb1 recombinase from either the oxySp or oxySp* promoter on the same LCP in response to H₂O₂. Bxb1 translation is altered by the strength of the ribosome binding site (RBS). Bxb1 inverts the gfp expression cassette located between inversely oriented attB and attP sites (triangles) on a bacterial artificial chromosome (BAC), thus turning on GFP expression. The gfp cassette has a ribozyme sequence for cleaving the 5′ untranslated region of an mRNA transcript (RiboJ)⁵⁸, a computationally designed RBS⁵⁹, the gfp coding sequence, and a transcriptional terminator. FIG. 15B shows the percent of GFP positive cells at different H₂O₂ concentrations as measured by flow cytometry. Different combinations of oxySp and oxySp* promoters and RBSs exhibit different H₂O₂ thresholds and basal levels for GFP activation. The oxySp* and RBS30 combination (diamonds) had the lowest threshold and a narrow transition band (shaded region).

### DETAILED DESCRIPTION

For analog gene circuits, it can be advantageous for the input to be processed over a dynamic range so that there is a significant range of input concentrations upon which to implement logic (e.g., analog-to-digital logic). The present disclosure provides gene circuits and methods for implementing wide-dynamic range behavior of gene circuits and to tune analog function. The data provided herein shows that cells can be engineered to implement synthetic computations that convert continuous information into discrete information. These computations rely, in some embodiments, on gene circuits that threshold and discretize signals from sensors, analogous to comparators in electronics. The gene circuits of the present disclosure (also referred to, in some embodiments, as “comparators”) may be adapted to other cellular contexts and for sensing inputs besides chemical concentration, such as light or contact. There are other ways to implement thresholding circuits and to dynamically alter thresholds, thus it is possible to implement a negative input terminal analogous to that in electronic comparators, rather than a fixed threshold, as provided herein.

Comparators (biological analog signal processing circuits) can be combined together to build multi-threshold analog-to-digital converters, for example. In contrast to existing technologies, the bandpass filters described below convert continuous information into distinct gene expression states instead of altering continuous gene expression. Furthermore, the outputs from the analog-to-digital converters described below can be integrated with other digital circuits. Alternatively, multiple analog signals can be integrated at the front end to calculate complex analog functions before feeding the output(s) into downstream analog-to-digital converters. The outputs of the circuits of the present disclosure are engineered, in some embodiments, to be Boolean, ternary, or multi-state digital. ADC resolution may be further increased, for example, by increasing the number of comparators across the same range of H₂O₂ or by adding comparators that can respond to lower or higher concentrations of H₂O₂.

Analog signal processing circuits of the present disclosure comprise promoters responsive to an input signal and operably linked to a nucleic acid encoding an output molecule. A “promoter” is a control region of a nucleic acid at which initiation and rate of transcription of the remainder of a nucleic acid are controlled. A promoter may also contain sub-regions at which regulatory proteins and molecules, such as transcription factors, bind. Promoters of the present disclosure may be constitutive, inducible, activatable, repressible, tissue-specific or any combination thereof. A promoter drives expression or drives transcription of the nucleic acid that it regulates. A promoter is considered to be “operably linked” when it is in a correct functional location and orientation in relation to the nucleic acid it regulates to control (“drive”) transcriptional initiation and/or expression of that nucleic acid.

A promoter is considered “responsive” to an input signal if the input signal modulates the function of the promoter, indirectly or directly. In some embodiments, an input signal may positively modulate a promoter such that the promoter activates, or increases (e.g., by a certain percentage or degree), transcription of a nucleic acid to which it is operably linked. In some embodiments, by contrast, an input signal may negatively modulate a promoter such that the promoter is prevented from activating or inhibits, or decreases, transcription of a nucleic acid to which it is operably linked. An input signal may modulate the function of the promoter directly by binding to the promoter or by acting on the promoter without an intermediate signal. For example, the oxyR protein modulates the oxyR promoter by binding to a region of the oxyR promoter. Thus, the oxyR protein is herein considered an input signal that directly modulates the oxyR promoter. By contrast, an input signal is considered to modulate the function of a promoter indirectly if the input signal modulates the promoter via an intermediate signal. For example, hydrogen peroxide (H₂O₂) modulates (e.g., activates) the oxyR protein, which, in turn, modulates (e.g., activates) the oxyR promoter. Thus, H₂O₂ is herein considered an input signal that indirectly modulates the oxyR promoter.

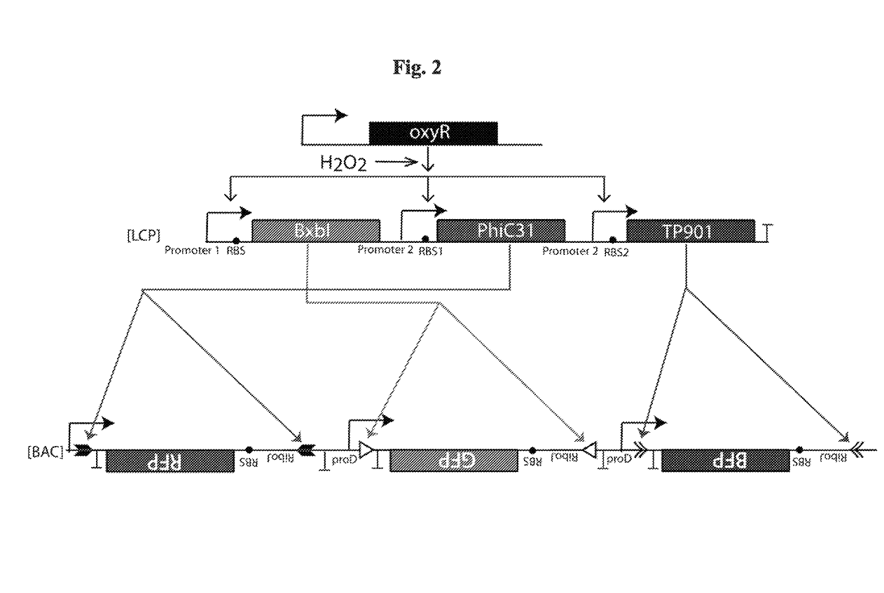

An “input signal” refers to any chemical (e.g., small molecule) or non-chemical (e.g., light or heat) signal in a cell, or to which the cell is exposed, that modulates, directly or indirectly, a component (e.g., a promoter) of an analog signal processing circuit. In some embodiments, an input signal is a biomolecule that modulates the function of a promoter (referred to as direct modulation), or is a signal that modulates a biomolecule, which then modulates the function of the promoter (referred to as indirect modulation). A “biomolecule” is any molecule that is produced in a live cell, e.g., endogenously or via recombinant-based expression. For example, with reference to FIG. 2, H₂O₂ indirectly activates transcription of an output molecule (for example RFP, GFP and/or BFP) via its activation of oxyR and subsequent binding of oxyR to the oxyR promoter or promoters. Thus, H₂O₂ is considered an input signal that indirectly modulates the oxyR promoter and, in turn, expression of output molecules. Likewise, the oxyR protein is itself considered an input signal because it directly modulates transcription of output molecules by binding to oxyR promoter(s). In some embodiments, an input signal may be endogenous to a cell or a normally exogenous condition, compound or protein that contacts a promoter of an analog signal processing circuit in such a way as to be active in modulating (e.g., inducing or repressing) transcriptional activity from a promoter responsive to the input signal (e.g., an inducible promoter). In some embodiments, an input signal is constitutively expressed in a cell. In some embodiments, the input signal is oxyR protein.

Examples of chemical input signals include, without limitation, signals extrinsic or intrinsic to a cell, such as amino acids and amino acid analogs, saccharides and polysaccharides, nucleic acids, protein transcriptional activators and repressors, cytokines, toxins, petroleum-based compounds, metal containing compounds, salts, ions, enzymes, enzyme substrates, enzyme substrate analogs, hormones, quorum-sensing molecules, proteins and small molecule drugs.

Examples of non-chemical input signals include, without limitation, changes in physiological conditions, such as changes in pH, light, temperature, radiation, osmotic pressure and saline gradients.

Promoters of the present disclosure that are responsive to an input signal and/or regulatory protein may be considered “inducible” promoters. Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some embodiments, the disclosure relates to a promoter that is operably linked to a nucleic acid encoding an output molecule (e.g., GFP or a recombinase). In some embodiments, output promoters are responsive to a regulatory protein, such as, for example, a transcription factor. In some embodiments, output promoters are modified (e.g., mutated) such that the affinity of the promoter for a particular regulatory protein is altered (e.g., reduced), relative to the affinity of the unmodified promoter for that same regulatory protein.

Promoters may contain a (e.g., at least one) modification, relative to a wild-type (unmodified) version of the same promoter (e.g., plux3 v. pluxWT). In some embodiments, the modification alters the affinity of a regulatory protein (e.g., transcription factor) for one promoter relative to another promoter (e.g., output promoter) in an analog signal processing circuit.

Promoter modifications may include, for example, single or multiple nucleotide mutations (e.g., A to T, A to C, A to G, T to A, T to C, T to G, C to A, C to T, C to G, G to A, G to T, or G to C), insertions and/or deletions (relative to an unmodified promoter) in a region, or a putative region, that affects regulatory protein binding to the region. In some embodiments, a modification is in a regulatory protein (e.g., transcription factor) binding site of a promoter. A promoter may contain a single modification or more than one (e.g., 2, 3, 4, 5, 6, 7, 8, 9, 10 or more) modifications to, for example, achieve the desired binding affinity for a cognate regulatory protein.

Modified promoters having “reduced affinity” for a cognate regulatory protein may bind to the cognate regulatory protein with an affinity that is reduced by at least 5% relative to the binding affinity of the unmodified promoter to the same cognate regulatory protein. In some embodiments, a modified promoter is considered to have reduced affinity for a cognate regulatory protein if the modified promoter binds to the cognate regulatory protein with an affinity that is reduced by at least 10% to 90%, or more (e.g., at least 10%, at least 20%, at least 30%, at least 40%, at least 50%, at least 60%, at least 70%, at least 80%, at least 90%, or more) relative to the binding affinity of the unmodified promoter to the same cognate regulatory protein.

In some aspects, a regulatory protein may have affinity for more than one naturally occurring promoter. In some embodiments of the biological circuits described herein, different naturally occurring promoters are bound by the same regulatory protein (e.g. oxySp and katGp are both bound by oxyR). In some embodiments, the different promoters are bound with different affinities by the regulatory protein. In some embodiments, the regulatory protein is oxyR. In some embodiments, the promoters are oxyR promoters. In some embodiments, the oxyR promoters are selected from the group consisting of oxySp, katGp, ahpSp, HemHp, ahpCp2, dsbGp, uofp, dpsp, grxAp, ybjCp, hcpp, ychFp, sufAp, flup, mntHp, trxCp, gorp, yhjAp, oxyRp, gntPp, uxuAp, fhuFp.

In some embodiments, the biological signal processing circuit comprises different regulatory proteins (e.g., oxyR and luxR) that sense the same input signal (e.g. H₂O₂) and bind different promoters (e.g., oxySp and plux) which have different affinities to their respective regulatory proteins.

Analog signal processing circuits, in some embodiments, are designed to detect and to generate a response to one or more input signals. For example, an analog signal processing circuit may detect and generate a response to 2, 3, 4, 5, 6, 7, 8, 9 or 10 input signals. Similarly, the present disclosure provides analog signal processing circuits having multiple output molecules (e.g., 2 to 10 output molecules).

Analog signal processing circuits of the present disclosure, in some embodiments, generate a response in the form of an output molecule. An “output molecule” refers to any detectable molecule under the control of (e.g., produced in response to) an input signal. For example, as shown in FIG. 2, RFP, GFP and BFP are output molecules produced in response to activation of the oxyR promoter by H₂O₂/oxyR protein. The expression level of an output molecule, in some embodiments, depends on the affinity of a promoter for a particular regulatory protein. For example, the expression level of an output protein under the control of a modified promoter having reduced affinity for a regulatory protein may be less than the expression level of an output molecule under the control of the unmodified promoter. Likewise, the expression level of an output molecule under the control of a modified promoter having reduced affinity for a regulatory protein may be less than the expression level of an output molecule under the control of a modified promoter having an even greater reduction in its affinity for the same regulatory protein.

Examples of output molecules include, without limitation, proteins and nucleic acids.

Examples of output protein molecules include, without limitation, marker proteins such as fluorescent proteins (e.g., GFP, EGFP, sfGFP, TagGFP, Turbo GFP, AcGFP, ZsGFP, Emerald, Azami green, mWasabi, T-Sapphire, EBFP, EBFP2, Azurite, mTagBFP, ECFP, mECFP, Cerulean, mTurquoise, CyPet, AmCyan1, Midori-ishi Cyan, TagCFP, mTFP1, EYFP, Topaz, Venus, mCitrine, YPET, TagYFP, PhiYFP, ZsYellow1, mBanana, Kusabira Orange, Orange2, mOrange, mOrange2, dTomato, dTomato-Tandem, TagRFP, TagRFP-T, DsRed, DsRed2, DsRed-Express (T1), DsRed-Monomer, mTangerine, mRuby, mApple, mStrawberry, AsRed2, mRFP1, JRed, mCherry, HcRedl, mRaspberry, dKeima-Tandem, HcRed-Tandem, mPlum, AQ143 and variants thereof), enzymes (e.g., catalytic enzymes such as recombinases, integrases, caspases), biosynthetic enzymes, cytokines, antibodies, cell receptors, regulatory proteins such as transcription factors, polymerases and chromatin remodeling factors, siRNA, trans-activating RNAs, Cas9, dCas9, guide RNAs, retrons, transposase, and microRNAs.

Recombinases are enzymes that mediate site-specific recombination by binding to nucleic acids via conserved recognition sites and mediating at least one of the following forms of DNA rearrangement: integration, excision/resolution and/or inversion. Recombinases are generally classified into two families of proteins, tyrosine recombinases (YR) and serine recombinases (SR). However, recombinases may also be classified according to their directionality (bidirectional or unidirectional).

Unidirectional recombinases bind to non-identical recognition sites and therefore mediate irreversible recombination. Examples of unidirectional recombinase recognition sites include attB, attP, attL, attR, pseudo attB, and pseudo attP. In some embodiments, the circuits described herein comprise unidirectional recombinases. Examples of unidirectional recombinases include but are not limited to BxbI, PhiC31, TP901, HK022, HP1, R4, Int1, Int2, Int3, Int4, Int5, Int6, Int7, Int8, Int9, Int10, Int11, Int12, Int13, Int14, Int15, Int16, Int17, Int18, Int19, Int20, Int21, Int22, Int23, Int24, Int25, Int26, Int27, Int28, Int29, Int30, Int31, Int32, Int33, and Int34. Further unidirectional recombinases may be identified using the methods disclosed in Yang et al., Nature Methods, October 2014; 11(12), pp. 1261-1266, herein incorporated by reference in its entirety.

In some embodiments of the circuits described herein, the circuit(s) comprise at least one unidirectional recombinase, wherein the recognition sites flanking a nucleic acid sequence are operable with the at least one unidirectional recombinase. In some embodiments, the circuit(s) comprise two or more unidirectional recombinases.

Also contemplated herein are biological signal processing circuits that are reversible. Reversible biological signal processing circuits allow the expression of an output molecule to be turned on and off, for example via the use of a “reset switch” or a second circuit that reverses the activity of an activated regulatory protein. In some embodiments, the biological signal processing circuit comprises at least one bidirectional recombinase. Bidirectional recombinases bind to identical recognition sites and therefore mediate reversible recombination. Examples of bidirectional recombinases include, but are not limited to, Cre, FLP, R, IntA, Tn3 resolvase, Hin invertase and Gin invertase. In some embodiments, the output molecule is flanked by at least one bidirectional recombinase recognition site. In some embodiments, the bidirectional recombinase recognition sites flanking an output molecule are the same. In some embodiments, the bidirectional recombinase recognition sites flanking an output molecule are different. Non-limiting examples of identical recognition sites for bidirectional recombinases include loxP, FRT and RS recognition sites. Non-limiting examples of identical recognition sites for bidirectional recombinases include loxP, FRT and RS recognition sites. It should also be noted that bidirectional recombinases can be engineered or modified to behave as unidirectional recombinases. For example, tyrosine recombinases, such as CRE can be utilized in combination with two different recombinase recognition sites (e.g. lox66 and lox71).

In some embodiments, a reversible biological analog signal processing circuit comprises a reset switch. In some embodiments, the reset switch comprises at least one recombinase directionality factor (RDF) that alters the action of a recombinase. Recombinase directionality factors are known in the art and are described, for example in Bonnet et al. PNAS 109(23), pp. 8884-9, 2012 (herein incorporated by reference in its entirety).

In some embodiments, the biological analog signal processing circuits described herein comprise bacterial recombinases. A non-limiting examples of bacterial recombinases include the FimE, FimB, FimA and HbiF. HbiF is a recombinase that reverses recombination sites that have been inverted by Fim recombinases. Bacterial recombinases recognize inverted repeat sequences, termed inverted repeat right (IRR) and inverted repeat left (IRL). In some embodiments, biological analog signal processing circuits comprising bacterial recombinases further comprise a bacterial recombinase regulator. A non-limiting example of a bacterial recombinase regulator is PapB, which inhibits FimB activity.

Examples of output nucleic acid molecules include, without limitation, RNA interference molecules (e.g., siRNA, miRNA, shRNA), guide RNA (e.g., single-stranded guide RNA), trans-activating RNAs, riboswitches, ribozymes and RNA splicing factors.

Analog signal processing circuits may contain one or multiple (e.g., 2, 3, 4 or more) copies of an output molecule. In some embodiments, analog signal processing circuits contain two or more copies of the same output molecule. In some embodiments, analog signal processing circuits contain two or more (e.g., 2, 3, 4 or more) different output molecules (e.g., 2 or more different fluorescent proteins such as GFP and mCherry, or two or more different types of output molecules such as a transcription factor or small RNAs that control transcription and a fluorescent protein). In some embodiments, an output molecule regulates expression of another output molecule (e.g., is a transcription factor that regulates a promoter, which drives expression of another output molecule). For example, in FIG. 2, BxbI, PhiC31 and TP901 are examples of output molecules that regulate expression of RFP, GFP and BFP, respectively.

Analog signal processing circuits, and components thereof, of the present disclosure can be “tuned” by promoter modification such that the affinity of a promoter for a regulatory protein differs relative to the affinity of another promoter for the same regulatory protein. Further tuning of analog signal processing circuits is contemplated herein. For example, a “regulatory sequence” may be included in a circuit to further regulate transcription, translation or degradation of an output molecule or regulatory protein. Examples of regulatory sequences as provided herein include, without limitation, ribosomal binding sites, riboswitches, ribozymes, guide RNA binding sites, microRNA binding sites, toe-hold switches, cis-repressing RNAs, siRNA binding sites, protease target sites, recombinase recognition sites and transcriptional terminator sites.

In some aspects, the disclosure relates to a biological analog signal processing circuit comprising regulatory sequences. In some embodiments, the regulatory sequences are recombinase recognition sites. In some embodiments, the recombination recognition sites recognize a recombinase selected from the group consisting of BxbI, PhiC31, TP901, BxbI, PhiC31, TP901, HK022, HP1, R4, Int1, Int2, Int3, Int4, Int5, Int6, Int7, Int8, Int9, Int10, Int11, Int12, Int13, Int14, Int15, Int16, Int17, Int18, Int19, Int20, Int21, Int22, Int23, Int24, Int25, Int26, Int27, Int28, Int29, Int30, Int31, Int32, Int33, and Int34. In some embodiments, the biological analog signal processing circuit comprises two or more different regulatory sequences. In some embodiments, the regulatory sequences regulate the transcription and/or translation of an output molecule. In some embodiments, the regulatory sequences regulate the operable linkage of a promoter to a nucleic acid sequence encoding an output protein. In some embodiments, a first set of regulatory sequences regulates the transcription and/or translation of an output molecule and a second set of regulatory sequences regulates the operable linkage of a promoter to a nucleic acid sequence encoding an output protein.

Tuning may also be achieved by modifying (e.g., mutating) a ribosomal binding site (RBS) located between a promoter and a nucleic acid to which it is operably linked. In some embodiments, the biological circuits described herein comprise RBS that have different translation efficiencies. In some embodiments, the RBS are naturally occurring RBS. In some embodiments, the RBS are modified RBS. In some embodiments, modified RBS have different translation efficiencies as a result of at least one modification relative to a wild-type (unmodified) version of the same RBS.

Tuning also can be achieved by changing the affinity of RNA polymerase for the promoter, and thus the strength of the promoter. For example, one or more mutations are made in the −10 region of the promoter. By changing the promoter strength (and thus transcription rate of the recombinase), digital switches are obtained (with regards to an input, such as H₂O₂) at different concentrations.

Tuning of an analog signal processing circuit may also be achieved, for example, by controlling the level of nucleic acid expression of particular components of the circuit. This control can be achieved, for example, by controlling copy number of the nucleic acids (e.g., using low, medium and/or high copy plasmids, and/or constitutively-active promoters).

It should be understood that the “tunability” of analog signal processing circuits of the present disclosure is achieved, in some embodiments, by combining two or more tuning mechanisms as provided herein. For example, in some embodiments, analog signal processing circuits comprise at least one modified promoter (with reduced or increased affinity for a regulatory protein) and a ribosome binding site (RBS). In some embodiments, analog signal processing circuits comprise a modified promoter and at least one modified ribosomal binding site. In some embodiments, analog signal processing circuits comprise a modified ribosomal binding site and regulatory sequence. Other configurations are contemplated herein.

Promoters of biological analog signal processing circuits may be on a vector. In some embodiments, the promoters are on the same vector (e.g., plasmid). In some embodiments, the promoters are on different vectors (e.g., each on a separate plasmid). In some embodiments, promoters may be on the same vector high copy plasmid, medium copy plasmid, or low copy plasmid. In some embodiments, output molecule(s) of biological analog signal processing circuits may be on a bacterial artificial chromosome (BAC). In some embodiments, output molecules of biological analog signal processing circuits are integrated into the genome of an organism.

For clarity and ease of explanation, promoters responsive to a regulatory protein (or responsive to an input signal) may be referred to as first, second or third promoters (and so on) so as to distinguish one promoter from another. It should be understood that reference to a first promoter and a second promoter may encompass two different promoters (e.g., oxyR v. proD). However, in some embodiments, the first promoter and second promoter are the same but can be rendered differentially responsive by other regulatory element(s) (e.g. ribosome binding sites) used in combination with the promoters. Similarly, output molecules may be referred to as a first, second or third output molecules (and so on) so as to distinguish one output molecule from another. In some embodiments, reference to a first output molecule and a second output molecule, encompasses two different output molecules (e.g., GFP v. mCherry). In some embodiments, the first and second output molecules may be the same, for example, in order to provide an extra copy of an output protein for the purpose of increased expression of said output protein.

Analog signal processing circuits of the present disclosure may be used to detect more than one input signal in a cell. For example, analog signal processing circuits may comprise one component configured to detect one input signal and another component configured to detect another input signal, each component containing a promoter (e.g., oxyR v. pLux) responsive to different regulatory proteins/input signals (e.g., oxyR/H₂O₂ v. LuxR/AHL) and operably linked to different output molecules (e.g., GFP v. mCherry). In this way, an independent response to each signal may be generated.

Thus, in some embodiments, a biological analog signal processing circuit comprising (a) a first promoter operably linked to a nucleic acid encoding a regulatory protein responsive to an input signal; (b) a second promoter responsive to the regulatory protein and operably linked to a nucleic acid encoding a first output protein, wherein activity of the second promoter is altered when bound by the regulatory protein; and, (c) an output molecule flanked by a first set of regulatory sequences, wherein the first set of regulatory sequences interacts with the first output protein to operably link the output molecule to a third promoter.

In some aspects the disclosure provides a biological analog signal processing circuit comprising (a) a first promoter operably linked to a nucleic acid encoding a regulatory protein responsive to an input signal; (b) a second promoter responsive to the regulatory protein and operably linked to a nucleic acid encoding a first output protein, wherein activity of the second promoter is altered when bound by the regulatory protein; and (c) an output molecule operably linked to a third promoter, wherein the output molecule or the third promoter is flanked by a first set of regulatory sequences, wherein the first set of regulatory sequences interacts with the first output protein to unlink the output molecule from the third promoter.

In some embodiments, the circuit further comprises: (d) a fourth promoter responsive to the regulatory protein and operably linked to a nucleic acid encoding a second output protein, wherein activity of the fourth promoter is altered when bound by the regulatory protein; and, (e) a second output molecule flanked by a second set of regulatory sequences, wherein the second set of regulatory sequences interacts with the second output protein to operably link the second output molecule to a fifth promoter.

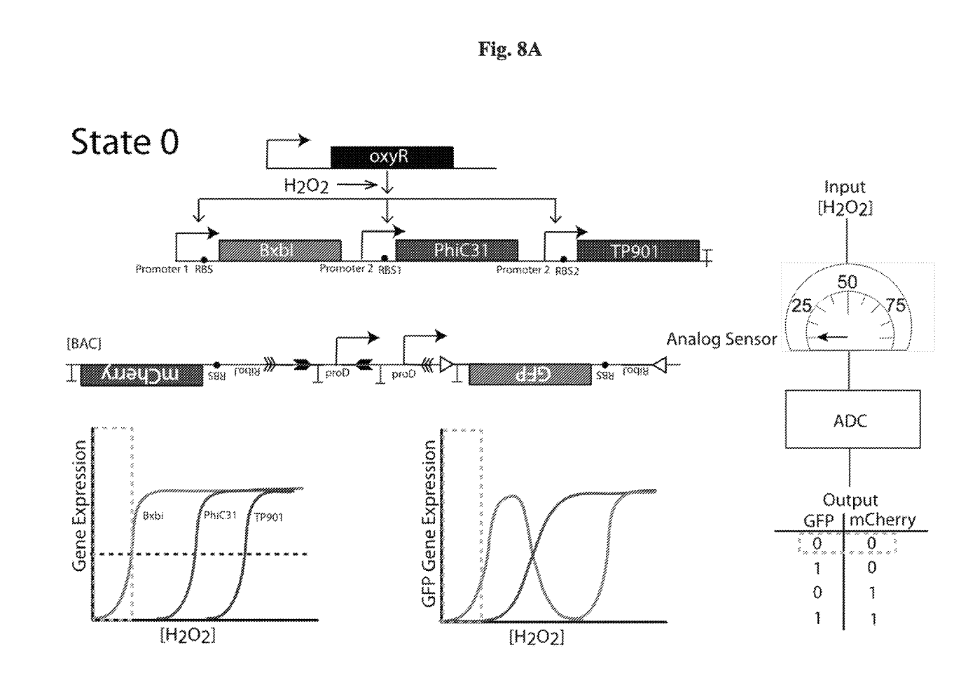

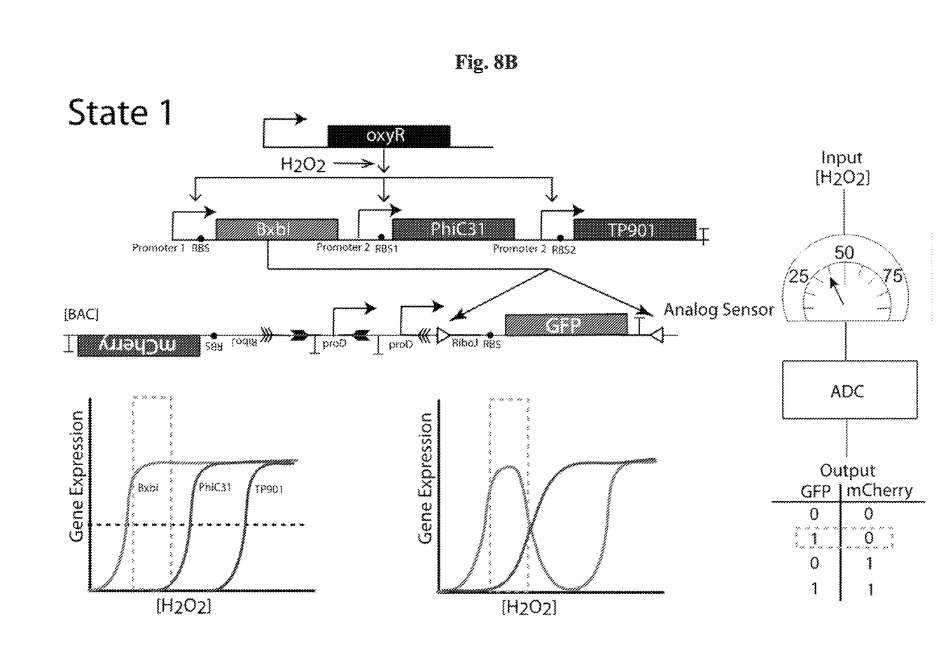

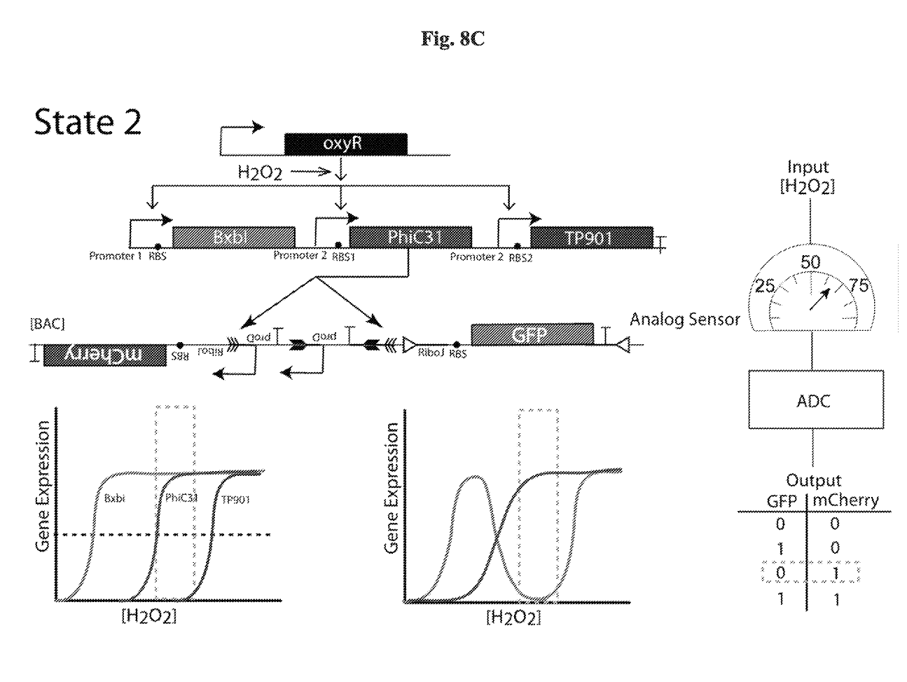

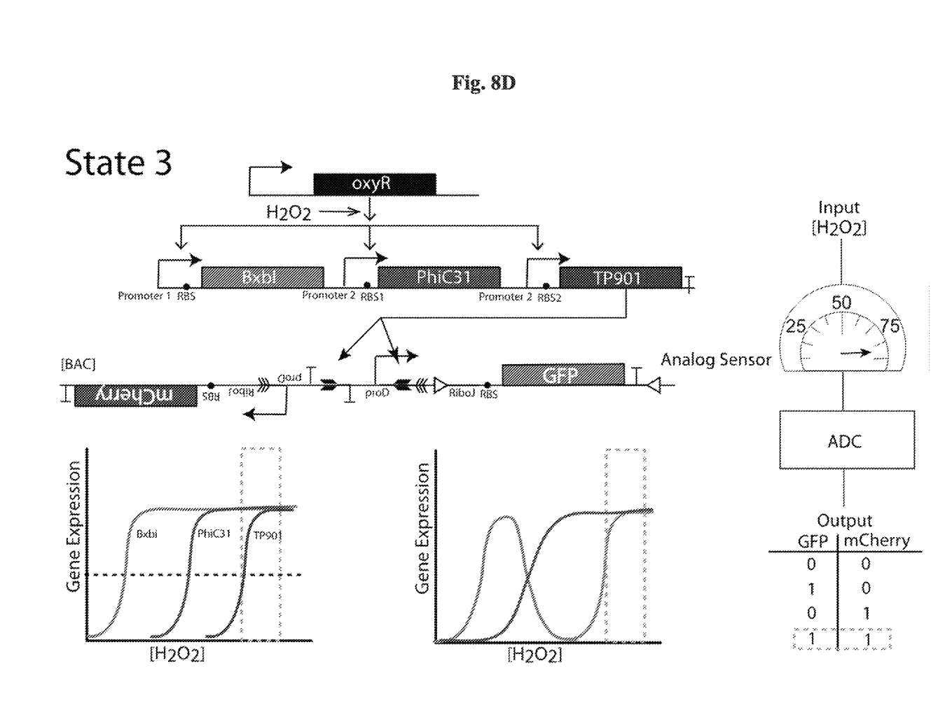

In some aspects, the tunability of the biological analog signal processing circuits described herein makes them useful for the design of a biological bandpass filter. As used herein, “bandpass filter” refers to an architecture that allows input between certain defined parameters to pass and rejects (attenuates) input outside the defined parameters. For example, FIG. 8 depicts one example of a biological bandpass filter that activates the expression of an output molecule (GFP) within a defined range of input signal concentration (H₂O₂). In some embodiments, an input signal activates a promoter operably linked to a bandpass protein, which in turn regulates the expression of an output molecule. As used herein, “bandpass protein” refers to any protein regulates expression of an output molecule within a biological bandpass filter. In some embodiments, the bandpass protein is an enzyme. In some embodiments, the bandpass protein is a recombinase.

Therefore, in some aspects, the disclosure relates to a biological bandpass filter comprising: (a) a first promoter operably linked to a nucleic acid encoding a regulatory protein responsive to an input signal; (b) a second promoter responsive to the regulatory protein and operably linked to a nucleic acid encoding a first bandpass protein, wherein activity of the second promoter is altered when bound by the regulatory protein; (c) a third promoter responsive to the regulatory protein and operably linked to a nucleic acid encoding a second bandpass protein, wherein the third promoter is not the second promoter and wherein activity of the third promoter is altered when bound by the regulatory protein; and (d) a first output molecule flanked by a first set of regulatory sequences, wherein the first set of regulatory sequences interacts with the first bandpass protein to operably link the first output molecule to a fourth promoter, wherein the fourth promoter is flanked by a second set of regulatory sequences, and wherein the second set of regulatory sequences interacts with the second bandpass protein to unlink the first output molecule from the fourth promoter.

In some aspects, the disclosure relates to a biological bandpass filter comprising: (a) a first promoter operably linked to a nucleic acid encoding a regulatory protein responsive to an input signal; (b) a second promoter responsive to the regulatory protein and operably linked to a nucleic acid encoding a first bandpass protein, wherein activity of the second promoter is altered when bound by the regulatory protein; (c) a third promoter responsive to the regulatory protein and operably linked to a nucleic acid encoding a second bandpass protein, wherein the third promoter is not the second promoter and wherein activity of the third promoter is altered when bound by the regulatory protein; and (d) a first output molecule operably linked to a fourth promoter, flanked by a first set of regulatory sequences, wherein the first set of regulatory sequences interacts with the first bandpass protein to unlink the first output molecule from the fourth promoter, wherein the first set of regulatory sequences is flanked by a second set of regulatory sequences, and wherein the second set of regulatory sequences interacts with the second bandpass protein to operably link the first output molecule to the fourth promoter.

Also contemplated herein is the combination of a biological analog signal processing circuit with a biological bandpass filter. Accordingly, in some aspects, the disclosure relates to a biological analog signal processing circuit comprising: (a) a first promoter operably linked to a nucleic acid encoding a regulatory protein responsive to an input signal; (b) a second promoter responsive to the regulatory protein and operably linked to a nucleic acid encoding a first bandpass protein, wherein activity of the second promoter is altered when bound by the regulatory protein; (c) a third promoter responsive to the regulatory protein and operably linked to a nucleic acid encoding a second bandpass protein, wherein the third promoter is not the second promoter and wherein activity of the third promoter is altered when bound by the regulatory protein; (d) a fourth promoter responsive to the regulatory protein and operably linked to a nucleic acid encoding a third bandpass protein, wherein the fourth promoter is not the first and not the second promoter and wherein activity of the fourth promoter is altered when bound by the regulatory protein; (e) a first output molecule flanked by a first set of regulatory sequences, wherein the first set of regulatory sequences interacts with the first bandpass protein to operably link the first output molecule to a fifth promoter, wherein the fifth promoter and a sixth promoter are flanked by a second set of regulatory sequences, wherein the second set of regulatory sequences interacts with the second bandpass protein to unlink the fifth promoter from the first output molecule and to operably link the fifth promoter to a second output molecule; and, (f) a third set of regulatory sequences flanking the sixth promoter, wherein the third set of regulatory sequences interacts with the third bandpass protein to operably link the sixth promoter to the first output molecule without unlinking the fifth promoter from the second output molecule.

In some embodiments, the disclosure contemplates the combination of more than one biological analog signal processing circuit and/or biological bandpass filter within a cell. In some embodiments, two biological analog signal processing circuits are combined within a cell. In some embodiments, two biological analog signal processing circuits are combined with at least one biological bandpass filter. In some embodiments, 1, 2, 3, 4, 5, 6, 7, 8, 9 or 10 biological analog signal processing circuits are combined alone, or in combination with 1, 2, 3, 4, 5, 6, 7, 8, 9 or 10 biological bandpass filters.

Analog signal processing circuits of the present disclosure may be expressed in a broad range of host cell types. In some embodiments, analog signal processing circuits are expressed in bacterial cells, yeast cells, insect cells, mammalian cells or other types of cells.

Bacterial cells of the present disclosure include bacterial subdivisions of Eubacteria and Archaebacteria. Eubacteria can be further subdivided into gram-positive and gram-negative Eubacteria, which depend upon a difference in cell wall structure. Also included herein are those classified based on gross morphology alone (e.g., cocci, bacilli). In some embodiments, the bacterial cells are Gram-negative cells, and in some embodiments, the bacterial cells are Gram-positive cells. Examples of bacterial cells of the present disclosure include, without limitation, cells from Yersinia spp., Escherichia spp., Klebsiella spp., Acinetobacter spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Salmonella spp., Vibrio spp., Bacillus spp., Erysipelothrix spp., Salmonella spp., Streptomyces spp., Bacteroides spp., Prevotella spp., Clostridium spp., Bifidobacterium spp., or Lactobacillus spp. In some embodiments, the bacterial cells are from Bacteroides thetaiotaomicron, Bacteroides fragilis, Bacteroides distasonis, Bacteroides vulgatus, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinobycetemcomitans, cyanobacteria, Escherichia coli, Helicobacter pylori, Selnomonas ruminatium, Shigella sonnei, Zymomonas mobilis, Mycoplasma mycoides, Treponema denticola, Bacillus thuringiensis, Staphylococcus lugdunensis, Leuconostoc oenos, Corynebacterium xerosis, Lactobacillus plantarum, Lactobacillus rhamnosus, Lactobacillus casei, Lactobacillus acidophilus, Streptococcus spp., Enterococcus faecalis, Bacillus coagulans, Bacillus ceretus, Bacillus popillae, Synechocystis strain PCC6803, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Zymomonas mobilis, Streptomyces phaechromogenes, or Streptomyces ghanaenis. “Endogenous” bacterial cells refer to non-pathogenic bacteria that are part of a normal internal ecosystem such as bacterial flora.

In some embodiments, bacterial cells of the present disclosure are anaerobic bacterial cells (e.g., cells that do not require oxygen for growth). Anaerobic bacterial cells include facultative anaerobic cells such as, for example, Escherichia coli, Shewanella oneidensis and Listeria monocytogenes. Anaerobic bacterial cells also include obligate anaerobic cells such as, for example, Bacteroides and Clostridium species. In humans, for example, anaerobic bacterial cells are most commonly found in the gastrointestinal tract.

In some embodiments, analog signal processing circuits are expressed in mammalian cells. For example, in some embodiments, analog signal processing circuits are expressed in human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, engineered constructs are expressed in human embryonic kidney (HEK) cells (e.g., HEK 293 or HEK 293T cells). In some embodiments, engineered constructs are expressed in stem cells (e.g., human stem cells) such as, for example, pluripotent stem cells (e.g., human pluripotent stem cells including human induced pluripotent stem cells (hiPSCs)). A “stem cell” refers to a cell with the ability to divide for indefinite periods in culture and to give rise to specialized cells. A “pluripotent stem cell” refers to a type of stem cell that is capable of differentiating into all tissues of an organism, but not alone capable of sustaining full organismal development. A “human induced pluripotent stem cell” refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepalc1c7, High Five, HL-60, HMEC, HT-29, HUVEC, J558L, Jurkat, JY cells, K562, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRCS, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL, RenCa, RIN-5F, RMA/RMAS, S2, Saos-2, Sf21, Sf9, SiHa, SKBR3, SKOV-3, T-47D, T2, T84, THP1, U373, U87, U937, VCaP, WM39, WT-49, X63, YAC-1 and YAR cells.

In other embodiments, the cell is a fungal cell such as a yeast cell, e.g., Saccharomyces spp., Schizosaccharomyces spp., Pichia spp., Komagataella spp., Phaffia spp., Kluyveromyces spp., Candida spp., Talaromyces spp., Brettanomyces spp., Pachysolen spp., Debaryomyces spp., Yarrowia spp., and industrial polyploid yeast strains. Other examples of fungi include Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp. Cells of the present disclosure are generally considered to be modified. A modified cell is a cell that contains an exogenous nucleic acid or a nucleic acid that does not occur in nature (e.g., an analog signal processing circuit of the present disclosure). In some embodiments, a modified cell contains a mutation in a genomic nucleic acid. In some embodiments, a modified cell contains an exogenous independently replicating nucleic acid (e.g., components of analog signal processing circuits present on an episomal vector). In some embodiments, a modified cell is produced by introducing a foreign or exogenous nucleic acid into a cell. Thus, provided herein are methods of introducing an analog signal processing circuit into a cell. A nucleic acid may be introduced into a cell by conventional methods, such as, for example, electroporation (see, e.g., Heiser W. C. Transcription Factor Protocols: Methods in Molecular Biology™ 2000; 130: 117-134), chemical (e.g., calcium phosphate or lipid) transfection (see, e.g., Lewis W. H., et al., Somatic Cell Genet. 1980 May; 6(3): 333-47; Chen C., et al., Mol Cell Biol. 1987 August; 7(8): 2745-2752), fusion with bacterial protoplasts containing recombinant plasmids (see, e.g., Schaffner W. Proc Natl Acad Sci USA. 1980 April; 77(4): 2163-7), transduction, conjugation, or microinjection of purified DNA directly into the nucleus of the cell (see, e.g., Capecchi M. R. Cell. 1980 November; 22(2 Pt 2): 479-88).

In some embodiments, a cell is modified to overexpress an endogenous protein of interest (e.g., via introducing or modifying a promoter or other regulatory element near the endogenous gene that encodes the protein of interest to increase its expression level). In some embodiments, a cell is modified by mutagenesis. In some embodiments, a cell is modified by introducing an engineered nucleic acid into the cell in order to produce a genetic change of interest (e.g., via insertion or homologous recombination).

In some embodiments, a cell contains a gene deletion.

Analog signal processing circuits of the present disclosure may be transiently expressed or stably expressed. “Transient cell expression” refers to expression by a cell of a nucleic acid that is not integrated into the nuclear genome of the cell. By comparison, “stable cell expression” refers to expression by a cell of a nucleic acid that remains in the nuclear genome of the cell and its daughter cells. Typically, to achieve stable cell expression, a cell is co-transfected with a marker gene and an exogenous nucleic acid (e.g., an analog signal processing circuit or component thereof) that is intended for stable expression in the cell. The marker gene gives the cell some selectable advantage (e.g., resistance to a toxin, antibiotic, or other factor). Few transfected cells will, by chance, have integrated the exogenous nucleic acid into their genome. If a toxin, for example, is then added to the cell culture, only those few cells with a toxin-resistant marker gene integrated into their genomes will be able to proliferate, while other cells will die. After applying this selective pressure for a period of time, only the cells with a stable transfection remain and can be cultured further. Examples of marker genes and selection agents for use in accordance with the present disclosure include, without limitation, dihydrofolate reductase with methotrexate, glutamine synthetase with methionine sulphoximine, hygromycin phosphotransferase with hygromycin, puromycin N-acetyltransferase with puromycin, and neomycin phosphotransferase with Geneticin, also known as G418. Other marker genes/selection agents are contemplated herein.

Expression of nucleic acids in transiently-transfected and/or stably-transfected cells may be constitutive or inducible. Inducible promoters for use as provided herein are described above.

In some aspects, the disclosure relates to a method of analog signal processing in cells, comprising: providing a cell or cell lysate that comprises the circuit of any one of the preceding claims; and contacting the cell with an input signal that modulates the regulatory protein.

In some embodiments, the method further comprises contacting the cell or cell lysate with different concentrations of the input signal.

In some embodiments, the method comprises detecting in the cell or cell lysate an expression level of the output molecule and, optionally, quantifying levels of the output molecule.

In some embodiments of the method, the cell is a bacterial cell. In some embodiments, the bacterial cell is an Escherichia coli cell. In some embodiments, the output molecule is a reporter molecule, an enzyme, a therapeutic molecule or a nucleic acid molecule.

In some embodiments, provided herein are methods of delivering analog signal processing circuits (e.g., containing an analog correction component) to a subject (e.g., a human subject). Analog signal processing circuits may be delivered to subjects using, for example, in bacteriophage or phagemid vehicles, or other delivery vehicle that is capable of delivering nucleic acids to a cell in vivo. In some embodiments, analog signal processing circuits may be introduced into cells ex vivo, which cells are then delivered to a subject via injection, oral delivery, or other delivery route or vehicle.

Other uses of analog signal processing circuits are contemplated by the present disclosure. For example, the present disclosure provides cells engineered to dynamically control the synthesis of molecules or peptides based on intrinsic factors (e.g., the concentration of metabolic intermediates) or extrinsic factors (e.g., inducers); analog signal processing circuits engineered to classify a cell type (e.g., via inputs from outside of the cell, such as receptors, or inputs from inside of the cell, such as transcription factors, DNA sequence and RNAs); and cells engineered to synthesize materials in a spatial pattern based on, for example, environmental cues.

It should be understood that while analog signal processing circuits of the present disclosure, in many embodiments, are delivered to cells or are otherwise used in vivo, the invention is not so limited. Analog signal processing circuits as provided herein may be used in vivo or in vitro, intracellularly or extracellularly (e.g., using cell-free extracts/lysates). For example, analog signal processing circuits may be used in an in vitro abiotic paper-based platform as described in Pardee K et al. (Cell. 2014 Nov. 6; 159(4):940-54. doi: 10.1016/j.cell.2014.10.004. Epub 2014 Oct. 23, incorporated by reference herein) to, for example, enable rapid prototyping for cell-based research and gene circuit design.

The present disclosure also provides aspects encompassed by the following numbered paragraphs:

1. A biological analog signal processing circuit comprising: (a) a first promoter operably linked to a nucleic acid encoding a regulatory protein responsive to an input signal; (b) a second promoter responsive to the regulatory protein and operably linked to a nucleic acid encoding a first output protein, wherein activity of the second promoter is altered when bound by the regulatory protein; and, (c) an output molecule flanked by a first set of regulatory sequences, wherein the first set of regulatory sequences interacts with the first output protein to operably link the output molecule to a third promoter.

2. A biological analog signal processing circuit comprising: (a) a first promoter operably linked to a nucleic acid encoding a regulatory protein responsive to an input signal; (b) a second promoter responsive to the regulatory protein and operably linked to a nucleic acid encoding a first output protein, wherein activity of the second promoter is altered when bound by the regulatory protein; and (c) an output molecule operably linked to a third promoter, wherein the output molecule or the third promoter is flanked by a first set of regulatory sequences, wherein the first set of regulatory sequences interacts with the first output protein to unlink the output molecule from the third promoter.

3. The circuit of paragraph 1 further comprising: (d) a fourth promoter responsive to the regulatory protein and operably linked to a nucleic acid encoding a second output protein, wherein activity of the fourth promoter is altered when bound by the regulatory protein; and (e) a second output molecule flanked by a second set of regulatory sequences, wherein the second set of regulatory sequences interacts with the second output protein to operably link the second output molecule to a fifth promoter.

4. The circuit of any one of paragraphs 1-3, wherein the promoter of (a) is a constitutively-active promoter.

5. The circuit of any one of paragraphs 1-4, wherein the regulatory protein is oxyR.

6. The circuit of any one of paragraphs 1-5, wherein the promoter of (b) and/or (d) comprises a modification that alters the binding affinity of a transcription factor or RNA polymerase for the promoter of (b) and/or (d), relative to a similar unmodified promoter.

7. The circuit of paragraph 6, wherein the modification is a nucleic acid mutation.

8. The circuit of any one of paragraphs 1-7, wherein (a), (b) and (c) are on a vector.

9. The circuit of paragraph 7, wherein (a), (b), (c) and (d) are on a vector.

10. The circuit of any one of paragraphs 1-9, wherein (a) and (b) are on a single vector.

11. The circuit of paragraph 10, wherein (a), (b) and (d) are on a single vector.

12. The circuit of any one of paragraphs 8-11, wherein the vector is a low copy plasmid, a medium copy plasmid or a high copy plasmid.

13. The circuit of any one of paragraphs 1-12, wherein (c) and/or (e) is on a bacterial artificial chromosome (BAC).

14. The circuit of any one of paragraphs 1-13, wherein (b) and/or (d) further comprises a sequence element that regulates production of the first output protein and is located between the second promoter and the nucleic acid encoding the first output protein.

15. The circuit of paragraph 14, wherein the sequence element regulates transcription or translation of the output protein.

16. The circuit of paragraph 14 or 15, wherein the sequence element is a ribosomal binding site.

17. The circuit of paragraph 16, wherein the sequence element is a modified ribosomal binding site comprising a modification that alters the binding affinity of a ribosome for the modified ribosomal binding site, relative to a similar unmodified ribosomal binding site.

18. The circuit of any one of paragraphs 1-17, wherein the promoter of (b) and/or (d) is an oxyR promoter that comprises a modification that alters the binding affinity of a transcription factor or RNA polymerase for oxyR promoter of (b), relative to a similar unmodified promoter.

19. The circuit of any one of paragraphs 1-18, wherein the first output protein of (b) is a recombinase and the first set of regulatory sequences of (c) is recombinase recognition sites.

20. The circuit of any one of paragraphs 1-19, wherein the second output protein of (d) is a recombinase and the second set of regulatory sequences of (e) is recombinase recognition sites.

21. The circuit of any one of paragraphs 1-20, wherein the first output molecule of (c) is a fluorescent protein.

22. The circuit of any one of paragraphs 2-21, wherein the second output molecule of (e) is a fluorescent protein.

23. A cell or cell lysate comprising the circuit of any one of paragraphs 1-22.

24. The cell or cell lysate of paragraph 23, wherein the cell is a bacterial cell.

25. The cell or cell lysate of paragraph 24, wherein the bacterial cell is an Escherichia coli cell.

26. The cell or cell lysate of any one of paragraphs 22-25 further comprising the input signal.

27. The cell or cell lysate of paragraph 26, wherein the input signal modulates activity of the regulatory protein.

28. The cell or cell lysate of paragraph 27, wherein the input signal activates activity of the regulatory protein.

29. The cell or cell lysate of paragraphs 26-28, wherein the input signal is a chemical input signal.

30. The cell or cell lysate of paragraph 29, wherein the chemical input signal is hydrogen peroxide.

31. A biological bandpass filter comprising: (a) a first promoter operably linked to a nucleic acid encoding a regulatory protein responsive to an input signal; (b) a second promoter responsive to the regulatory protein and operably linked to a nucleic acid encoding a first bandpass protein, wherein activity of the second promoter is altered when bound by the regulatory protein; (c) a third promoter responsive to the regulatory protein and operably linked to a nucleic acid encoding a second bandpass protein, wherein the third promoter is not the second promoter and wherein activity of the third promoter is altered when bound by the regulatory protein; and (d) a first output molecule flanked by a first set of regulatory sequences, wherein the first set of regulatory sequences interacts with the first bandpass protein to operably link the first output molecule to a fourth promoter, wherein the fourth promoter is flanked by a second set of regulatory sequences, and wherein the second set of regulatory sequences interacts with the second bandpass protein to unlink the first output molecule from the fourth promoter.

32. A biological bandpass filter comprising: (a) a first promoter operably linked to a nucleic acid encoding a regulatory protein responsive to an input signal; (b) a second promoter responsive to the regulatory protein and operably linked to a nucleic acid encoding a first bandpass protein, wherein activity of the second promoter is altered when bound by the regulatory protein; (c) a third promoter responsive to the regulatory protein and operably linked to a nucleic acid encoding a second bandpass protein, wherein the third promoter is not the second promoter and wherein activity of the third promoter is altered when bound by the regulatory protein; and (d) a first output molecule operably linked to a fourth promoter, flanked by a first set of regulatory sequences, wherein the first set of regulatory sequences interacts with the first bandpass protein to unlink the first output molecule from the fourth promoter, wherein the first set of regulatory sequences is flanked by a second set of regulatory sequences, and wherein the second set of regulatory sequences interacts with the second bandpass protein to operably link the first output molecule to the fourth promoter.

33. The circuit of paragraph 31 or 32, wherein the promoter of (a) is a constitutively-active promoter.

34. The circuit of any one of paragraphs 31-33, wherein the regulatory protein is oxyR.

35. The circuit of any one of paragraphs 31-34, wherein the second promoter of (b) and/or the third promoter of (c) comprises a modification that alters the binding affinity of a transcription factor or RNA polymerase for the second promoter of (b) and or the third promoter of (c) relative to a similar unmodified promoter.

36. The circuit of paragraph 35, wherein the modification is a nucleic acid mutation.

37. The circuit of any one of paragraphs 31-36, wherein (a), (b) and (c) are on a vector.

38. The circuit of any one of paragraphs 31-37, wherein (a), (b) and (c) are on the same vector.

39. The circuit of paragraph 37 or 38, wherein the vector is a low copy plasmid, a medium copy plasmid or a high copy plasmid.

40. The circuit of any one of paragraphs 31-39, wherein (d) is on a bacterial artificial chromosome (BAC).

41. The circuit of any one of paragraphs 31-40, wherein (b) further comprises a sequence element that regulates production of the first bandpass protein and is located between the second promoter and the nucleic acid encoding the first bandpass protein.

42. The circuit of any one of paragraphs 31-42, wherein (c) further comprises a sequence element that regulates production of the second bandpass protein and is located between the third promoter and the nucleic acid encoding the second bandpass protein.

43. The circuit of paragraph 41 or 42, wherein the sequence element regulates transcription or translation of the first bandpass protein and/or second bandpass protein.

44. The circuit of paragraph 43, wherein the sequence element is a ribosomal binding site.

45. The circuit of paragraph 44, wherein the sequence element is a modified ribosomal binding site comprising a modification that alters the binding affinity of a ribosome for the modified ribosomal binding site, relative to a similar unmodified ribosomal binding site.

46. The circuit of any one of paragraphs 31-45, wherein the promoter of (b) is an oxyR promoter that comprises a modification that alters the binding affinity of a transcription factor or RNA polymerase for oxyR promoter of (b), relative to a similar unmodified promoter.

47. The circuit of any one of paragraphs 31-46, wherein the promoter of (c) is an oxyR promoter that comprises a modification that alters the binding affinity of a transcription factor or RNA polymerase for oxyR promoter of (c), relative to a similar unmodified promoter.

48. The circuit of any one of paragraphs 31-47, wherein the first bandpass protein of (b) is a recombinase.

49. The circuit of any one of paragraphs 31-48, wherein the second bandpass protein of (c) is a recombinase.

50. The circuit of any one of paragraphs 31-49, wherein the first set of regulatory sequences and/or the second set of regulatory sequences of (d) are recombinase recognition sites.

51. The circuit of any one of paragraphs 31-50, wherein the first output protein of (d) is a fluorescent protein.

52. A cell or cell lysate comprising the circuit of any one of paragraphs 31-51.

53. The cell or cell lysate of paragraph 52, wherein the cell is a bacterial cell.

54. The cell or cell lysate of paragraph 53, wherein the bacterial cell is an Escherichia coli cell.

55. The cell or cell lysate of any one of paragraphs 52-54 further comprising the input signal.

56. The cell or cell lysate of paragraph 55, wherein the input signal modulates activity of the regulatory protein.

57. The cell or cell lysate of paragraph 56, wherein the input signal activates activity of the regulatory protein.

58. The cell or cell lysate of any one of paragraphs 55-57, wherein the input signal is a chemical input signal.

59. The cell or cell lysate of paragraph 58, wherein the chemical input signal is hydrogen peroxide.

60. A biological analog signal processing circuit comprising: (a) a first promoter operably linked to a nucleic acid encoding a regulatory protein responsive to an input signal; (b) a second promoter responsive to the regulatory protein and operably linked to a nucleic acid encoding a first bandpass protein, wherein activity of the second promoter is altered when bound by the regulatory protein; (c) a third promoter responsive to the regulatory protein and operably linked to a nucleic acid encoding a second bandpass protein, wherein the third promoter is not the second promoter and wherein activity of the third promoter is altered when bound by the regulatory protein; (d) a fourth promoter responsive to the regulatory protein and operably linked to a nucleic acid encoding a third bandpass protein, wherein the fourth promoter is not the first and not the second promoter and wherein activity of the fourth promoter is altered when bound by the regulatory protein; (e) a first output molecule flanked by a first set of regulatory sequences, wherein the first set of regulatory sequences interacts with the first bandpass protein to operably link the first output molecule to a fifth promoter, wherein the fifth promoter and a sixth promoter are flanked by a second set of regulatory sequences, wherein the second set of regulatory sequences interacts with the second bandpass protein to unlink the fifth promoter from the first output molecule and to operably link the fifth promoter to a second output molecule; and, (f) a third set of regulatory sequences flanking the sixth promoter, wherein the third set of regulatory sequences interacts with the third bandpass protein to operably link the sixth promoter to the first output molecule without unlinking the fifth promoter from the second output molecule.

61. The circuit of paragraph 60, wherein the promoter of (a) is a constitutively-active promoter.

62. The circuit of paragraph 60 or 61, wherein the regulatory protein is oxyR.

63. The circuit of any one of paragraphs 60-62, wherein the second promoter of (b) and/or the third promoter of (c) and/or the fourth promoter of (d) comprises a modification that alters the binding affinity of a transcription factor or RNA polymerase for the promoter of (b) and/or (c) and/or (d), relative to a similar unmodified promoter.

64. The circuit of paragraph 63, wherein the modification is a nucleic acid mutation.

65. The circuit of any one of paragraphs 60-64, wherein (a), (b), (c) and/or (d) are on a vector.

66. The circuit of any one of paragraphs 60-65, wherein (a), (b), (c) and/or (d) are on the same vector.

67. The circuit of paragraph 65 or 66, wherein the vector is a low copy plasmid, a medium copy plasmid or a high copy plasmid.

68. The circuit of any one of paragraphs 60-67, wherein (e) and (f) are on a bacterial artificial chromosome (BAC).

69. The circuit of paragraph 68, wherein (e) and (f) are on a single bacterial artificial chromosome (BAC).

70. The circuit of any one of paragraphs 60-69, wherein (b) further comprises a sequence element that regulates production of the first bandpass protein and is located between the second promoter and the nucleic acid encoding the first bandpass protein.

71. The circuit of any one of paragraphs 60-70, wherein (c) further comprises a sequence element that regulates production of the second bandpass protein and is located between the third promoter and the nucleic acid encoding the second bandpass protein.

72. The circuit of any one of paragraphs 60-71, wherein (d) further comprises a sequence element that regulates production of the third bandpass protein and is located between the fourth promoter and the nucleic acid encoding the third bandpass protein.

73. The circuit of any one of paragraphs 60-72, wherein the sequence element regulates transcription or translation of the first bandpass protein and/or the second bandpass protein and/or the third bandpass protein.

74. The circuit of any one of paragraphs 60-73, wherein the sequence element is a ribosomal binding site.

75. The circuit of paragraph 74, wherein the sequence element is a modified ribosomal binding site comprising a modification that alters the binding affinity of a ribosome for the modified ribosomal binding site, relative to a similar unmodified ribosomal binding site.

76. The circuit of any one of paragraphs 60-75, wherein the promoter of (b) is an oxyR promoter that comprises a modification that alters the binding affinity of a transcription factor or RNA polymerase for oxyR promoter of (b), relative to a similar unmodified promoter.

77. The circuit of any one of paragraphs 60-76, wherein the promoter of (c) is an oxyR promoter that comprises a modification that alters the binding affinity of a transcription factor for or RNA polymerase oxyR promoter of (c), relative to a similar unmodified promoter.

78. The circuit of any one of paragraphs 60-77, wherein the promoter of (d) is an oxyR promoter that comprises a modification that alters the binding affinity of a transcription factor for or RNA polymerase oxyR promoter of (d), relative to a similar unmodified promoter.

79. The circuit of any one of paragraphs 60-78, wherein the first bandpass protein of (b) is a recombinase.

80. The circuit of any one of paragraphs 60-79, wherein the second bandpass protein of (c) is a recombinase.

81. The circuit of any one of paragraphs 60-80, wherein the third bandpass protein of (d) is a recombinase.

82. The circuit of any one of paragraphs 60-81, wherein the first set of regulatory sequences and/or the second set of regulatory sequences of (d) and/or the third set of regulatory sequences of (e) are recombinase recognition sites.

83. The circuit of any one of paragraphs 60-82, wherein the first output molecule of (e) is a fluorescent protein.

84. A cell or cell lysate comprising the circuit of any one of paragraphs 60-83.

85. The cell or cell lysate of paragraph 84, wherein the cell is a bacterial cell.

86. The cell or cell lysate of paragraph 85, wherein the bacterial cell is an Escherichia coli cell.

87. The cell or cell lysate of any one of paragraphs 84 to 86 further comprising the input signal.

88. The cell or cell lysate of paragraph 87, wherein the input signal modulates activity of the regulatory protein.

89. The cell or cell lysate of paragraph 88, wherein the input signal activates activity of the regulatory protein.

90. The cell or cell lysate of any one of paragraphs 87-89, wherein the input signal is a chemical input signal.

91. The cell or cell lysate of paragraph 90, wherein the chemical input signal is hydrogen peroxide.

92. A method of analog signal processing in cells, comprising: providing a cell or cell lysate that comprises the circuit of any one of the preceding paragraphs; and contacting the cell with an input signal that modulates the regulatory protein.

93. The method of paragraph 92 further comprising contacting the cell or cell lysate with different concentrations of the input signal.

94. The method of paragraph 92 or 93 further comprising detecting in the cell or cell lysate an expression level of the output molecule and, optionally, quantifying levels of the output molecule.

95. The method of any one of paragraphs 92-94, wherein the cell is a bacterial cell.

96. The method of paragraph 95, wherein the bacterial cell is an Escherichia coli cell.

97. The method of any one of paragraphs 92-96, wherein the output molecule is a reporter molecule, an enzyme, a therapeutic molecule or a nucleic acid molecule.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teachings that are referenced herein.

### EXAMPLES

Analog and digital computation each have distinct advantages for cellular computing. Digital computation in natural biological systems is useful for signal integration given its relative robustness to noise and is exemplified by decision-making circuits, such as those in developmental programs that lead cells into differentiated states. Analog computation is useful for signal processing in natural biological systems when the output needs to be dependent on graded information or continuous functions of the inputs, such as the sum or ratio of energy sources. However, analog signal integration is susceptible to noise, making it challenging to design robust synthetic genetic programs. Here, we combine the benefits of analog signal processing with digital signal integration to create artificial mixed-signal gene networks that carry out new hybrid functions in living cells.

Signals are processed from front-end analog sensors with composable input-discretization devices that are analogous to electronic comparators. The outputs of these devices can then be processed in a digital fashion with downstream circuits. This strategy of explicitly digitizing analog signals followed by digital computing stages is conceptually different than other mixed-signal computing approaches, such as fuzzy logic, neural networks, and hybrid automata, in which analog and digital processing are intricately coupled. The components developed herein may be useful for future gene circuits implementing the latter form of hybrid computing. Electronic comparators compare analog voltages between two terminals (V₊ and V₋) and output a digital OFF or ON signal (or “LO” or “HI”) if V₊<V₋ or V₊>V₋, respectively. Rather than voltage, genetic comparators take the concentration of an activated transcription factor as their input, for example. The transcription factor acts a front-end sensor for continuous information (e.g., the concentration of a small molecule), and operates over a wide input dynamic range to enable multiple genetic comparators with different thresholds to discretize the same input into multiple distinct outputs. Comparators convert molecular concentration into digital gene expression. This enables the creation higher-order mixed-signal circuits that also take on digital gene expression states.

### Example 1: Framework for Engineering Complex, Robust Cellular Computation

Biology uses a mixed signal approach to understand an environment and implement an appropriate response. This mixed signal approach is a combination of analog and digital signal processing.

Most work in gene circuit design is focused on digital signal processing. In the present disclosure, methods to integrate front-end analog processing with digital signal processing in living cells are described.

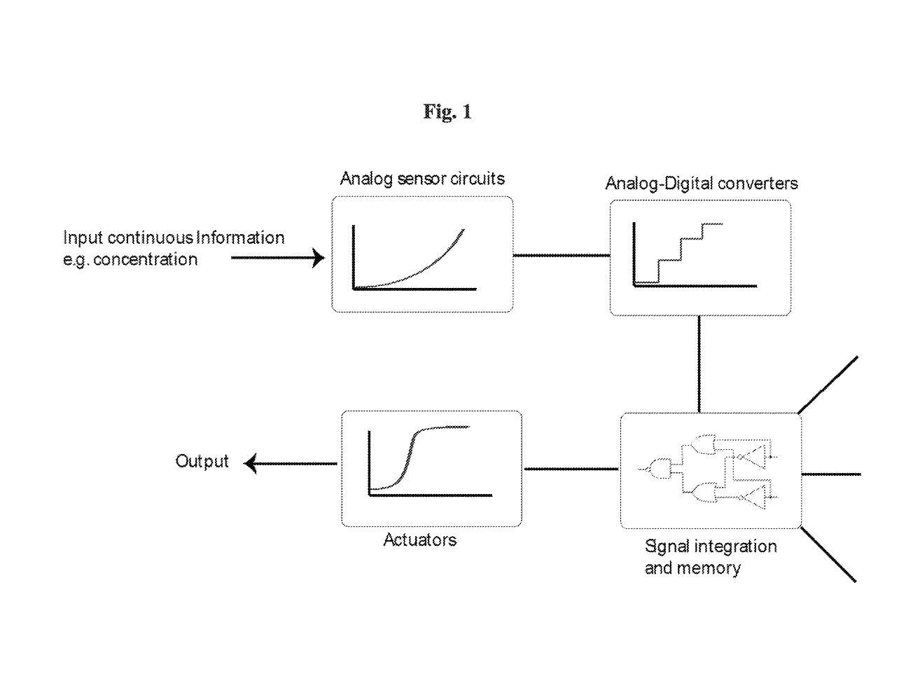

FIG. 1 shows an example framework of a biological analog signal processing circuit, with cells processing continuous input molecular concentrations with analog sensors. The analog sensors are processed by analog-to-digital converters to remove noise. The output of analog-to-digital converts are integrated with other sensors, and cells make a decision based on this information. This decision leads to the production of an “output” for actuation.

The advantages of the mixed signal approach are numerous and include noise mitigation, decision making and linear classification. The ability to set and tune thresholds is key to mixed signal processing.

Fundamental methods to integrate analog and digital processing and to scale these circuits to build higher order gene networks are described below.

### Example 2: A Biological Circuit for Converting Analog Input Concentration to Discrete Digital Gene Expression Regimes

FIG. 2 provides a schematic representation of a biological circuit for converting analog input concentration to digital gene expression. The oxyR transcription factor is constitutively produced. It senses H₂O₂ and actives several promoters (for example, oxySp, katGp, and ahpCp) with different affinities. The affinity with which oxyR binds to a particular promoter determines the expression level of the molecule operably linked to that promoter. Expression levels of output molecules can be further modified by the addition of regulatory sequences, such as ribosome binding sites and recombinase recognition sites to the circuit.

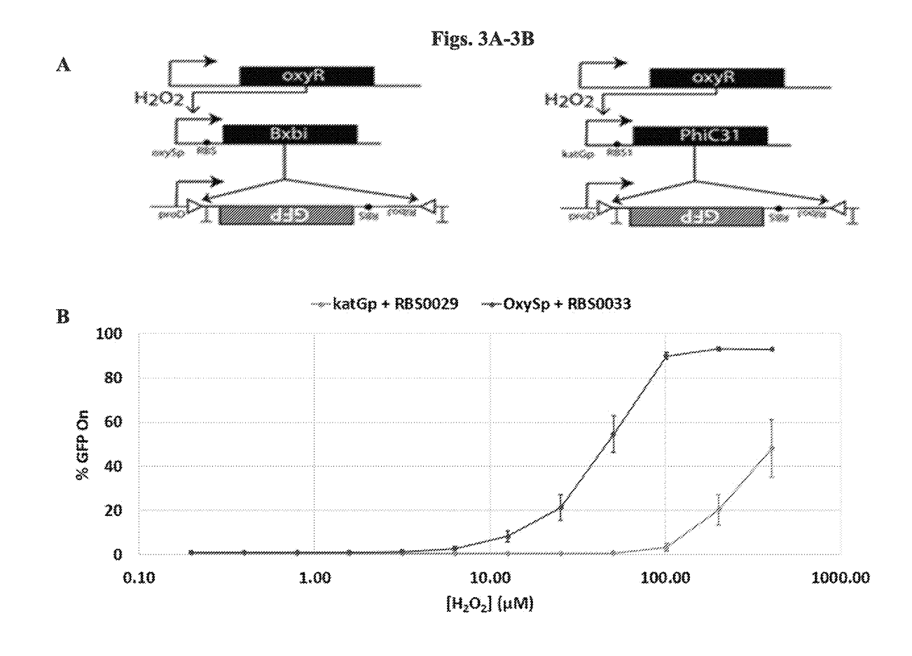

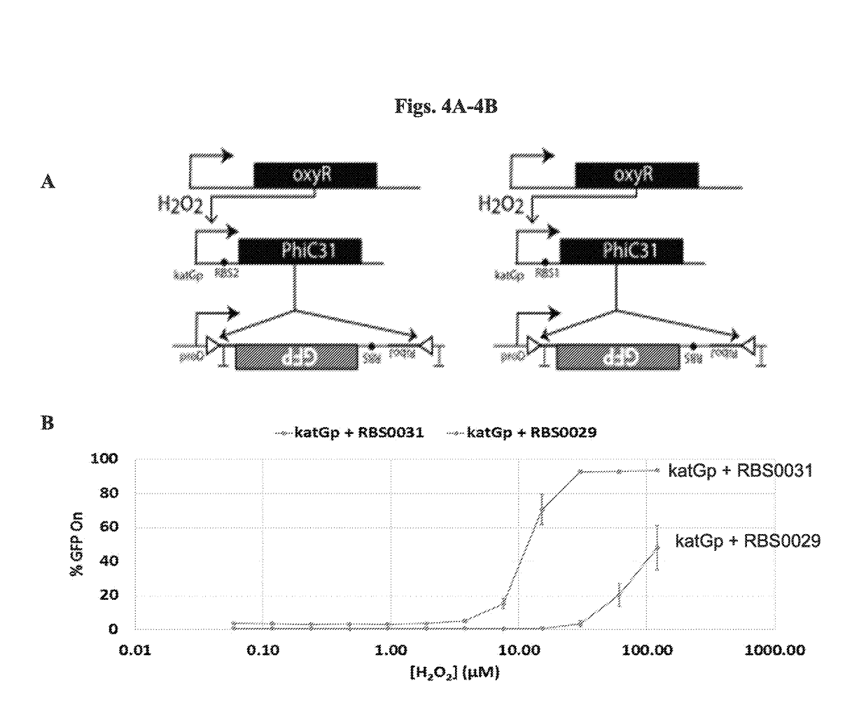

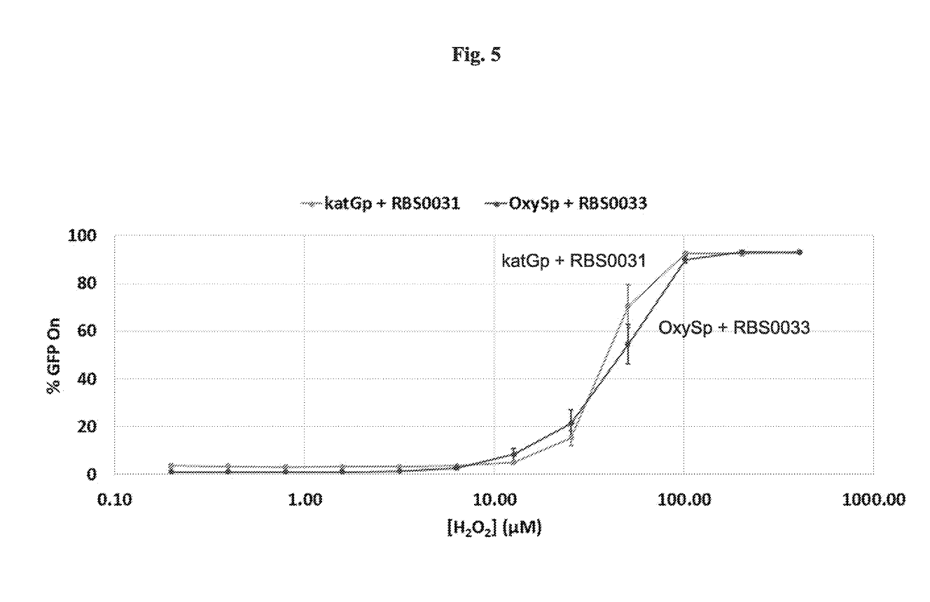

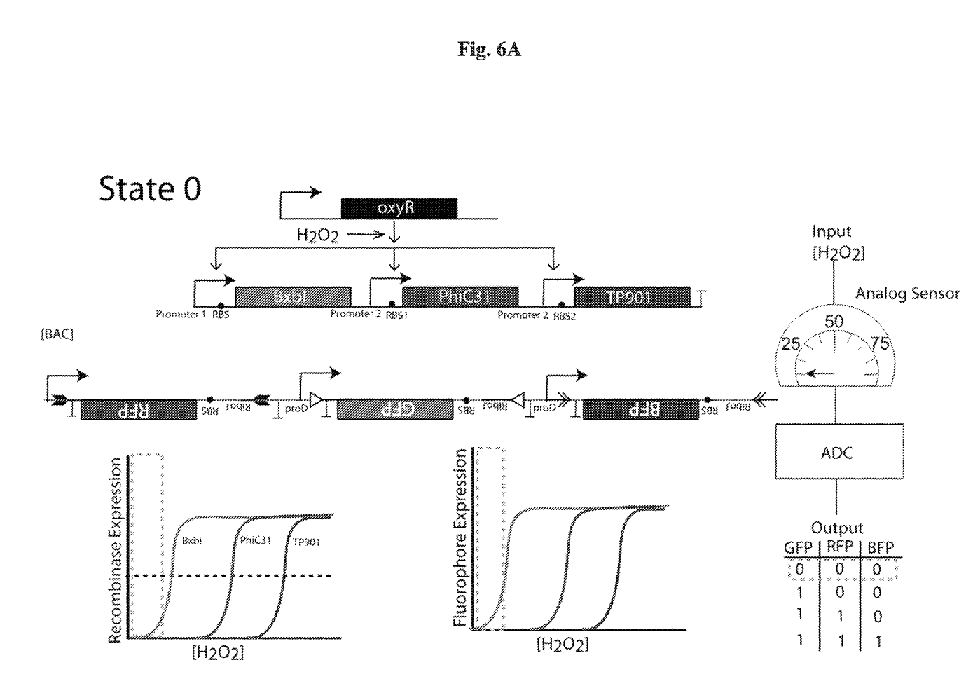

When used in a biological analog signal processing circuit, as shown in FIG. 3 and FIG. 4, combinations of different promoters and different ribosome binding sites can be used to tune expression of an output molecule in response to varying concentrations of input molecule (e.g. H₂O₂). For example, FIG. 3 demonstrates that combinations of different promoters and different ribosomal binding sites (RBS) in two circuits results in distinct output molecule (GFP) expression profiles. FIG. 4 demonstrates the same promoter and different ribosomal binding sites in two circuits results in distinct output molecule (GFP) expression profiles. FIG. 5 demonstrates that expression of output molecules from two different circuits can be tuned to have similar sensitivity to input molecules by using different combinations of promoters and ribosomal binding sites.

### Example 3: A Biological Analog Signal Processing Circuit as a Three-Concentration Classifier

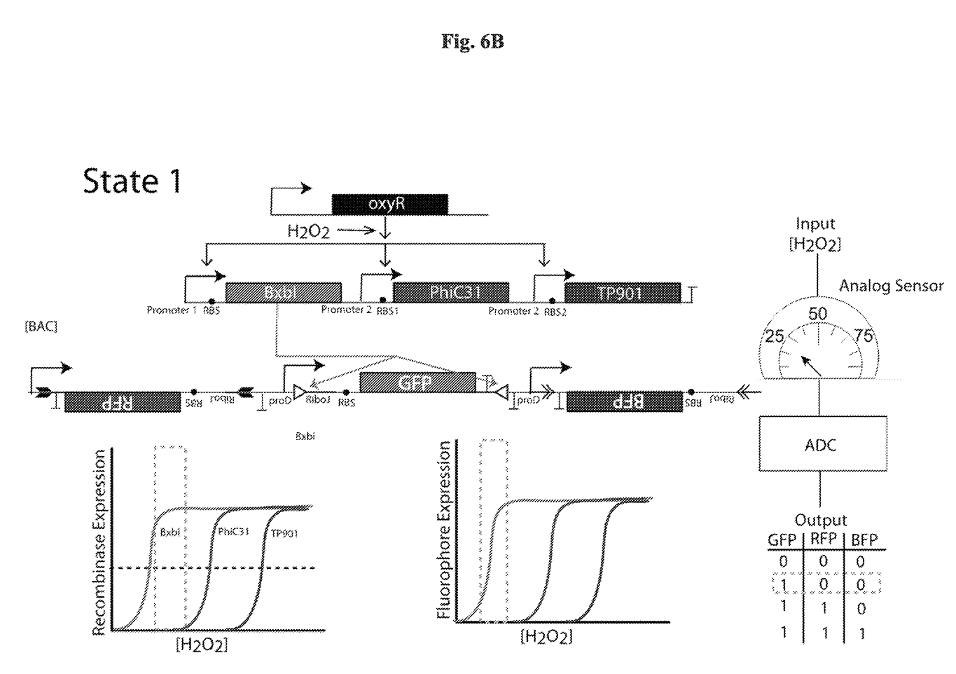

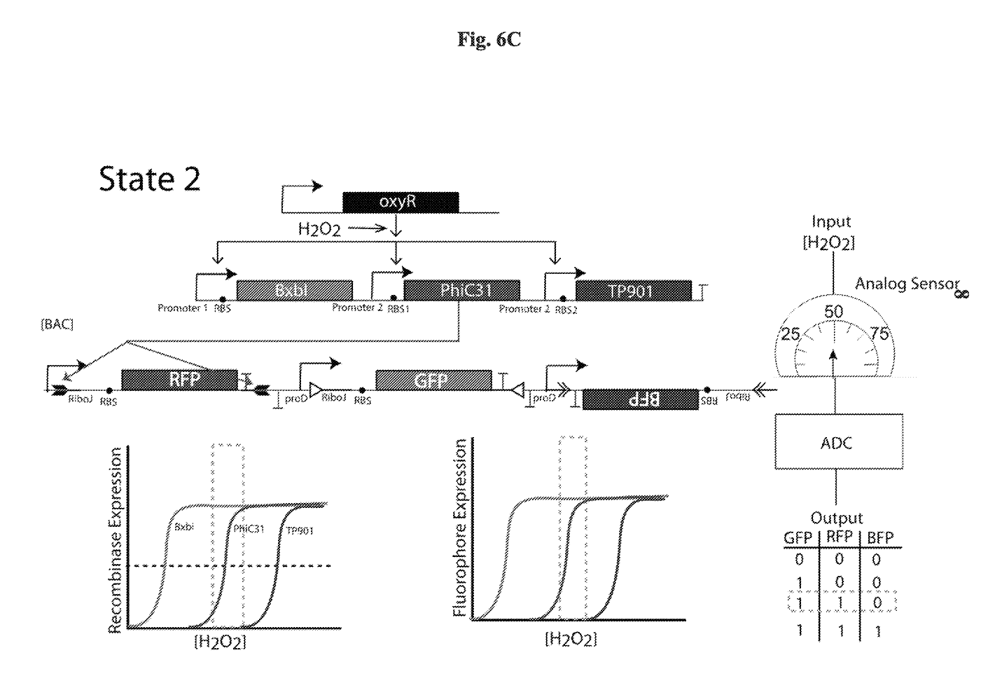

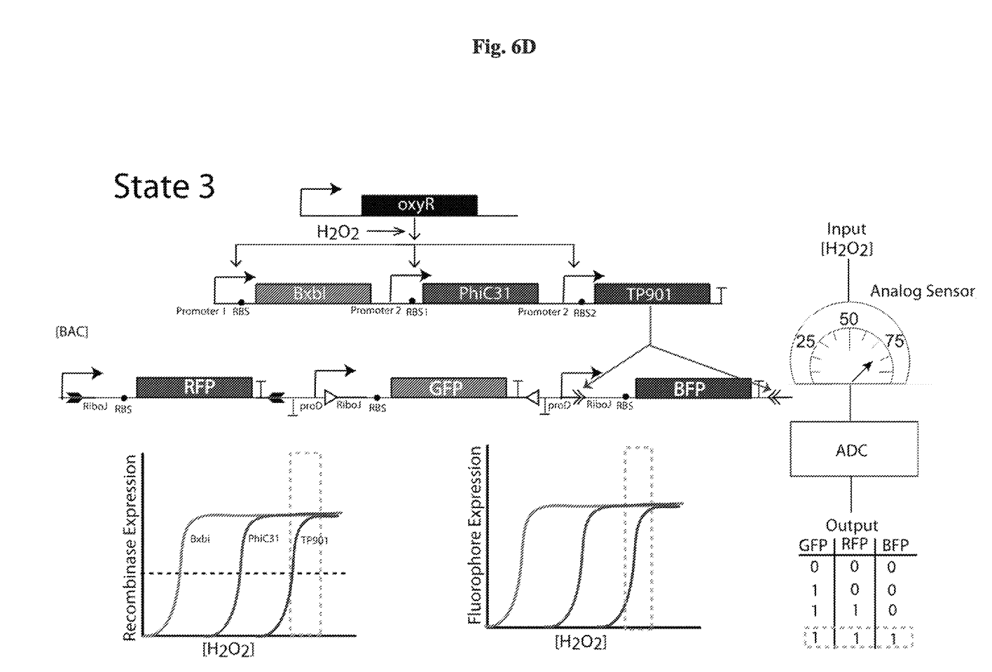

This example demonstrates control of expression of three genes at different concentrations of an input molecule. The circuit discussed in this example is depicted in FIGS. 6A-6D. In this design, the oxyR transcription factor is constitutively produced. It senses H₂O₂ and activates promoters (oxySp or katGp), here denoted as promoter 1 and promoter 2 with different affinities. These promoters control the transcription of a recombinase (BxbI, PhiC31, or TP901). Depending on the RBS strength, the translation efficiency of the recombinase is different. As a result, the combination of different promoters and translation strengths alters the concentration of H₂O₂ necessary for activation for the recombinases. These recombinases then “flip” on the expression of their outputs: GFP, RFP, or BFP.

In State 0 of this example, shown in FIG. 6A, a very low concentration of H₂O₂ results in no expression of output molecules. In State 1 of this example, shown in FIG. 6B, a moderate concentration of H₂O₂ results in the activation of promoter 1 and expression of GFP. At a higher concentration of H₂O₂, shown in FIG. 6C, the circuit reaches State 2 and promoters 1 and 2 are activated resulting in the expression of GFP and RFP. In State 3, at even higher concentrations of H₂O₂, shown in FIG. 6D, promoters 1 and 2 remain active and RBS2 (which has a lower translation efficiency than RBS1) is activated, resulting in the expression of GFP, RFP and BFP.

### Example 4: A Biological Bandpass Filter

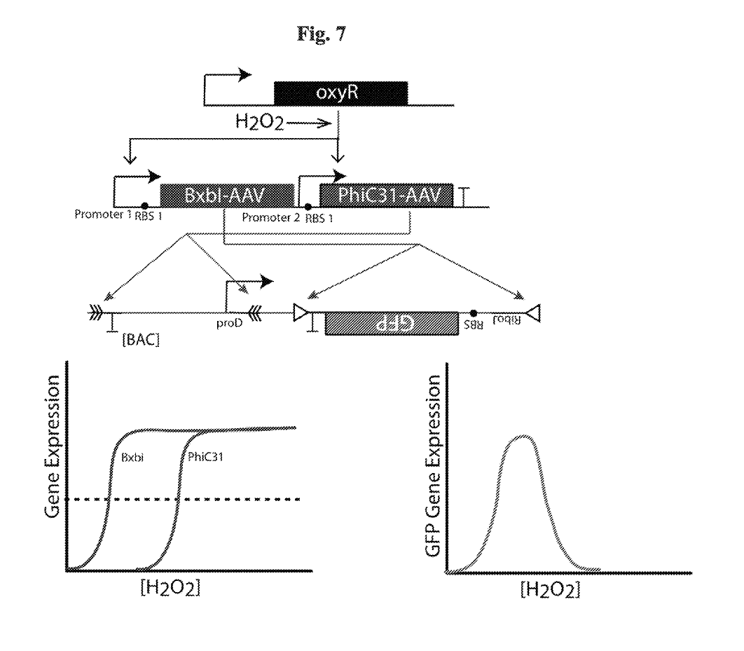

In this example, the design of a biological bandpass filter is disclosed. The circuit discussed in this example is depicted in FIG. 7. The oxyR transcription factor is constitutively produced. It senses H₂O₂ and activates promoters (e.g., oxySp or katGp), here denoted as promoter 1 and promoter 2 with different affinities. These promoters control the transcription of a recombinase (e.g., BxbI or PhiC31). There are degradation tags attached in this figure, but they are not essential). As discussed above, the combination of different promoters alters the concentration of H₂O₂ necessary for activation for the recombinases.

Upon activation of their respective promoters and subsequent expression, the recombinases “flip” different circuit elements. BxbI flips GFP “ON” (GFP is expressed), and PhiC31 flips the promoter that is operably linked to GFP “OFF” (expression of GFP is inactivated). The BxbI recombinase has a lower threshold concentration of H₂O₂ than the PhiC31 recombinase due to a difference in the affinity to the promoters. In this circuit, GFP is turned on at medium concentrations of H₂O₂. However, at higher concentrations of H₂O₂, PhiC31 is turned “ON”, and it flips the promoter that is linked to BxbI, thus turning GFP “OFF”. The cumulative effect is a bandpass filter for intermediate concentrations of hydrogen peroxide.

### Example 5: A Biological Analog Signal Processing Circuit as a 2-Bit Analog to Digital Converter

The circuit described in this example is depicted in FIGS. 8A-8D. This design builds off the bandpass filter described in Example 4. In State 0 (FIG. 8A), where there is a low concentration of H₂O₂, there is no expression of output molecules (e.g., mCherry or GFP). In State 1 (FIG. 8B), a moderate concentration of H₂O₂ activates expression of BxbI, which results in flipping “ON” GFP expression. In State 2 (FIG. 8C), the promoter that was previously flipped by PhiC31 and turned GFP “OFF” now also simultaneously turns mCherry “ON,” because after being flipped by PhiC31, the promoter now faces in the proper direction to activate transcription of mCherry. Therefore at a higher than moderate concentration of H₂O₂, RFP is expressed and GFP is not expressed. In State 3 (FIG. 8D), a third recombinase that is turned “ON” at the highest concentrations of H₂O₂ has been activated. When the threshold for this high concentration is reached, a second promoter flips and turns GFP back “ON”. This flipping event does not affect mCherry expression. Therefore, at the highest concentration, both GFP and mCherry are expressed.

### Example 6: Converting Analog Input Concentration to Discrete Digital Gene Expression Regimes

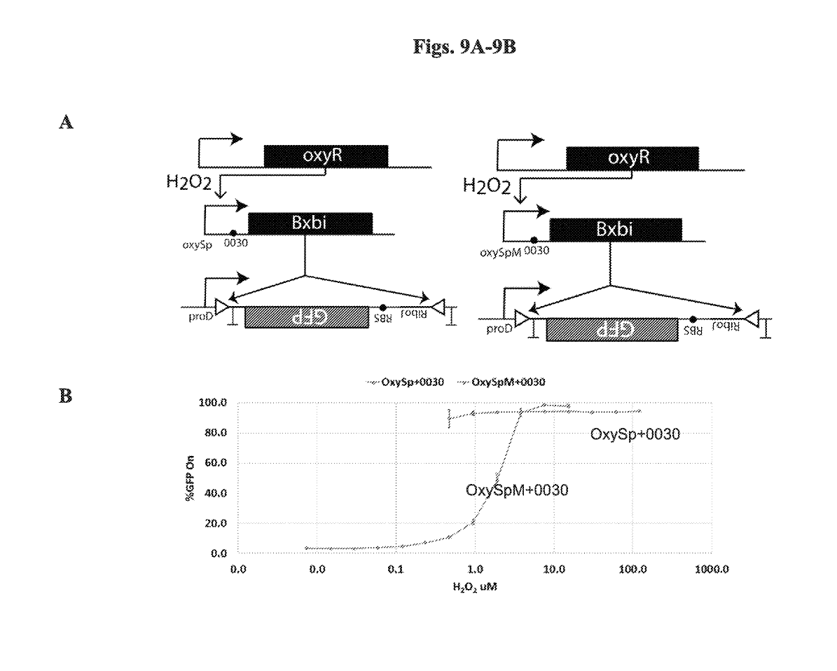

The circuit described in this example is depicted in FIG. 9A and FIG. 9B. By changing the promoter strength (and thus transcription rate of the recombinase), digital switches are obtained (with regards to input H₂O₂) at different concentrations.

Strains and Plasmids: the circuits realized in this work (see FIG. 9A) were prepared with basic molecular cloning techniques. In particular the sensor was inserted in a low copy plasmid pSC101 while the reporter plasmid was inserted in BAC vector which copy number is under arabinose control. In order to amplify the difference between “on” and “off” cells only for the assay and not during the sensing, thus reducing the energetic burden. The bacterial host used for the characterization was E. coli EPI300.
Circuit characterization: the sensor and the reporter were freshly transformed every time into the host, in order to avoid spurious recombinase activation. From the plates, cultures were grown at 37° C. in 5 ml of Azure Hi-Def media with appropriate antibiotics for 18h. The culture were then diluted 1:2500 and grown again for 20 min and eventually moved to a 96 well plate (200 μl) where they were induced with H₂O₂ with concentrations comprised between 121-0.06 μM (with a 2 fold dilution at every well). The induced cell were then incubated for 20h at 30° C. The cells were then spun down and washed with PBS (250 μl) to remove the remaining H₂O₂ and resuspended in fresh media with copy control and then grown for 10h at 30° C. Cell were then diluted 25× in PBS and assayed using BD LSRFortessa. A minimum of 50000 events were collected for each sample, the data were then analyzed using FlowJo software.

The oxyR transcription factor is constitutively produced. It senses H₂O₂ and activates the oxySp or oxySpM promoter (see FIG. 9A, left or right schematic, respectively). These promoters differ in mutations that change the affinity of RNA polymerase for the promoter, and thus the strength of the promoter. The mutations are in the −10 region of the promoter, and the promoters are otherwise the same (same binding affinity for oxyR). The RBS is the same. The promoters controls the transcription of a recombinase (bxbi). Depending on the promoter strength, the transcription efficiency of the recombinase is different. As a result, the concentration of H₂O₂ necessary for activation is different. These recombinases then “flip” on GFP expression.

In the data presented in FIG. 9B, the percent of cells in a population that have turned ON GFP expression in response to different concentrations of H₂O₂ are tracked. The weaker promoter switches off at a low concentration of H₂O₂, whereas the stronger promoter did not switch off in the range of H₂O₂ tested.

### Example 7: Analog Sensor for the Reactive Oxygen Species Hydrogen Peroxide (H2O2)

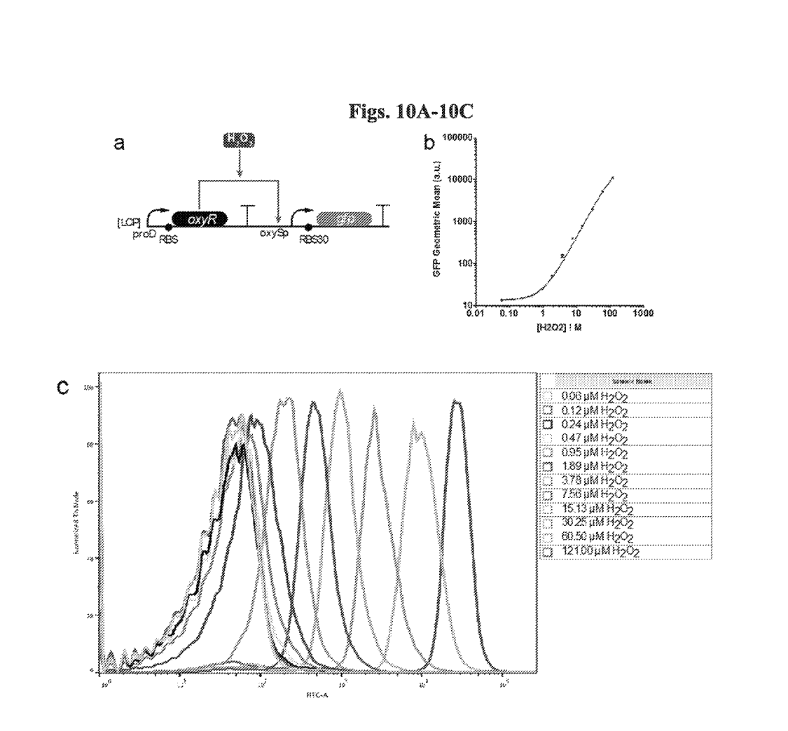

An analog sensor was first created for the reactive oxygen species hydrogen peroxide (H₂O₂). H₂O₂ plays intricate biological roles across all kingdoms of life, and its regulation is linked to human health and disease. H₂O₂ oxidizes and activates the E. coli transcription factor OxyR. OxyR was constitutively expressed to set a minimum concentration of OxyR in the cell, since genomically expressed oxyR is auto-negatively regulated, and gfp was placed under the control of the OxyR-regulated oxyS promoter (oxySp) on the same low copy plasmid (LCP) (FIG. 10). GFP expression was continuously increased by H₂O₂ over more than two orders of magnitude of concentration, indicating that OxyR is a wide-dynamic-range analog sensor for H₂O₂ in this context.

### Example 8: Genetic Comparators

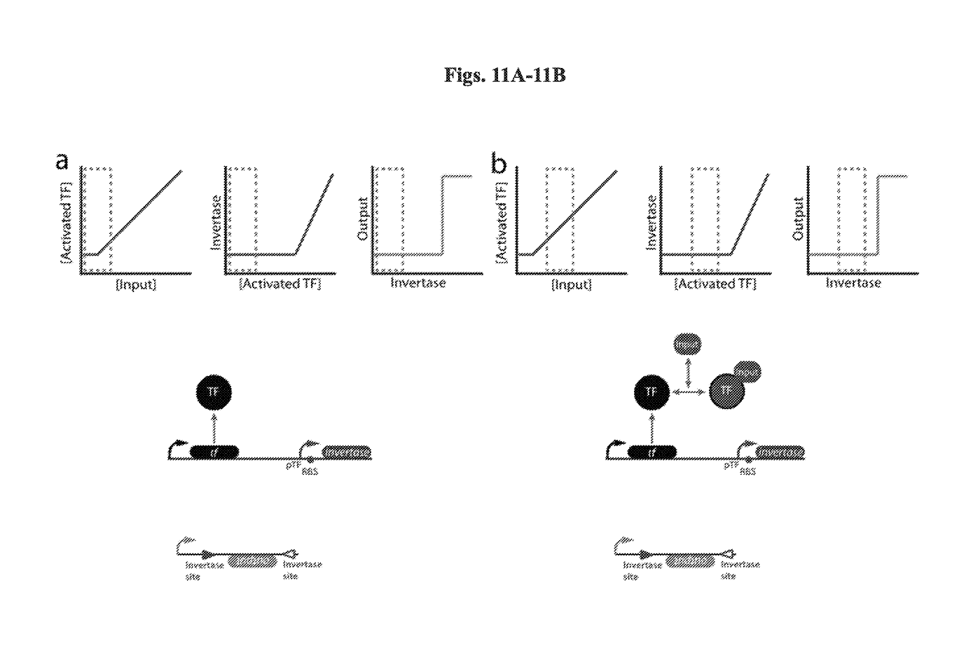

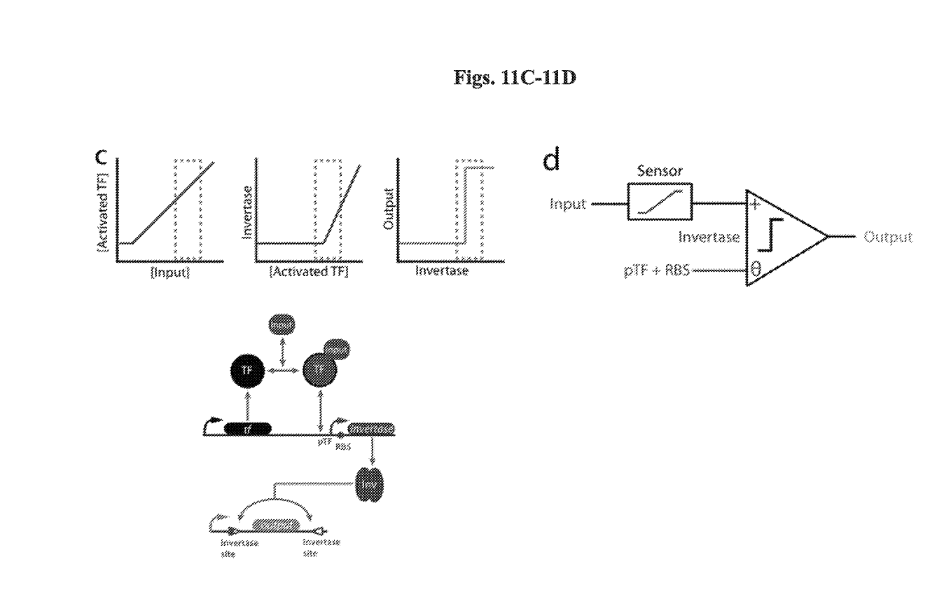

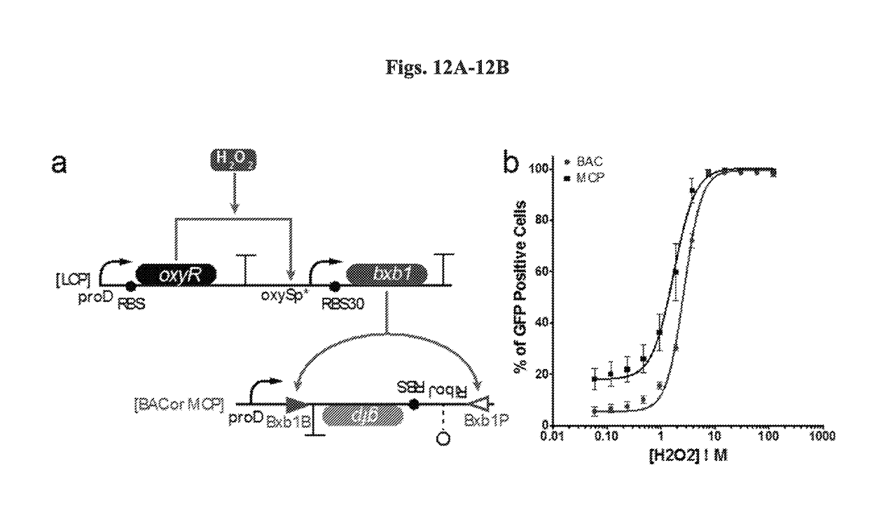

Genetic comparators (FIG. 11) were created next, which can be conceptualized as composed of three components. The first component is the threshold module. It includes a promoter, which is regulated by the transcription factor, and a ribosome binding site (RBS) that together set the expression level of the downstream recombinase gene and determine the threshold for comparator activation. This is in contrast to electronic comparators, where a second input can dynamically set the threshold. The second module is the digitization module, which is composed of a recombinase whose expression is controlled by the threshold module. The recombinase digitizes the input value by inverting the orientation of a targeted DNA segment maintained at a very low copy number. The third module is the DNA that is inverted by the recombinase, which can contain a gene or gene-regulatory elements, such as a transcriptional promoters or terminators, to alter expression of the desired output(s).

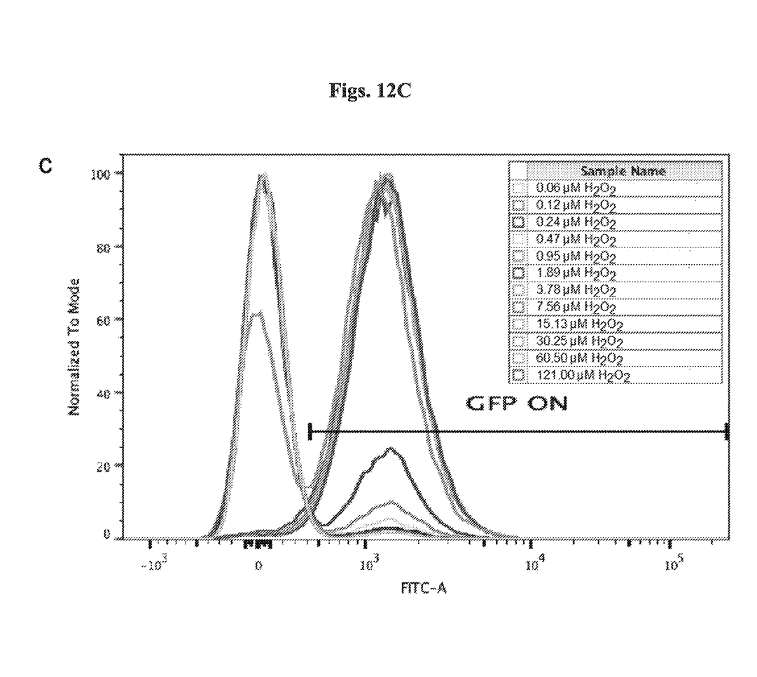

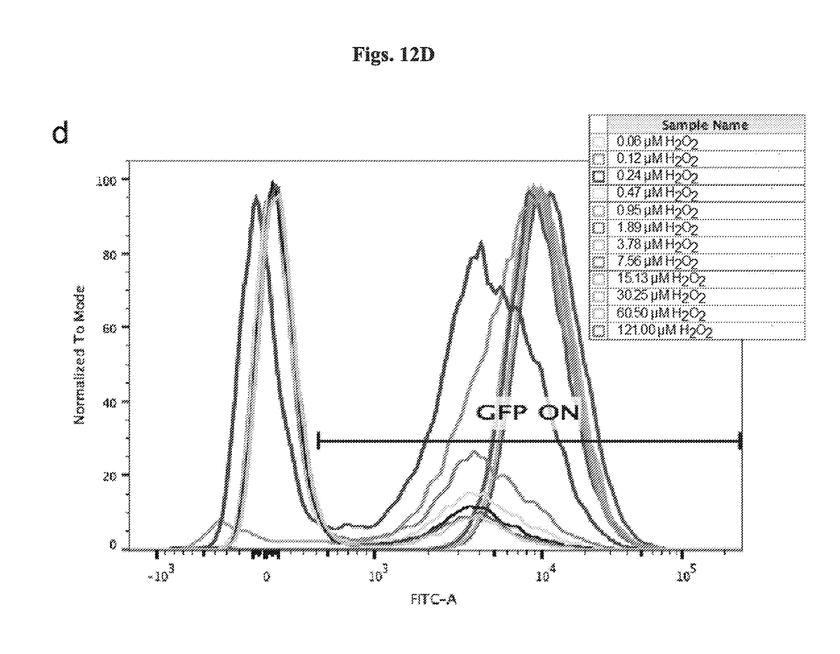

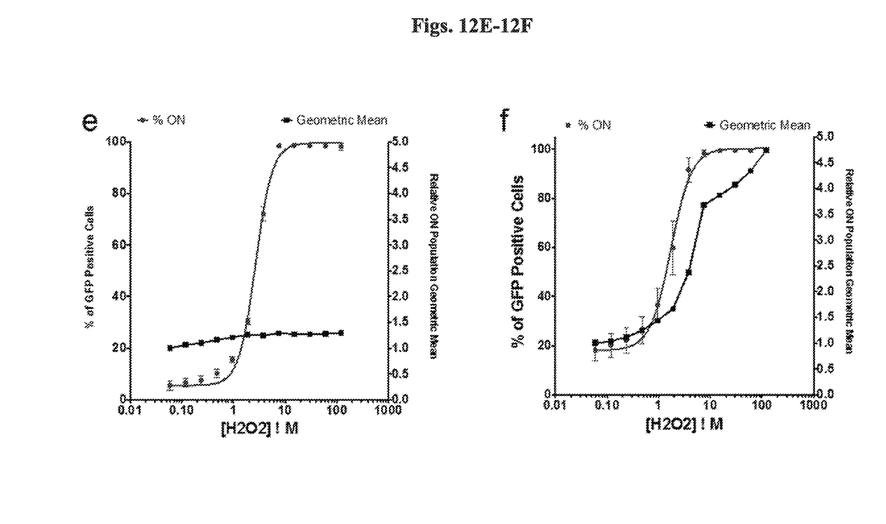

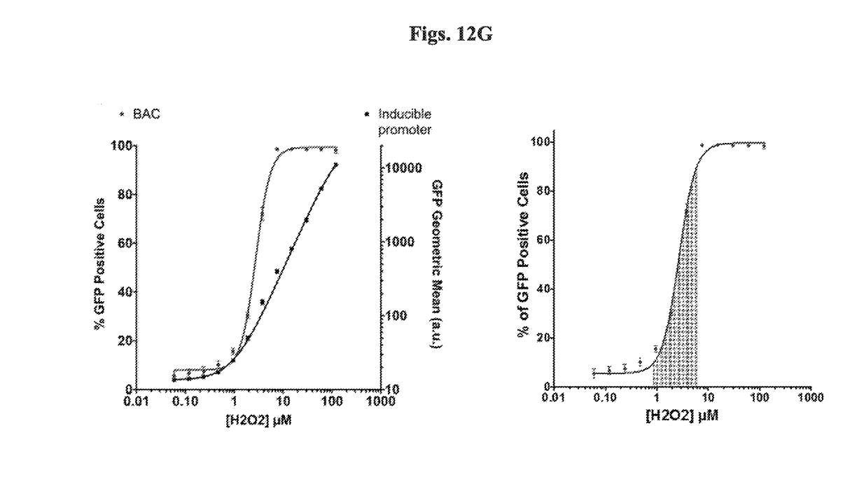

The digitization aspect of the comparator relies on recombinases, and thus how the number of sites targeted by recombinases affects signal digitization into two distinct gene expression states within individual cells was explored. The serine integrases (recombinases) we used flip, excise, or integrate DNA depending on the orientation of attB and attP recombinase-recognition sites, and their activity is unidirectional unless co-factors are present. Recombinases have been used to build digital counters, integrate logic and memory, and amplify input-output transfer functions. To discretize H₂O₂ input levels, the Bxb1 recombinase was placed under the control of the oxySp promoter on a LCP. In order to keep the basal level of bxb1 minimal such that there is little recombinase activity in the cell in the uninduced state, a ClpXP-mediated degradation tag was added to the 3′ end of the bxb1 coding sequence (FIG. 12A). Two options were tested as reporters for recombinase activity: a medium copy plasmid (MCP, maintained at 20-30 copies per cell) and a bacterial artificial chromosome (BAC, maintained at 1-2 copies per cell), each of which contained a constitutive promoter upstream of an inverted gfp gene flanked by oppositely oriented attB and attP sites.

bxb1 expression was induced at different concentrations of H₂O₂ and GFP expression was measured via flow cytometry (FIGS. 12B-12D). A threshold for calling cells GFP “ON” or “OFF” was set and this threshold was used to calculate the percent of cells that were ON (% ON) at each concentration of H₂O₂ (see section entitled “Data Processing and Calculations”). The % ON vs. H₂O₂ concentration data was fit to a sigmoidal function to generate input-output transfer functions. The MCP and BAC reporters had similar transfer functions, although cells using the MCP reporter had a higher percent of cells ON at the basal H₂O₂ concentration (FIG. 12B). However, GFP expression in cells with the MCP reporter exhibited a multi-modal distribution especially at intermediate concentrations of H₂O₂, which suggests partial plasmid flipping and thus mixed GFP expression levels in different cells (FIG. 12D). This effect was further demonstrated by increases in the geometric mean of GFP levels with increasing H₂O₂ in the ON population (FIG. 12F). In contrast, cells with the BAC reporter only exhibited a bi-modal distribution (FIG. 12C), and the geometric mean of the ON population only marginally increased with H₂O₂ concentration (FIG. 12E). Thus, the BAC reporter converts the input concentration of H₂O₂ into digital OFF and ON gene expression states within individual cells better than the MCP reporter.

### Data Processing and Calculations

Calculating the Sigmoidal Fit, Input Threshold, and Relative Input Range

The data from the BAC circuit in FIGS. 12A-12F are shown as an example.

1. Calculate % of cells at each concentration of H₂O₂ that fall within the “GFP ON” gate. The “GFP ON” gate is drawn for each experiment at the FITC fluorescence level where the fluorescence distribution of uninduced cells intersects with the fluorescence distribution of induced cells, or in between the uninduced and induced distributions when the fluorescence distributions are well-resolved and do not overlap. Take the average % ON of biological replicates to calculate the mean and standard deviation (FIG. 12C).

2. To derive the transfer function, fit the mean % ON vs. H₂O₂ concentration data to a Hill-like sigmoidal function (FIG. 12G, gray (top) solid line):

$$\% ON = {ON}_{Max} \frac{{[ {H}_{2} {O}_{2} ]}^{n}}{{[ {H}_{2} {O}_{2} ]}^{n} + {( {K}_{on} )}^{n}} + {ON}_{Min}$$

Where [H₂O₂] is the independent variable, ON_{Min} is the empirically observed minimum percent ON, and ON_{Max}, Kₒₙ, and n are fit to the data.

3. The input dynamic range is defined as the input H₂O₂ concentration span that yields 10% ON to 90% ON, as interpolated from the transfer function: see FIG. 12G (right panel).

4. Calculate the relative input range from the 10% ON and 90% ON input values:

$$Relative Input Range ( RIR ) = \frac{{[ {H}_{2} {O}_{2} ]}_{90 \%}}{{[ {H}_{2} {O}_{2} ]}_{10 \%}}$$

Calculating the Fit to a Bandpass Filter Circuit

The fit to a bandpass filter circuit was derived by subtracting the transfer function of the low-pass comparator from the transfer function of the high-pass comparator:

$$\% ON = {ON}_{Max , hp} \frac{{[ {H}_{2} {O}_{2} ]}^{n , hp}}{{[ {H}_{2} {O}_{2} ]}^{n , hp} + {( {K}_{on , hp} )}^{n , hp}} + {ON}_{Min , hp} - {ON}_{Max , l p} \frac{{[ {H}_{2} {O}_{2} ]}^{n , lp}}{{[ {H}_{2} {O}_{2} ]}^{n , lp} + {( {K}_{on , hp} )}^{n , lp}}$$

Where hp subscript denotes a variable from the “high pass” circuit and 1p subscript denotes a variable from the “low pass” circuit.

Calculating the Relative Resolution of a Genetic Analog-to-Digital Converter Circuit

The relative resolution (RQ) was defined as:

$$RQ = \frac{ADC RIR}{{2}^{bits} - 2}$$

### Where the ADC RIR is:

$$\frac{{[ {H}_{2} {O}_{2} ]}_{50 \% , high}}{{[ {H}_{2} {O}_{2} ]}_{50 \% , low}}$$

[H₁O₂]_{50%, high} is the concentration of H₂O₂ necessary for 50% of cells to turn ON for the highest threshold comparator in the ADC, and [H₂O₂]_{50%, low} is the concentration of H₂O₂ necessary for 50% of cells to turn ON for the lowest threshold comparator in the ADC.

The number of bits is the total number of bits encoded by the ADC. 2 is subtracted in the denominator because 2 of the states are encoded outside of the ADC RIR (e.g., below the [H₂O₂]_{50%, low} concentration and above the [H₂O₂]_{50%, high} concentration, states 000 and 111).

### Example 9: Analog-to-Digital Comparator Circuits for Transactivation

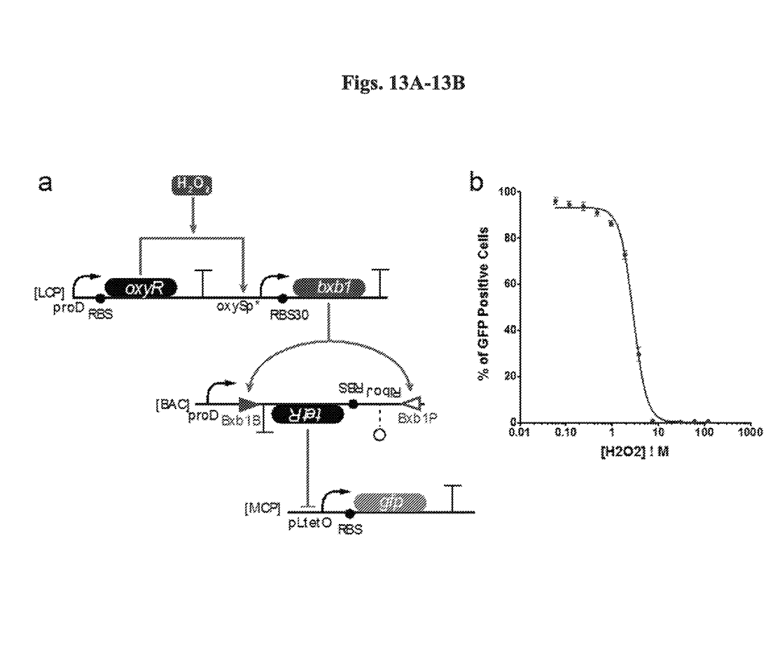

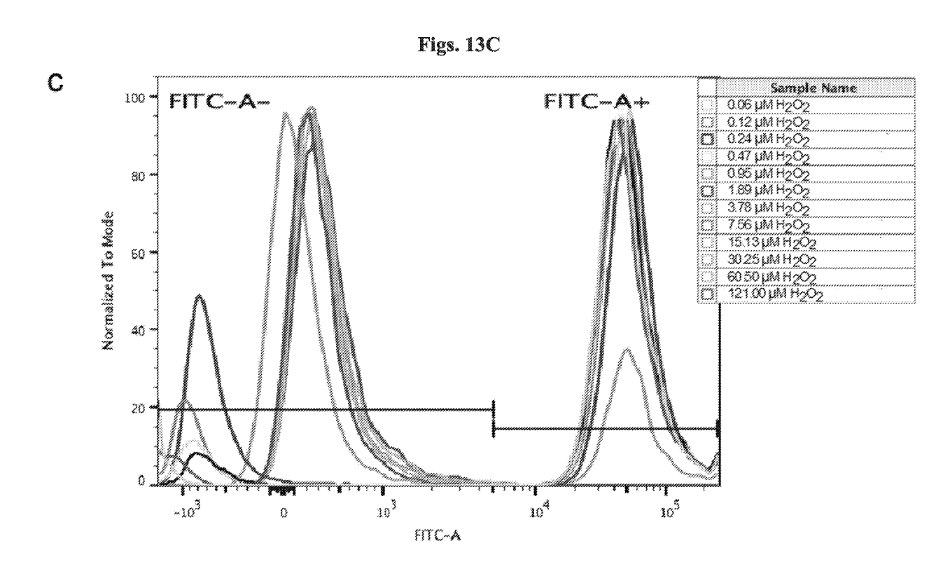

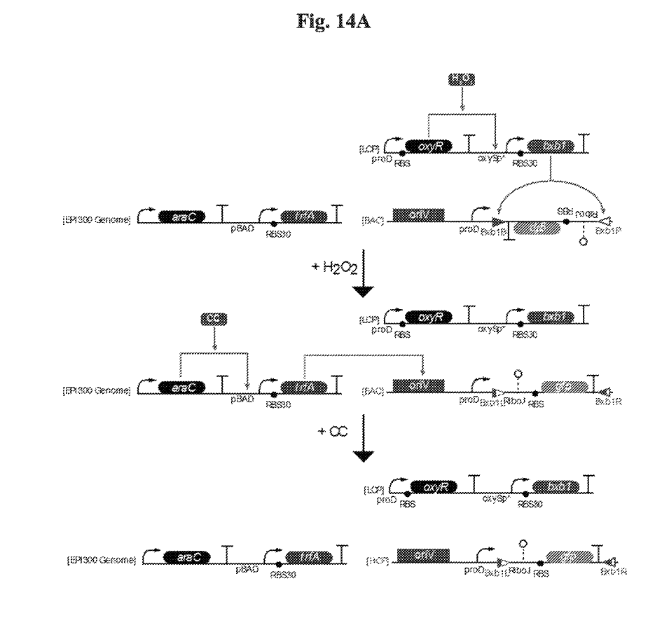

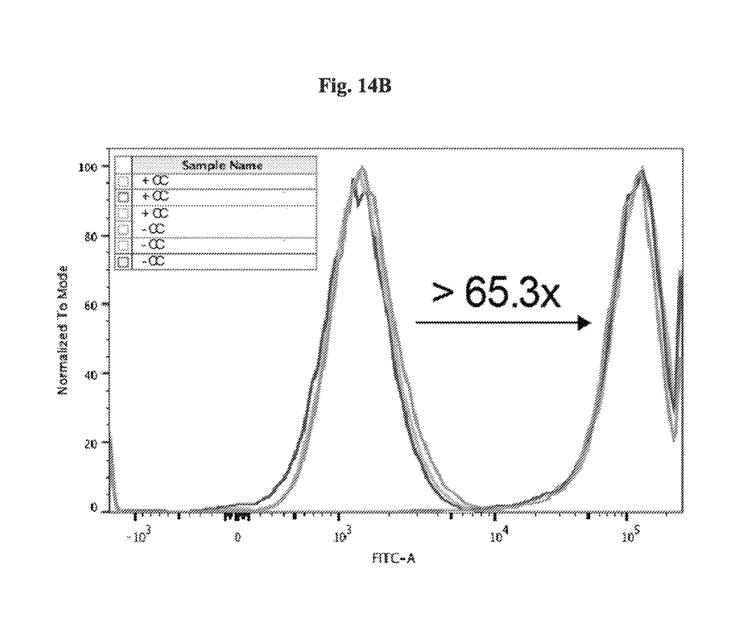

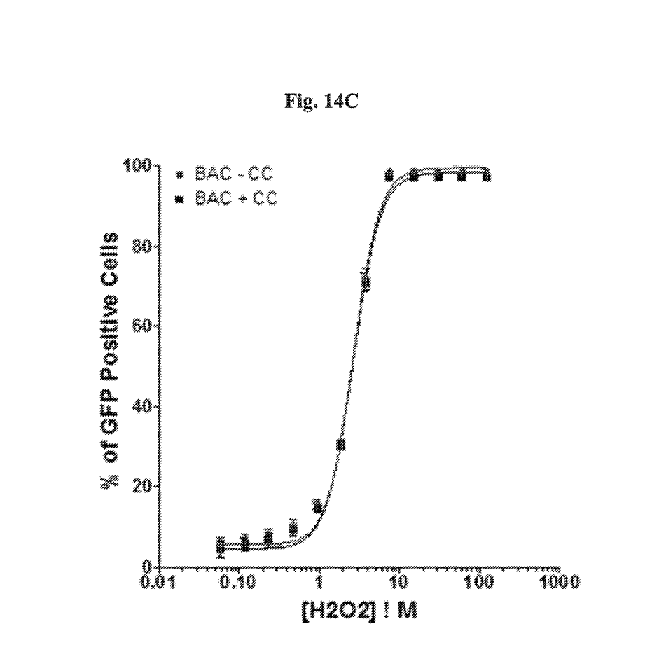

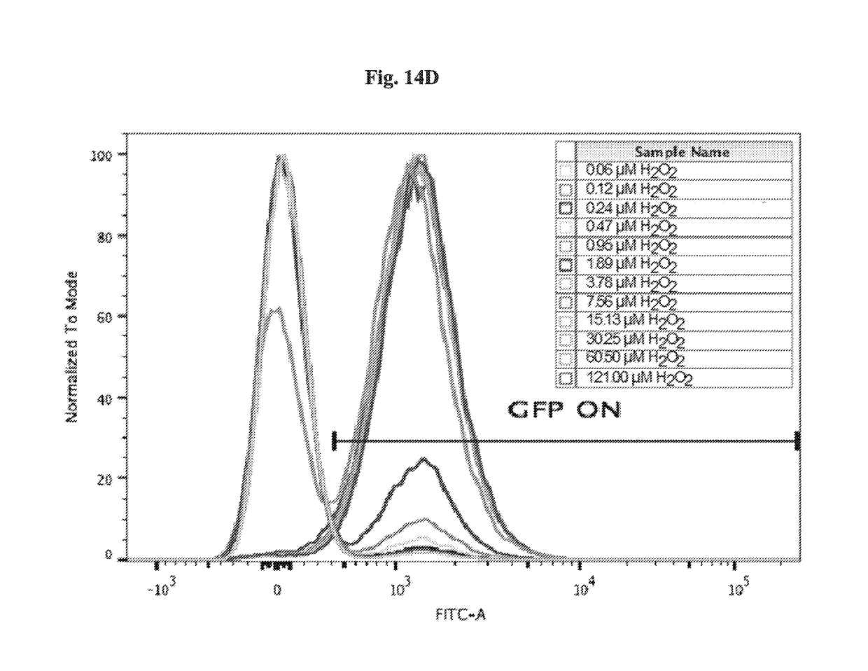

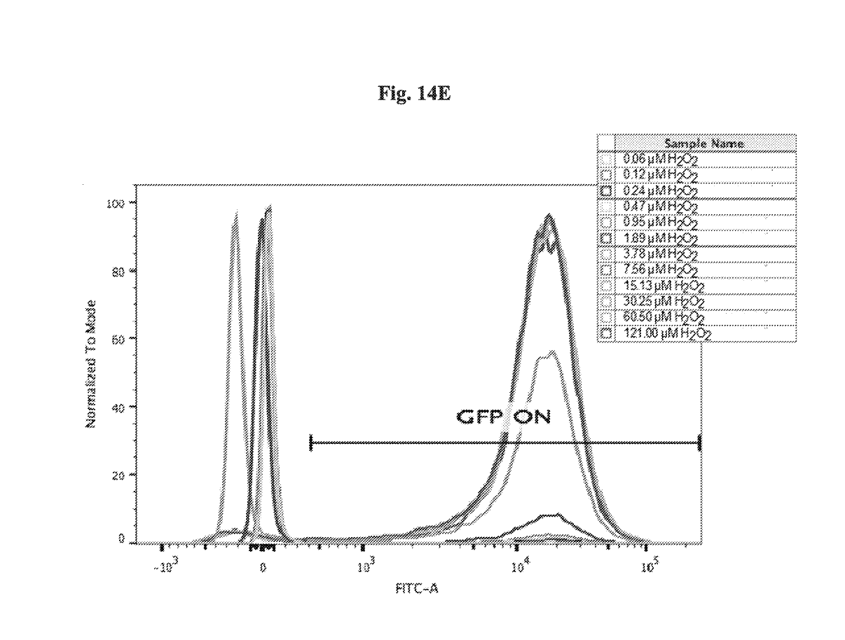

An experiment was designed to demonstrate that the analog-to-digital comparator circuits could be used to drive downstream circuits in a trans-acting fashion. To construct a cascade, gfp was replaced in the BAC expression operon with tetR and placed gfp under the control of the TetR-regulated promoter pLtetO on a MCP (FIGS. 13A-13C). In the absence of H₂O₂, the majority of cells expressed gfp and were in the ON state. In the presence of H₂O₂, gfp expression from pLtetO was efficiently repressed and the majority of cells were switched into an OFF state. These results demonstrate that recombinase circuits can be used together with trans-acting regulation to assemble functional cascades. A method was also developed to simplify the quantification of OFF versus ON since fluorescent gene expression levels from the BAC are low and can result in overlapping OFF and ON gene expression distributions in flow cytometry. This method amplifies the copy number of the reporter from low to high but preserves the bi-modal nature of the OFF and ON populations, thus confirming the digital flipping of the BAC (FIGS. 14A-14E).

### Example 10: Varying Comparator Thresholds and Transition Bands

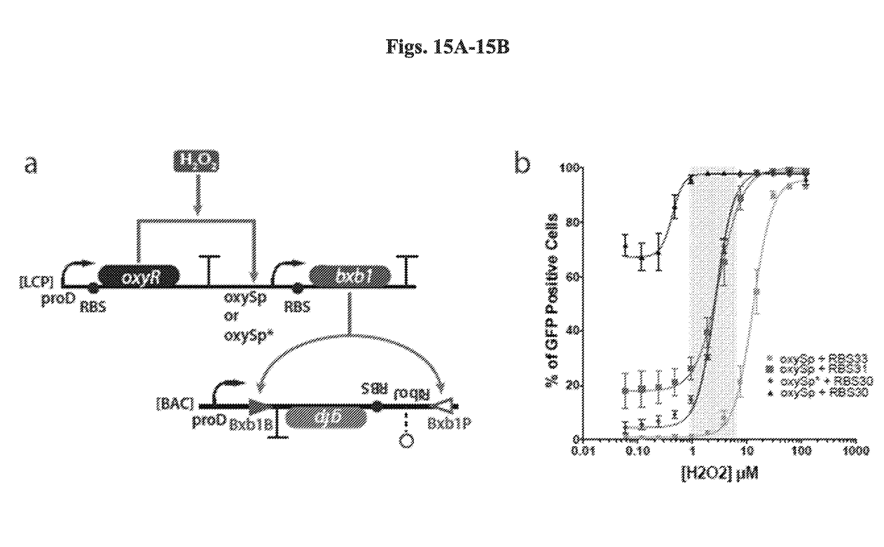

The threshold module of the comparator can be used to shift the discretization threshold. Comparators with different thresholds and transition bands were created (e.g., the input dynamic range) by assembling combinations of promoters with different transcription-factor affinities, ribosome binding sites, and recombinases (FIGS. 15A-15B). The transition band was defined as the range of H₂O₂ concentrations across which the percent of cells expressing the output fluorophore is between 10% and 90% as interpolated from the transfer function (though on a single cell level, gene expression is binary), and the “relative input range” of the transition band was calculated to define its width (see above section entitled “Data Processing and Calculations”). A narrow relative input range is indicative of low variability across the cell population around the input threshold for state switching, which is important for robustness to noise.

The low-threshold comparator used the Bxb1 recombinase and the oxySp promoter, which is activated at low H₂O₂ concentrations. Different RBSs were screened in this construct and none of these circuits turned ON below 1 μM H₂O₂ without also exhibiting a high basal level of recombinase activity (FIG. 15A). To address this issue and reduce basal bxb1 expression, a strong RBS (RBS30) was used and the −10 region of the oxySp promoter was randomly mutated to create a low-threshold comparator that had a transition band between 0.91-6.44 μM H₂O₂, giving it a relative input range of 7.10 (FIG. 15B, FIG. 11A). To create a medium-threshold comparator, different RBSs controlling phiC31 recombinase translation from the katGp promoter were tested. A circuit with RBS31 had a transition band of 6.50-25.13 μM, which is a relative input range of 3.87 (FIG. 11B). To create a high-threshold comparator, tp901 recombinase was used and different RBS and promoter combinations were screened. The ahpCp promoter-recombinase combination had an intermediate activation threshold. The katGp promoter was used to test different RBSs. Using RBS33 yielded a circuit with improved behavior, with a transition band of 15.19-85.49 μM H₂O₂ and relative input range of 5.63 (FIG. 11C).

Thus, examples of genetic comparators with different activation thresholds are provided herein. In one example of the low-threshold H₂O₂ comparator circuit, OxyR is constitutively expressed from a low-copy plasmid (LCP) and activates transcription of bxb1 recombinase from either the oxySp or oxySp* promoter on the same LCP in response to H₂O₂. Bxb1 translation is altered by the strength of the ribosome binding site (RBS). Bxb1 inverts the gfp expression cassette located between inversely oriented attB and attP sites (triangles) on a bacterial artificial chromosome (BAC), thus turning on GFP expression. The gfp cassette has a ribozyme sequence for cleaving the 5′ untranslated region of an mRNA transcript (RiboJ)58, a computationally designed RBS59, the gfp coding sequence, and a transcriptional terminator. The percent of GFP positive cells at different H₂O₂ concentrations are measured by flow cytometry. Different combinations of oxySp and oxySp* promoters and RBSs exhibit different H₂O₂ thresholds and basal levels for GFP activation. The oxySp* and RBS30 combination (diamonds) had the lowest threshold and a narrow transition band (shaded region). In one example, the medium-threshold H2O2 comparator circuit is the same as the low-threshold H₂O₂ comparator circuit described above, except with the katGp promoter instead of the oxySp or oxySp* promoters, and phiC31 recombinase and att inversion sites instead of bxb1 recombinase and att inversion sites. Different combinations of the katGp promoter and RBSs had different H₂O₂ thresholds and basal levels for GFP activation. The katGp and RBS31 combination had a medium H₂O₂ threshold and narrow transition band (shaded region). In one example, the high-threshold H₂O₂ comparator circuit is the same as the low-threshold H₂O₂ comparator circuit described above, except with either the katGp promoter or ahpCp promoter instead of the oxySp or oxySp* promoters, and tp901 recombinase and att inversion sites instead of bxb1 recombinase and att inversion sites. Different combinations of katGp and ahpCp promoters and RBSs exhibited different H₂O₂ thresholds for GFP activation. The katGp and RBS33 combination had the highest threshold and a narrow transition band. (See section entitled “Data Processing and Calculations”.) The errors (standard deviation) are derived from flow cytometry experiments of three biological replicates, each of which involved n>30,000 gated events. Representative flow cytometry histograms show GFP expression for the low threshold circuit described above with oxySp* and RBS30 (data not shown). Representative flow cytometry histograms also show GFP expression for the medium threshold circuit with katGp and RBS31 (data not shown). Representative flow cytometry histograms further show GFP expression for the high threshold circuit with katGp and RBS33 (data not shown).

### Example 11: Building Complex Signal-Processing Circuits in Living Cells

Comparators with different thresholds can be composed together to build more complex signal-processing circuits in living cells. For example, circuits that turn gene expression ON with increasing input concentrations can be considered high-pass circuits (since they allow high-concentration inputs to “pass” or be outputted). Next, to create low-pass circuits (which only allow low-concentration inputs to “pass”), a gene expression cassette that was ON in the basal state was built and an inducible recombinase circuit was used to turn the output gene OFF by inverting the upstream promoter. Then, to create bandpass filters a low-threshold high-pass circuit was combined with either a medium- or high-threshold low-pass circuit, thus implementing logic. The bandpass circuits switched GFP expression ON at low concentrations of H₂O₂ and switched GFP OFF at either medium or high concentrations of H₂O₂, depending on the threshold of the low-pass circuit. The transfer function of each bandpass circuit could be predicted from straightforward addition of the transfer function of the high-pass circuit with the transfer function of the low-pass circuit that composed it (see above section entitled “Data Processing and Calculations”). To determine the transfer functions of the high-pass and low-pass circuits, GFP activation was measured by the comparators using the same reporters for each recombinase. The bandwidth of a bandpass filter was defined as the relative input range over which the circuit switched from 50% ON to 50% OFF. The bandpass circuit composed of the low-threshold high-pass and medium-threshold low-pass had a relative input range of 3.16; the bandpass circuit composed of the low-threshold high-pass and high-threshold low-pass had a wider relative input range of 7.34. This circuit architecture can be adapted to create band-stop filters by making the low-threshold circuit a low-pass and making the high-threshold circuit a high-pass.

Thus, examples of bandpass filters assembled from low-pass and high-pass filters are provided herein. For low-threshold and medium-threshold bandpass filter circuits, OxyR is constitutively expressed and activates transcription of bxb1 and phiC31 in response to H₂O₂. Bxb1 inverts the gfp cassette to enable expression from the upright proD promoter, while PhiC31 inverts the proD promoter to turn off GFP production. Flow cytometry results show the percent of GFP positive cells at different H₂O₂ concentrations for the low-threshold and medium-threshold bandpass filter circuits (data now shown). The transfer functions of the comparators composing the bandpass were characterized to generate the predicted bandpass transfer function, R²=0.75 (data not shown). The low-threshold and high-threshold bandpass filter circuit is the same as the low-threshold and medium-threshold bandpass filter circuit, except RBS33 and tp901 replace RBS31 and phiC31, respectively. An abstraction of bandpass genetic circuits shows that H₂O₂ activates OxyR in an analog fashion. Activated OxyR activates expression of bxb1 and either phiC31 or tp901 depending on the circuit used. The activation threshold is set by the promoters and RBS controlling recombinase expression. The expression of GFP is dependent upon bxb1 expression AND (NOT) phiC31 or tp901 expression. The errors (standard deviation) are derived from flow cytometry experiments of three biological replicates, each of which involved n>30,000 gated events. A circuit was used to characterize the transfer function of the low-threshold comparator that operates as a high-pass in the low-threshold and medium-threshold bandpass filter circuits. OxyR is constitutively expressed from a LCP and activates transcription of bxb1 from the oxySp* promoter and phiC31 from the katGp promoter on the same LCP in response to H₂O₂. Bxb1 inverts the gfp expression cassette on a BAC, turning on GFP expression by pairing it with the proD promoter. The transfer function of the low-threshold comparator that operates as a high-pass in the low-threshold and medium-threshold bandpass filter circuits is a sigmoidal fit to the data (data not shown). This fit was used to generate the high-pass variables in the bandpass function (see section entitled “Data Processing and Calculations”). The errors (standard deviation) are derived from flow cytometry experiments of three biological replicates, each of which involved n>30,000 gated events. For the circuit used to characterize the transfer function of the medium-threshold comparator that operates as a low-pass transfer function in the bandpass circuit in the low-threshold and medium-threshold bandpass filter circuits, the comparator was characterized by turning on GFP expression, rather than turning it off. OxyR is constitutively expressed from a LCP and activates transcription of bxb1 from the oxySp* promoter and phiC31 from the katGp promoter on the same LCP in response to H₂O₂. PhiC31 inverts the gfp cassette on a BAC, turning on GFP expression by pairing it with the proD promoter. The transfer function of the medium-threshold comparator that operates as a low-pass in the bandpass circuit was used to generate the low-pass variables in the bandpass function (see section entitled “Data Processing and Calculations”). The errors (standard deviation) are derived from flow cytometry experiments of three biological replicates, each of which involved n>30,000 gated events. For the circuit used to characterize the transfer function of the low-threshold comparator that operates as a high-pass in the bandpass circuit, OxyR is constitutively expressed from a LCP and activates transcription of bxb1 from the oxySp* promoter and tp901 from the katGp promoter on the same LCP in response to H₂O₂. Bxb1 inverts the gfp cassette on a BAC, turning on GFP expression by pairing it with the proD promoter. The transfer function of the low-threshold comparator that operates as a high-pass in the bandpass circuit was used to generate the high-pass variables in the bandpass function (see section entitled “Data Processing and Calculations”). The errors (standard deviation) are derived from flow cytometry experiments of three biological replicates, each of which involved n>30,000 gated events. A circuit was used to characterize the transfer function of the high-threshold comparator that operates as a low-pass transfer function in the bandpass circuit. The comparator was characterized by turning on GFP expression, rather than turning it off. OxyR is constitutively expressed from a LCP and activates transcription of bxb1 from the oxySp* promoter and tp901 from the katGp promoter on the same LCP in response to H₂O₂. TP901 inverts the gfp cassette on a BAC, turning on GFP expression by pairing it with the proD promoter. The transfer function of the high-threshold comparator that operates as a low-pass in the bandpass circuit was used to generate the low-pass variables in the bandpass function (see section entitled “Data Processing and Calculations”). Representative flow cytometry histograms shows GFP expression for the data described herein (data not shown).

### Example 12: Higher-Order Signal-Processing Circuits for Converting a Single Analog Input into Multiple Distinct Outputs

Higher-order signal-processing circuits can be designed to convert a single analog input into multiple distinct outputs. For instance, analog-to-digital converters that convert input H₂O₂ into the expression of multiple genes were built (multi-bit analog-to-digital converters). For example, a circuit that can be used to encode ternary (three-valued) signals (logic gene circuits) was built. The circuit measures input H₂O₂ concentration and converts it into three gene expression states that represent a confirmed low concentration (“−1”), an intermediate concentration (“0”), or a confirmed high concentration (“+1”). To construct this circuit, the bandpass circuit above such that gfp was initially expressed by the proD promoter but would be shut off by Bxb1 production. A copy of rfp that could be activated by inversion of the promoter by PhiC1 production was then added. The “−1” state was defined as when >90% of cells were GFP positive and the “1” state as when >90% of cells were RFP positive. This resulted in three distinct gene expression states within the cells that were toggled at different H₂O₂ concentrations by using a pair of output signals that encode the information of a ternary output. In future work, the rfp and gfp outputs could be replaced by other genetic regulators that feed into downstream computing circuits. These types of circuits could be extended to implement ternary logic, to report inequalities (i.e. as <, =, >), or to encode distinct outputs at low or high input levels to actuate downstream circuits.

A circuit was also built where multiple comparators with different thresholds were each used to drive expression of a different fluorophore, thus implementing an ADC. This circuit classified H₂O₂ concentrations into one of four gene expression states in each cell ([gfp, rfp, bfp]=000, 100, 110, 111) due to successive Bxb1, PhiC31, and TP901 expression with increasing H₂O₂, thereby encoding 2 bits of information. The relative input ranges of the threshold circuits were 7.79, 5.08, and 6.42 for gfp, rfp, and bfp expression respectively, demonstrating that the ADC operates similarly in each concentration range. The resolution of an electronic analog-to-digital converter is a measure of the number of output discrete values encoded across a continuous input voltage range. An analogous figure of merit for genetic analog-to-digital converters was created, where the number of bits encoded across the ADC relative input range was measure (see section entitled “Data Processing and Calculations”). This relative resolution (RQ) was calculated for the ADC to be 3.84. Adding downstream XNOR and AND circuits to this ADC should implement a canonical 2-bit ADC that generates a binary 2-bit output.

Thus, examples of ternary (three-state) logic gene circuits are provided herein. OxyR is constitutively expressed and activates transcription of bxb1 and phiC31 in response to increasing concentrations of H₂O₂. Bxb1 unpairs the gfp cassette from the proD promoter, and PhiC31 unpairs the proD promoter from the gfp cassette and pairs it with the rfp cassette. The percent of cells expressing GFP and the percent of cells expressing RFP were fit to sigmoidal functions. The “−1” state is defined as >90% cells being GFP positive. The “+1” is defined as >90% of cells being RFP positive. The “0” state is when neither −1 or +1 conditions are met. Abstraction of ternary logic genetic circuit shows that H₂O₂ activates OxyR, which then activates expression of bxb1 and phiC31 depending upon the thresholds set by the promoters and RBS of their respective circuits. GFP expression is repressed by bxb1 OR phiC31 activation, whereas RFP activation is dependent upon phiC31 activation. For an example 2-bit analog-to-digital converter, OxyR is constitutively produced and activates transcription of bxb1, phiC31, and tp901 in response to increasing thresholds of H₂O₂. Bxb1, PhiC31, and TP901 invert gfp, rfp, and bfp, respectively, to enable expression from three different upstream proD promoters. Cells expressing GFP, RFP, or BFP were fit to sigmoidal functions. The transition band for each circuit is demarcated by a horizontal dashed line of the same color. Each transfer function had a similar relative input range. Abstraction of 2-bit analog-to-digital converter shows that H₂O₂ activates OxyR, which then activates expression of bxb1, phiC31, tp901 depending upon the thresholds set by the promoters and RBS of their respective circuits. Bxb1, PhiC31, and TP901 then activate gfp, rfp, and bfp expression, respectively. The errors (standard deviation) are derived from flow cytometry experiments of three biological replicates, each of which involved n>30,000 gated events. Representative flow cytometry histograms show GFP and RFP expression for the ternary logic circuits described in this example (data now shown). Representative flow cytometry histograms also show GFP, RFP and BFP expression from the analog-to-digital converter circuit described in this example (data now shown). Cells containing the 2-bit ADC were grown within flasks with different concentrations of H₂O₂ at a volume of 20 mL, which is a 100× greater volume than which was used to generate the data above. The data is the mean and standard deviation of the percent of fluorophore-positive cells from flow cytometry experiments with three biological replicates (data not shown).

### Example 13: Production of Mixed-Signal Processing Circuits

Analog-to-digital circuits can be further interfaced with digital circuits to form mixed-signal processing circuits. A variant of the bandpass circuit was built where the low-threshold comparator and medium-threshold comparator circuits both flip the directionality of gfp. This resulted in an analog-to-digital circuit where only intermediate H₂O₂ levels enable GFP production, which is analogous to an XOR gate on H₂O₂ concentrations digitized using two different thresholds. In addition, tp901 was placed under control of the TetR-repressed pLtetO promoter and constitutively expressed tetR, thereby making tp901 digitally inducible by anhydrotetracycline (aTc). tp901 was then used to control the direction of the promoter driving transcription of gfp. GFP levels were assayed at different H₂O₂ concentrations in the presence and absence of aTc and a majority of GFP-positive cells was found only at intermediate concentrations of H₂O₂ and when aTc was absent, thus implementing the concentration-dependent logic. Concentration-dependent logic allowed cells to carry out distinct activities at intermediate input levels, as opposed to extreme ones, and to encode a greater density of information into biological signals.

Thus, examples of mixed-signal computation and concentration-dependent logic are also provided. For a mixed-signal gene circuit, OxyR is constitutively produced and activates transcription of bxb1 and phiC31 at two different thresholds of H₂O₂. Both Bxb1 and PhiC31 can invert a gfp expression cassette. Bxb1-based flipping occurs at a lower H₂O₂ concentration than PhiC31-based flipping such that gfp is only in an upright orientation over an intermediate range of H₂O₂. Furthermore, TetR is constitutively produced and represses the pLtetO promoter; this repression is relieved by the presence of aTc. TP901 is expressed from the pLtetO promoter and inverts the proD promoter such that it cannot drive expression from an upright gfp cassette. The resulting circuit implements concentration-dependent logic with an output (GFP) that is ON only if an intermediate level of the input H₂O₂ is present and aTc is not present. Cells express GFP at different concentrations of H₂O₂ in the presence and absence of aTc. When aTc is absent, the circuit implements a bandpass response to H₂O₂, where the data is well-fit by the same transfer function as above, R²=0.94. When aTc is present, the circuit is OFF.

An abstraction of the mixed-signal gene circuit shows that H₂O₂ activates OxyR, which then activates expression of bxb1 and phiC31 depending upon the thresholds set by the promoters and RBS of their respective circuits. aTc activates expression of tp901 via inactivation of TetR. GFP is expressed when either Bxb1 or PhiC31 are present AND NOT when TP901 is activated. The errors (standard deviation) are derived from flow cytometry experiments of three biological replicates, each of which involved n>30,000 gated events.

### Example 14

Analog-to-digital converters (ADCs) are the complement of digital-to-analog converters (DACs): ADCs convert an analog input signal into discrete output signals, whereas DACs convert discrete input signals into analog output signals. For example, DACs implemented in living cells accepted two digital inputs and produced four different gene expression levels as outputs depending on the specific combination of inputs. Provided herein are ADCs that translate a single analog input in the form of inducer concentration to multiple discrete outputs, represented by triggering the expression of different genes.

Thus, examples of digital-to-analog converters and analog-to-digital converters, which are complementary systems that translate digital signals to analog signals, and vice versa, are also provided. In the digital computation paradigm, signals are defined as OFF or ON and computing is based on Boolean logic. In the analog computation paradigm, circuits convert continuous, analog inputs to continuous outputs according to mathematical relationships. Analog information is converted to digital information with analog-to-digital converters (ADC). Digital information is converted to analog information with digital-to-analog converters (DAC). A digital-to-analog converter that accepts various digital combinations of inputs and outputs quantized levels of a single output is also exemplified. An analog-to-digital converter that accepts the continuous, analog concentration of an input and classifies discrete ranges of the input to different output molecules is also exemplified. An analog-to-digital converter that accepts the continuous, analog concentration of an input and classifies discrete ranges of the input to discrete levels of a single output is also exemplified.

### Strains and Plasmids.

All plasmids were constructed with standard cloning procedures. Escherichia coli EPI300 (F⁻ mcrA Δ(mrr-hsdRMS-mcrBC) Φ80dlacZΔM15 ΔlacX74 recA1 endA1 araD139 Δ(ara, leu)7697 galU galK λ⁻ rpsL (Str^{R}) nupG trfA dhfr) was used for all experiments.

### Circuit Characterization.

Plasmids were transformed into chemically competent E. coli EPI300, plated on LB medium with appropriate antibiotics and grown overnight at 37° C. Antibiotic concentrations were Carbenicillin (50 μg/ml), Kanamycin (30 μg/ml), and Chloramphenicol (25 μg/ml). The next day, single colonies were inoculated into Teknova Hi-Def Azure Media with appropriate antibiotics and 0.2% glucose and incubated shaking aerobically at 37° C. for 16-18 hours. Cultures were then diluted 2500× into fresh media with Hi-Def Azure Media with appropriate antibiotics and 0.2% glucose and shaken for 20 minutes aerobically at 37° C. After 20 minutes, 200 μl of culture was transferred to a 96-well plate and H₂O₂ (Sigma Aldrich H1009-100 mL) was added at appropriate concentration via serial dilution. For the experiment in Example 13, aTc (anhydrotetracycline, Cayman Chemical 10009542) was added to a final concentration of 75 ng/ml. Plates were incubated aerobically with shaking at 30° C. for 20 hours for all experiments except those in FIGS. 10A-10C, in which plates were incubated for 3 hours. After incubation, the optical densities of cultures were measured at 600 nm in a plate reader. For experiments in FIGS. 12A-12G, FIGS. 13A-13C and FIGS. 14A-14E, cells were then assayed on the flow cytometer. For all other experiments, cells were washed with PBS, diluted 8× into fresh Hi-Def Azure Media with appropriate antibiotics, 0.4% glycerol, and 1× CopyControl Induction Solution (Epicentre), and incubated, shaking aerobically for a further 10 hours at 30° C. After this incubation, the optical densities of cultures were measured at 600 nm in a plate reader. For all flow cytometer experiments, cells were diluted into ice-cold 1×PBS to an optical density at 600 nm of less than 0.02 and assayed on a BD LSRFortessa using the high-throughput sampler. At least 30,000 gated events were recorded. GFP expression was measured via the FITC channel, RFP expression was measured via the TexasRed channel, and BFP expression was measured via the Pacific Blue channel. FCS files were exported and processed in FlowJo software. Events were gated for live E. coli via forward scatter area and side scatter area and then analyzed as in Supplementary Information Section 1. At least three biological replicates were conducted for each experiment.

Mixed-signal processing enables a wide range of industrial, diagnostic, and therapeutic engineered cell applications. For example, cells may be designed to produce quorum-sensing signals that trigger multiple distinct production pathways as the quorum-sensing molecules accumulate in a bioreactor. The first phase may be focused on biomass accumulation, the second phase dedicated to secreting the desired product, such as a biologic protein drug fused to a secretion tag, and the third committed to secreting product-modifying enzymes, such as a protease to separate the secretion tag from the active drug. Such behavior may be programmed with an ADC that senses the concentration of an accumulating quorum-sensing molecule as an input and triggers successive circuits with higher concentrations. Towards such industrial applications, the operational-volume of the ADC circuit was scaled up by 100× and the circuit functioned, albeit with shifted thresholds.

In addition, cells may be designed to detect continuous quantities of multiple biomarkers, integrate these signals to diagnose disease conditions, and produce reporter output(s) for non-invasive biosensing applications. Reporting on disease states and severity with digitized outputs (e.g., different fluorescent or colorimetric reporters), in some instance, may be more robust than analog outputs (e.g., a single fluorescent reporter expressed at different levels) since the latter is more susceptible to noise. Analog-to-digital converters may also be used as peak detectors due to the inherent memory feature of recombinase-based switches. For example, probiotic bacteria may be engineered to remember the maximum concentration of a biomarker that they detected while passing through the intestine. Similar circuits may be used to create environmental sensors that sense and record maximum pollutant levels.

Mixed-signal circuits may also be useful for engineering cell therapies whose therapeutic outputs are regulated by quantitative levels of disease biomarkers. For example, mammalian gene circuits may be designed such that blood glucose levels below the normal region (“−1” in a ternary logic system) switch on glucagon secretion, blood glucose levels in the desired region (“0” in a ternary logic system) result in no hormone secretion, and blood glucose levels above the normal region (“1” in in a ternary logic system) trigger insulin secretion. The ability to trigger distinct outputs in response to different conditions enables “homeostatic” therapies. Such applications benefit from resettable mixed-signal circuits, which may be implemented using transcriptional regulators, rather than the permanent-memory mixed-signal circuits described here.

In summary, mixed-signal gene circuits merge analog and digital signal processing to enable both continuous information sensing and robust multi-signal integration and computing in living cells. This hybrid analog-digital computational paradigm allows synthetic biological systems to begin to approach the nuanced complexities found in natural biological systems.

TABLE 1
Plasmids and Parts

| FIG. | Plasmids |
|---|---|
| 15A | pZS2oxySp*-RBS30-Bxbi-proD-oxyR + Bxbi GFP BAC Reporter |
| | pZS2katGp-RBS31-PhiC31-proD-oxyR + PhiC31 GFP BAC |
| | Reporter |
| | pZS2katGp-RBS33-TP901-proD-oxyR + TP901 GFP BAC |
| | Reporter |
| | pZS2oxySp*-RBS30-Bxbi-proD-oxyR + pZS1katGp-RBS31- |
| | PhiC31- proD-oxyR + Bxbi + PhiC31 GFP Bandpass BAC |
| | Reporter |
| | pZS2oxySp*-RBS30-Bxbi-proD-oxyR + pZS1katGp-RBS33- |
| | TP901- proD-oxyR + Bxbi + TP901 GFP Bandpass BAC |
| | Reporter |
| | pZS2oxySp*-RBS30-Bxbi-proD-oxyR + pZS1katGp-RBS31- |
| | PhiC31-proD-oxyR + Ternary BAC Reporter |
| | pZSloxySp*-RBS30-bxbi-katGp-RBS31-PhiC31-proD-oxyR + |
| | pZS2katGp-RBS33-TP901-proD-oxyR + ADC BAC Reporter |
| | pZS2oxySp*-RBS30-Bxbi-proD-oxyR + pZS1(pLtetO-TP901-aav- |
| | proA-tetR)-katGp-PhiC31-proD-oxyR + Mixed-signal integration |
| | BAC Reporter |
| | pZS2oxySp-GFP-proD-oxyR |
| | pZS2oxySp*-RBS30-Bxbi-proD-oxyR + Bxbi GFP MCP Reporter |
| | pZS2oxySp*-RBS30-Bxbi-proD-oxyR + pBAC Bxbi TetR + |
| | pZA1pLtetO-GFP |
| | pZS2oxySp*-RBS30-Bxbi-proD-oxyR + pZS1katGp-RBS31- |
| | PhiC31-proD-oxyR + Bxbi GFP BAC Reporter |
| | pZS2oxySp*-RBS30-Bxbi-proD-oxyR + pZS1katGp-RBS31- |
| | PhiC31-proD-oxyR + PhiC31 GFP BAC Reporter |
| | pZS2oxySp*-RBS30-Bxbi-proD-oxyR + pZS1katGp-RBS33- |
| | TP901-proD-oxyR + Bxbi GFP BAC Reporter |
| | pZS2oxySp*-RBS30-Bxbi-proD-oxyR + pZS1katGp-RBS33- |
| | TP901-proD-oxyR + TP901 GFP BAC Reporter |

TABLE 2
List of Synthetic Parts

| Part Name | Description and Source |
|---|---|
| oxySp | Promoter for E. coli oxySp RNA (The EcoCyc Database) |
| katGp | Promoter for E. coli katG (The EcoCyc Database) |
| ahpCp | Promoter for E. coli ahpC (The EcoCyc Database) |
| proD | Strong constitutive promoter (Davis J. et al. Nucleic Acids Research 39, |
| | 1131-1141 (2010)) |
| proA | Weak constitutive promoter (Davis J. et al. Nucleic Acids Research 39, |
| | 1131-1141 (2010)) |
| pLtetO | tetR-regulated lambda phage promoter (Lutz R Nucleic Acids Research 25, |
| | 1203-1210 (1997)) |
| RBS30 | Ribosome binding site. BBa_B0030 (Registry of Standard Biological |
| | Parts) |
| RBS29 | Ribosome binding site. BBa_B0029 (Registry of Standard Biological |
| | Parts) |
| RBS33 | Ribosome binding site. BBa_B0033 (Registry of Standard Biological |
| | Parts) |
| RBS31 | Ribosome binding site. BBa_B0031 (Registry of Standard Biological |
| | Parts) |
| “RBS” with no | RBS with maximized strength using computational method |
| number | |
| RiboJ | Ribozyme-insulator |
| oxyR | oxyR protein-coding sequence |
| mCherry | mCherry fluorescent protein coding sequence. BBa_J06504 (Registry of |
| | Standard Biological Parts) |
| mKate | mKate fluorescent protein coding sequence (Shcherbo, D. et al. Biochem. |
| | J. 418, 567 (2009)) |
| azurite | Azurite fluorescent protein coding sequence (Mena, M. et al. Nat |
| | Biotechnol 24, 1569-1571 (2006)) |
| gfp | Gfpmut3 fluorescent protein coding sequence. BBa_K863120 (Registry of |
| | Standard Biological Parts) |
| Bxb1 | Bxb1 serine integrase protein coding sequence |
| phiC31 | PhiC31 serine integrase protein coding sequence |
| tp901 | TP901 serine integrase protein coding sequence |
| Bxb1B/P | Bxb1 AttB and Bxbi AttP DNA recombination sites |
| PhiCB/P | PhiC31 AttB and Bxbi AttP DNA recombination sites |
| TP901B/P | TP901 AttB and Bxbi AttP DNA recombination sites |
| ECK120029600 | Synthetic transcriptional terminator (Chen, Y. et al. Nat Meth 10, 659-664 |
| | (2013)) |
| ECK120033737 | Synthetic transcriptional terminator (Chen, Y. et al. Nat Meth 10, 659-664 |
| | (2013)) |
| AAV | AAV degradation tag |
| TermT1 | Transcriptional Terminator T (Lutz R Nucleic Acids Research 25, 1203-1210 |
| | (1997)) |
| TermT0 | Transcriptional Terminator T0 (Lutz R Nucleic Acids Research 25, 1203-1210 |
| | (1997)) |
| p15A | Medium-copy number plasmid origin of replication (Lutz R Nucleic Acids |
| | Research 25, 1203-1210 (1997)) |
| pSC101 | Low-copy number plasmid origin of replication (Lutz R Nucleic Acids |
| | Research 25, 1203-1210 (1997)) |
| ampR | Ampicillin-resistance cassette (Lutz R Nucleic Acids Research 25, 1203-1210 |
| | (1997)) |
| kanR | Kanamycin-resistance cassette (Lutz R Nucleic Acids Research 25, 1203-1210 |
| | (1997)) |
| cmR | Spectinomycin-resistance cassette (Lutz R Nucleic Acids Research 25, |
| | 1203-1210 (1997)) |
| oriV | Trfa-activated plasmid origin of replication |
| BAC/F/RepE | Bacterial artificial chromosome replication factors and origin |
| incW | |
| parA/B/C | |

TABLE 3
DNA sequence of synthetic parts

| | | SEQ ID |
|---|---|---|
| Part Name | Description and Source | NO: |
| oxySp | TTCATTATCCATCCTCCATCGCCACGATAGTTCATGGCGATAGG | 1 |
| | TAGAATAGCAATGAACGATTATCCCTATCAAGCATTCTGACTGA | |
| | GCATTGCTCACA | |
| katGp | TGTGGCTTTTATGAAAATCACACAGTGATCACAAATTTTAAACA | 2 |
| | GAGCACAAAATGCTGCCTCGAAATGAGGGCGGGAAAATAAGGT | |
| | TATCAGCCTTGTTTTCTCCCTCATTACTTGAAGGATATGAAGCTA | |
| | AAACCCTTTTTTATAAAGCATTTGTCCGAATTCGGACATAATCA | |
| | AAAAAGCTTAATTAAGATCAATTTGATCTACATCTCTTTAACCA | |
| | ACAATATGTAAGATCTCAACTATCGCATCCGTGGATTAATTCAA | |
| | TTATAACTTCTCTCTAACGCTGTGTATCGTAACGGTAACACTGT | |
| | AGAGGGGAGCACATTGATG | |
| ahpCp | GCTTAGATCAGGTGATTGCCCTTTGTTTATGAGGGTGTTGTAAT | 3 |
| | CCATGTCGTTGTTGCATTTGTAAGGGCAACACCTCAGCCTGCAG | |
| | GCAGGCACTGAAGATACCAAAGGGTAGTTCAGATTACACGGTC | |
| | ACCTGGAAAGGGGGCCATTTTACTTTTTATCGCCGCTGGCGGTG | |
| | CAAAGTTCACAAAGTTGTCTTACGAAGGTTGTAAGGTAAAACTT | |
| | ATCGATTTGATAATGGAAACGCATTAGCCGAATCGGCAAAAAT | |
| | TGGTTACCTTACATCTCATCGAAAACACGGAGGAAGTATAGATG | |
| proD | CACAGCTAACACCACGTCGTCCCTATCTGCTGCCCTAGGTCTAT | 4 |
| | GAGTGGTTGCTGGATAACTTTACGGGCATGCATAAGGCTCGTAT | |
| | AATATATTCAGGGAGACCACAACGGTTTCCCTCTACAAATAATT | |
| | TTGTTTAACTTT | |
| proA | CACAGCTAACACCACGTCGTCCCTATCTGCTGCCCTAGGTCTAT | 5 |
| | GAGTGGTTGCTGGATAACTTTACGGGCATGCATAAGGCTCGTAG | |
| | GCTATATTCAGGGAGACCACAACGGTTTCCCTCTACAAATAATT | |
| | TTGTTTAACTTT | |
| pLtetO | TCCCTATCAGTGATAGAGATTGACATCCCTATCAGTGATAGAGA | 6 |
| | TACTGAGCACATCAGCAGGACGCACTGACC | |
| RBS30 | ATTAAAGAGGAGAAA | 7 |
| RBS29 | GGTTTCCTGTGTGAA | 8 |
| RBS33 | TCACACAGGAC | 9 |
| RBS31 | TCACACAGGAAACC | 10 |
| RBS for GFP | TAGTTTACGAGGTAAGGAGGTTTAAG | 11 |
| reporters | | |
| RBS for mCherry | ACTAACAATAAAGTATAAGGAGGTCTACA | 12 |
| reporters | | |
| RBS for mKate | ACGACATAAAATTAATAAGGAGGTAAAA | 13 |
| reporters | | |
| RBS for Azurite | TGAGTACGTAGGGAGGAGGTTAAAA | 14 |
| reporters | | |
| RiboJ | TTAAACAAAATTATTTGTAGAGGCTGTTTCGTCCTCACGGACTC | 15 |
| | ATCAGACCGGAAAGCACATCCGGTGACAGCT | |
| oxyR | ATGAATATTCGTGATCTTGAGTACCTGGTGGCATTGGCTGAACA | 16 |
| | CCGCCATTTTCGGCGTGCGGCAGATTCCTGCCACGTTAGCCAGC | |
| | GACGCTTAGCGGGCAAATTCGTAAGCTGGAAGATGAGCTGGG | |
| | CGTGATGTTGCTGGAGCGGACCAGCCGTAAAGTGTTGTTCACCC | |
| | AGGCGGGAATGCTGCTGGTGGATCAGGCGCGTACCGTGCTGCG | |
| | TGAGGTGAAAGTCCTTAAAGAGATGGCAAGCCAGCAGGGCGAG | |
| | ACGATGTCCGGACCGCTGCACATTGGTTTGATTCCCACAGTTGG | |
| | ACCGTACCTGCTACCGCATATTATCCCTATGCTGCACCAGACCT | |
| | TTCCAAAGCTGGAAATGTATCTGCATGAAGCACAGACCCACCA | |
| | GTTACTGGCGCAACTGGACAGCGGCAAACTCGATTGCGTGATCC | |
| | TCGCGCTGGTGAAAGAGAGCGAAGCATTCATTGAAGTGCCGTT | |
| | GTTTGATGAGCCAATGTTGCTGGCTATCTATGAAGATCACCCGT | |
| | GGGCGAACCGCGAATGCGTACCGATGGCCGATCTGGCAGGGGA | |
| | AAAACTGCTGATGCTGGAAGATGGTCACTGTTTGCGCGATCAGG | |
| | CAATGGGTTTCTGTTTTGAAGCCGGGGCGGATGAAGATACACAC | |
| | TTCCGCGCGACCAGCCTGGAAACTCTGCGCAACATGGTGGCGG | |
| | CAGGTAGCGGGATCACTTTACTGCCAGCGCTGGCTGTGCCGCCG | |
| | GAGCGCAAACGCGATGGGGTTGTTTATCTGCCGTGCATTAAGCC | |
| | GGAACCACGCCGCACTATTGGCCTGGTTTATCGTCCTGGCTCAC | |
| | CGCTGCGCAGCCGCTATGAGCAGCTGGCAGAGGCCATCCGCGC | |
| | AAGAATGGATGGCCATTTCGATAAAGTTTTAAAACAGGCGGTTT | |
| | AA | |
| mCherry | ATGGTGAGCAAGGGCGAAGAAGATAACATGGCCATCATCAAGG | 17 |
| | AGTTCATGCGCTTCAAGGTGCACATGGAGGGCTCCGTGAACGG | |
| | CCACGAGTTCGAGATCGAGGGCGAGGGCGAGGGCCGCCCCTAC | |
| | GAGGGCACCCAGACCGCCAAGCTGAAGGTGACCAAGGGTGGCC | |
| | CCCTGCCCTTCGCCTGGGACATCCTGTCCCCTCAGTTCATGTACG | |
| | GCTCCAAGGCCTACGTGAAGCACCCCGCCGACATCCCCGACTAC | |
| | TTGAAGCTGTCCTTCCCCGAGGGCTTCAAGTGGGAGCGCGTGAT | |
| | GAACTTCGAGGACGGCGGCGTGGTGACCGTGACCCAGGACTCC | |
| | TCCCTGCAGGACGGCGAGTTCATCTACAAGGTGAAGCTGCGCG | |
| | GCACCAACTTCCCCTCCGACGGCCCCGTAATGCAGAAGAAGAC | |
| | CATGGGCTGGGAGGCCTCCTCCGAGCGGATGTACCCCGAGGAC | |
| | GGCGCCCTGAAGGGCGAGATCAAGCAGAGGCTGAAGCTGAAGG | |
| | ACGGCGGCCACTACGACGCTGAGGTCAAGACCACCTACAAGGC | |
| | CAAGAAGCCCGTGCAGCTGCCCGGCGCCTACAACGTCAACATC | |
| | AAGTTGGACATCACCTCCCACAACGAGGACTACACCATCGTGG | |
| | AACAGTACGAACGCGCCGAGGGCCGCCACTCCACCGGCGGCAT | |
| | GGACGAGCTGTACAAGTAA | |
| mKate | ATGGTTTCCAAAGGTGAAGAACTGATTAAAGAAAACATGCACA | 18 |
| | TGAAGCTGTACATGGAAGGTACTGTAAACAACCACCACTTCAA | |
| | ATGTACCAGCGAAGGCGAAGGCAAACCGTATGAGGGCACCCAA | |
| | ACCATGCGTATCAAAGTTGTGGAAGGCGGTCCGCTGCCGTTTGC | |
| | ATTCGACATCCTGGCGACCAGCTTCATGTACGGCAGCAAAACCT | |
| | TCATCAACCACACTCAAGGTATCCCGGATTTTTTCAAACAGAGC | |
| | TTCCCGGAGGGCTTTACCTGGGAACGCGTTACGACGTATGAAGA | |
| | TGGTGGCGTCCTGACCGCTACGCAGGACACGTCTCTGCAGGATG | |
| | GCTGTCTGATCTATAACGTTAAAATTCGTGGTGTTAATTTCCCG | |
| | AGCAACGGCCCGGTTATGCAGAAAAAAACGCTGGGCTGGGAAG | |
| | CATCCACCGAAATGCTGTACCCGGCTGACGGCGGCCTGGAAGG | |
| | CCGTTCTGATATGGCGCTGAAACTGGTTGGTGGCGGCCACCTGA | |
| | TCTGTAACCTGAAAACTACTTACCGCAGCAAAAAACCGGCTAA | |
| | AAACCTGAAAATGCCGGGCGTATATTATGTCGACCGCCGTCTGG | |
| | AACGTATCAAAGAAGCGGACAAAGAAACCTATGTCGAACAGCA | |
| | TGAAGTGGCAGTGGCACGCTATTGCGATCTGCCTTCCAAACTGG | |
| | GCCACAAACTGAACTAA | |
| azurite | ATGTCTAAAGGTGAAGAATTATTCACTGGTGTTGTCCCAATTTT | 19 |
| | GGTTGAATTAGATGGTGATGTTAATGGTCACAAATTTTCTGTCT | |
| | CCGGTGAAGGTGAAGGTGATGCTACGTACGGTAAATTGACCTT | |
| | AAAATTTATTTGTACTACTGGTAAATTGCCAGTTCCATGGCCAA | |
| | CCTTAGTAACTACTTTGAGCCATGGTGTTCAATGTTTTTCTAGAT | |
| | ACCCAGATCATATGAAACAACATGACTTTTTCAAGTCTGCCATG | |
| | CCAGAAGGTTATGTTCAAGAAAGAACTATTTTTTTCAAAGATGA | |
| | CGGTAACTACAAGACCAGAGCTGAAGTCAAGTTTGAAGGTGAT | |
| | ACCTTAGTTAATAGAATCGAATTAAAAGGTATTGATTTTAAAGA | |
| | AGATGGTAACATTTTAGGTCACAAATTGGAATACAACTTCAACT | |
| | CTCACAATATATACATCATGGCTGACAAACAAAAGAATGGTAT | |
| | CAAAGTGAACTTCAAAATTAGACACAACATTGAAGATGGTTCT | |
| | GTTCAATTAGCTGACCATTATCAACAAAATACTCCAATTGGTGA | |
| | TGGTCCAGTCTTGTTACCAGACAACCATTACTTATCCACCCAAT | |
| | CAGCCTTATCCAAAGATCCAAACGAAAAGAGAGACCACATGGT | |
| | CCTGTTAGAATTTAGGACTGCTGCTGGTATTACCCATGGTATGG | |
| | ATGAATTGTACAAATAA | |
| gfp | ATGAGTAAAGGAGAAGAACTTTTCACTGGAGTTGTCCCAATTCT | 20 |
| | TGTTGAATTAGATGGTGATGTTAATGGGCACAAATTTTCTGTCA | |
| | GTGGAGAGGGTGAAGGTGATGCAACATACGGAAAACTTACCCT | |
| | TAAATTTATTTGCACTACTGGAAAACTACCTGTTCCATGGCCAA | |
| | CACTTGTCACTACTTTCGGTTATGGTGTTCAATGCTTTGCGAGAT | |
| | ACCCAGATCATATGAAACAGCATGACTTTTTCAAGAGTGCCATG | |
| | CCCGAAGGTTATGTACAGGAAAGAACTATATTTTTCAAAGATGA | |
| | CGGGAACTACAAGACACGTGCTGAAGTCAAGTTTGAAGGTGAT | |
| | ACCCTTGTTAATAGAATCGAGTTAAAAGGTATTGATTTTAAAGA | |
| | AGATGGAAACATTCTTGGACACAAATTGGAATACAACTATAAC | |
| | TCACACAATGTATACATCATGGCAGACAAACAAAAGAATGGAA | |
| | TCAAAGTTAACTTCAAAATTAGACACAACATTGAAGATGGAAG | |
| | CGTTCAACTAGCAGACCATTATCAACAAAATACTCCAATTGGCG | |
| | ATGGCCCTGTCCTTTTACCAGACAACCATTACCTGTCCACACAA | |
| | TCTGCCCTTTCGAAAGATCCCAACGAAAAGAGAGACCACATGG | |
| | TCCTTCTTGAGTTTGTAACAGCTGCTGGGATTACACATGGCATG | |
| | GATGATCTCTACAAATAA | |
| Bxb1 | ATGAGAGCCCTGGTAGTCATCCGCCTGTCCCGCGTCACCGATGC | 21 |
| | TACGACTTCACCGGAGCGTCAGCTGGAGTCTTGCCAGCAGCTCT | |
| | GCGCCCAGCGCGGCTGGGACGTCGTCGGGGTAGCGGAGGATCT | |
| | GGACGTCTCCGGGGCGGTCGATCCGTTCGACCGGAAGCGCAGA | |
| | CCGAACCTGGCCCGGTGGCTAGCGTTCGAGGAGCAACCGTTCG | |
| | ACGTGATCGTGGCGTACCGGGTAGACCGGTTGACCCGATCGATC | |
| | CGGCATCTGCAGCAGCTGGTCCACTGGGCCGAGGACCACAAGA | |
| | AGCTGGTCGTCTCCGCGACCGAAGCGCACTTCGATACGACGAC | |
| | GCCGTTTGCGGCGGTCGTCATCGCGCTTATGGGAACGGTGGCGC | |
| | AGATGGAATTAGAAGCGATCAAAGAGCGGAACCGTTCGGCTGC | |
| | GCATTTCAATATCCGCGCCGGGAAATACCGAGGATCCCTGCCGC | |
| | CGTGGGGATACCTGCCTACGCGCGTGGACGGGGAGTGGCGGCT | |
| | GGTGCCGGACCCTGTGCAGCGAGAGCGCATCCTCGAGGTGTAT | |
| | CACCGCGTCGTCGACAACCACGAGCCGCTGCACCTGGTGGCCC | |
| | ACGACCTGAACCGGCGTGGTGTCCTGTCGCCGAAGGACTACTTC | |
| | GCGCAGCTGCAAGGCCGCGAGCCGCAGGGCCGGGAGTGGTCGG | |
| | CTACCGCGCTGAAGCGATCGATGATCTCCGAGGCGATGCTCGG | |
| | GTACGCGACTCTGAACGGTAAGACCGTCCGAGACGACGACGGA | |
| | GCCCCGCTGGTGCGGGCTGAGCCGATCCTGACCCGTGAGCAGCT | |
| | GGAGGCGCTGCGCGCCGAGCTCGTGAAGACCTCCCGGGCGAAG | |
| | CCCGCGGTGTCTACCCCGTCGCTGCTGCTGCGGGTGTTGTTCTGT | |
| | GCGGTGTGCGGGGAGCCCGCGTACAAGTTCGCCGGGGGAGGAC | |
| | GTAAGCACCCGCGCTACCGCTGCCGCTCGATGGGGTTCCCGAAG | |
| | CACTGCGGGAACGGCACGGTGGCGATGGCCGAGTGGGACGCGT | |
| | TCTGCGAGGAGCAGGTGCTGGATCTGCTCGGGGACGCGGAGCG | |
| | TCTGGAGAAAGTCTGGGTAGCCGGCTCGGACTCCGCGGTCGAA | |
| | CTCGCGGAGGTGAACGCGGAGCTGGTGGACCTGACGTCGCTGA | |
| | TCGGCTCCCCGGCCTACCGGGCCGGCTCTCCGCAGCGAGAAGC | |
| | ACTGGATGCCCGTATTGCGGCGCTGGCCGCGCGGCAAGAGGAG | |
| | CTGGAGGGTCTAGAGGCTCGCCCGTCTGGCTGGGAGTGGCGCG | |
| | AGACCGGGCAGCGGTTCGGGGACTGGTGGCGGGAGCAGGACAC | |
| | CGCGGCAAAGAACACCTGGCTTCGGTCGATGAACGTTCGGCTG | |
| | ACGTTCGACGTCCGCGGCGGGCTGACTCGCACGATCGACTTCGG | |
| | GGATCTGCAGGAGTACGAGCAGCATCTCAGGCTCGGCAGCGTG | |
| | GTCGAACGGCTACACACCGGGATGTCG | |
| phiC31 | ATGACACAAGGGGTTGTGACCGGGGTGGACACGTACGCGGGTG | 22 |
| | CTTACGACCGTCAGTCGCGCGAGCGCGAGAATTCGAGCGCAGC | |
| | AAGCCCAGCGACACAGCGTAGCGCCAACGAAGACAAGGCGGCC | |
| | GACCTTCAGCGCGAAGTCGAGCGCGACGGGGGCCGGTTCAGGT | |
| | TCGTCGGGCATTTCAGCGAAGCGCCGGGCACGTCGGCGTTCGG | |
| | GACGGCGGAGCGCCCGGAGTTCGAACGCATCCTGAACGAATGC | |
| | CGCGCCGGGCGGCTCAACATGATCATTGTCTATGACGTGTCGCG | |
| | CTTCTCGCGCCTGAAGGTCATGGACGCGATTCCGATTGTCTCGG | |
| | AATTGCTCGCCCTGGGCGTGACGATTGTTTCCACTCAGGAAGGC | |
| | GTCTTCCGGCAGGGAAACGTCATGGACCTGATTCACCTGATTAT | |
| | GCGGCTCGACGCGTCGCACAAAGAATCTTCGCTGAAGTCGGCG | |
| | AAGATTCTCGACACGAAGAACCTTCAGCGCGAATTGGGCGGGT | |
| | ACGTCGGCGGGAAGGCGCCTTACGGCTTCGAGCTTGTTTCGGAG | |
| | ACGAAGGAGATCACGCGCAACGGCCGAATGGTCAATGTCGTCA | |
| | TCAACAAGCTTGCGCACTCGACCACTCCCCTTACCGGACCCTTC | |
| | GAGTTCGAGCCCGACGTAATCCGGTGGTGGTGGCGTGAGATCA | |
| | AGACGCACAAACACCTTCCCTTCAAGCCGGGCAGTCAAGCCGC | |
| | CATTCACCCGGGCAGCATCACGGGGCTTTGTAAGCGCATGGAC | |
| | GCTGACGCCGTGCCGACCCGGGGCGAGACGATTGGGAAGAAGA | |
| | CCGCTTCAAGCGCCTGGGACCCGGCAACCGTTATGCGAATCCTT | |
| | CGGGACCCGCGTATTGCGGGCTTCGCCGCTGAGGTGATCTACAA | |
| | GAAGAAGCCGGACGGCACGCCGACCACGAAGATTGAGGGTTAC | |
| | CGCATTCAGCGCGACCCGATCACGCTCCGGCCGGTCGAGCTTGA | |
| | TTGCGGACCGATCATCGAGCCCGCTGAGTGGTATGAGCTTCAGG | |
| | CGTGGTTGGACGGCAGGGGGCGCGGCAAGGGGCTTTCCCGGGG | |
| | GCAAGCCATTCTGTCCGCCATGGACAAGCTGTACTGCGAGTGTG | |
| | GCGCCGTCATGACTTCGAAGCGCGGGGAAGAATCGATCAAGGA | |
| | CTCTTACCGCTGCCGTCGCCGGAAGGTGGTCGACCCGTCCGCAC | |
| | CTGGGCAGCACGAAGGCACGTGCAACGTCAGCATGGCGGCACT | |
| | CGACAAGTTCGTTGCGGAACGCATCTTCAACAAGATCAGGCAC | |
| | GCCGAAGGCGACGAAGAGACGTTGGCGCTTCTGTGGGAAGCCG | |
| | CCCGACGCTTCGGCAAGCTCACTGAGGCGCCTGAGAAGAGCGG | |
| | CGAACGGGCGAACCTTGTTGCGGAGCGCGCCGACGCCCTGAAC | |
| | GCCCTTGAAGAGCTGTACGAAGACCGCGCGGCAGGCGCGTACG | |
| | ACGGACCCGTTGGCAGGAAGCACTTCCGGAAGCAACAGGCAGC | |
| | GCTGACGCTCCGGCAGCAAGGGGCGGAAGAGCGGCTTGCCGAA | |
| | CTTGAAGCCGCCGAAGCCCCGAAGCTTCCCCTTGACCAATGGTT | |
| | CCCCGAAGACGCCGACGCTGACCCGACCGGCCCTAAGTCGTGG | |
| | TGGGGGCGCGCGTCAGTAGACGACAAGCGCGTGTTCGTCGGGC | |
| | TCTTCGTAGACAAGATCGTTGTCACGAAGTCGACTACGGGCAGG | |
| | GGGCAGGGAACGCCCATCGAGAAGCGCGCTTCGATCACGTGGG | |
| | CGAAGCCGCCGACCGACGACGACGAAGACGACGCCCAGGACG | |
| | GCACGGAAGACGTAGCGGCG | |
| tp901 | ATGACTAAGAAAGTAGCAATCTATACACGAGTATCCACTACTA | 23 |
| | ACCAAGCAGAGGAAGGCTTCTCAATTGATGAGCAAATTGACCG | |
| | TTTAACAAAATATGCTGAAGCAATGGGGTGGCAAGTATCTGAT | |
| | ACTTATACTGATGCTGGTTTTTCAGGGGCCAAACTTGAACGCCC | |
| | AGCAATGCAAAGATTAATCAACGATATCGAGAATAAAGCTTTT | |
| | GATACAGTTCTTGTATATAAGCTAGACCGCCTTTCACGTAGTGT | |
| | AAGAGATACTCTTTATCTTGTTAAGGATGTGTTCACAAAAAATA | |
| | AAATAGACTTTATCTCGCTTAATGAAAGTATTGATACTTCTTCTG | |
| | CTATGGGTAGCTTGTTTCTCACTATTCTTTCTGCAATTAATGAGT | |
| | TTGAAAGAGAGAATATAAAAGAACGCATGACTATGGGTAAACT | |
| | AGGGCGAGCGAAATCTGGTAAGTCTATGATGTGGACTAAGACA | |
| | GCTTTTGGGTATTACCACAACAGAAAGACAGGTATATTAGAAAT | |
| | TGTTCCTTTACAAGCTACAATAGTTGAACAAATATTCACTGATT | |
| | ATTTATCAGGAATATCACTTACAAAATTAAGAGATAAACTCAAT | |
| | GAATCTGGACACATCGGTAAAGATATACCGTGGTCTTATCGTAC | |
| | CCTAAGACAAACACTTGATAATCCAGTTTACTGTGGTTATATCA | |
| | AATTTAAGGACAGCCTATTTGAAGGTATGCACAAACCAATTATC | |
| | CCTTATGAGACTTATTTAAAAGTTCAAAAAGAGCTAGAAGAAA | |
| | GACAACAGCAGACTTATGAAAGAAATAACAACCCTAGACCTTT | |
| | CCAAGCTAAATATATGCTGTCAGGGATGGCAAGGTGCGGTTACT | |
| | GTGGAGCACCTTTAAAAATTGTTCTTGGCCACAAAAGAAAAGA | |
| | TGGAAGCCGCACTATGAAATATCACTGTGCAAATAGATTTCCTC | |
| | GAAAAACAAAAGGAATTACAGTATATAATGACAATAAAAAGTG | |
| | TGATTCAGGAACTTATGATTTAAGTAATTTAGAAAATACTGTTA | |
| | TTGACAACCTGATTGGATTTCAAGAAAATAATGACTCCTTATTG | |
| | AAAATTATCAATGGCAACAACCAACCTATTCTTGATACTTCGTC | |
| | ATTTAAAAAGCAAATTTCACAGATCGATAAAAAAATACAAAAG | |
| | AACTCTGATTTGTACCTAAATGATTTTATCACTATGGATGAGTT | |
| | GAAAGATCGTACTGATTCCCTTCAGGCTGAGAAAAAGCTGCTTA | |
| | AAGCTAAGATTAGCGAAAATAAATTTAATGACTCTACTGATGTT | |
| | TTTGAGTTAGTTAAAACTCAGTTGGGCTCAATTCCGATTAATGA | |
| | ACTATCATATGATAATAAAAAGAAAATCGTCAACAACCTTGTAT | |
| | CAAAGGTTGATGTTACTGCTGATAATGTAGATATCATATTTAAA | |
| | TTCCAACTCGCT | |
| Bxb1B | CGGCCGGCTTGTCGACGACGGCGGTCTCCGTCGTCAGGATCATC | 24 |
| | CGGGC | |
| Bxb1P | GTCGTGGTTTGTCTGGTCAACCACCGCGGTCTCAGTGGTGTACG | 25 |
| | GTACAAACCCCGAC | |
| PhiCB | TGCGGGTGCCAGGGCGTGCCCTTGGGCTCCCCGGGCGCGTACTC | 26 |
| | C | |
| PhiCP | GTGCCCCAACTGGGGTAACCTTTGAGTTCTCTCAGTTGGGGG | 27 |
| TP901B | ATGCCAACACAATTAACATCTCAATCAAGGTAAATGCTTTTTGC | 28 |
| | TTTTTTTGC | |
| TP901P | GCGAGTTTTTATTTCGTTTATTTCAATTAAGGTAACTAAAAAACT | 29 |
| | CCTTT | |
| ECK120029600 | TTCAGCCAAAAAACTTAAGACCGCCGGTCTTGTCCACTACCTTG | 30 |
| | CAGTAATGCGGTGGACAGGATCGGCGGTTTTCTTTTCTCTTCTC | |
| | AA | |
| ECK120033737 | GGAAACACAGAAAAAAGCCCGCACCTGACAGTGCGGGCTTTTT | 31 |
| | TTTTCGACCAAAGG | |
| AAV + stop codon | GCAGCAAACGACGAAAACTACGCTGCAGCAGTTTAG | 32 |
| TermT1 | GGCATCAAATAAAACGAAAGGCTCAGTCGAAAGACTGGGCCTT | 33 |
| | TCGTTTTATCTGTTGTTTGTCGGTGAACGCTCTCCTGAGTAGGAC | |
| | AAATCCGCCGCCCTAGA | |
| TermT0 | GCTTGGACTCCTGTTGATAGATCCAGTAATGACCTCAGAACTCC | 34 |
| | ATCTGGATTTGTTCAGAACGCTCGGTTGCCGCCGGGCGTTTTTT | |
| | ATTGGTGAGAATCCAAGC | |
| p15A | CGGTCGTTCGACTGCGGCGAGCGGAAATGGCTTACGAACGGGG | 35 |
| | CGGAGATTTCCTGGAAGATGCCAGGAAGATACTTAACAGGGAA | |
| | GTGAGAGGGCCGCGGCAAAGCCGTTTTTCCATAGGCTCCGCCCC | |
| | CCTGACAAGCATCACGAAATCTGACGCTCAAATCAGTGGTGGC | |
| | GAAACCCGACAGGACTATAAAGATACCAGGCGTTTCCCCCTGG | |
| | CGGCTCCCTCGTGCGCTCTCCTGTTCCTGCCTTTCGGTTTACCGG | |
| | TGTCATTCCGCTGTTATGGCCGCGTTTGTCTCATTCCACGCCTGA | |
| | CACTCAGTTCCGGGTAGGCAGTTCGCTCCAAGCTGGACTGTATG | |
| | CACGAACCCCCCGTTCAGTCCGACCGCTGCGCCTTATCCGGTAA | |
| | CTATCGTCTTGAGTCCAACCCGGAAAGACATGCAAAAGCACCA | |
| | CTGGCAGCAGCCACTGGTAATTGATTTAGAGGAGTTAGTCTTGA | |
| | AGTCATGCGCCGGTTAAGGCTAAACTGAAAGGACAAGTTTTGG | |
| | TGACTGCGCTCCTCCAAGCCAGTTACCTCGGTTCAAAGAGTTGG | |
| | TAGCTCAGAGAACCTTCGAAAAACCGCCCTGCAAGGCGGTTTTT | |
| | TCGTTTTCAGAGCAAGAGATTACGCGCAGACCAAAACGATCTC | |
| | AAGAAGATCATCTTATTAATCAGATAAAATATTTCTAGATTTCA | |
| | GTGCAATTTATCTCTTCAAATGTAGCACCTGAAGTCAGCCCCAT | |
| | ACGATATAAGTTGTT | |
| pSC101 | GTACGGGTTTTGCTGCCCGCAAACGGGCTGTTCTGGTGTTGCTA | 36 |
| | GTTTGTTATCAGAATCGCAGATCCGGCTTCAGGTTTGCCGGCTG | |
| | AAAGCGCTATTTCTTCCAGAATTGCCATGATTTTTTCCCCACGG | |
| | GAGGCGTCACTGGCTCCCGTGTTGTCGGCAGCTTTGATTCGATA | |
| | AGCAGCATCGCCTGTTTCAGGCTGTCTATGTGTGACTGTTGAGC | |
| | TGTAACAAGTTGTCTCAGGTGTTCAATTTCATGTTCTAGTTGCTT | |
| | TGTTTTACTGGTTTCACCTGTTCTATTAGGTGTTACATGCTGTTC | |
| | ATCTGTTACATTGTCGATCTGTTCATGGTGAACAGCTTTAAATG | |
| | CACCAAAAACTCGTAAAAGCTCTGATGTATCTATCTTTTTTACA | |
| | CCGTTTTCATCTGTGCATATGGACAGTTTTCCCTTTGATATCTAA | |
| | CGGTGAACAGTTGTTCTACTTTTGTTTGTTAGTCTTGATGCTTCA | |
| | CTGATAGATACAAGAGCCATAAGAACCTCAGATCCTTCCGTATT | |
| | TAGCCAGTATGTTCTCTAGTGTGGTTCGTTGTTTTTGCGTGAGCC | |
| | ATGAGAACGAACCATTGAGATCATGCTTACTTTGCATGTCACTC | |
| | AAAAATTTTGCCTCAAAACTGGTGAGCTGAATTTTTGCAGTTAA | |
| | AGCATCGTGTAGTGTTTTTCTTAGTCCGTTACGTAGGTAGGAAT | |
| | CTGATGTAATGGTTGTTGGTATTTTGTCACCATTCATTTTTATCT | |
| | GGTTGTTCTCAAGTTCGGTTACGAGATCCATTTGTCTATCTAGTT | |
| | CAACTTGGAAAATCAACGTATCAGTCGGGCGGCCTCGCTTATCA | |
| | ACCACCAATTTCATATTGCTGTAAGTGTTTAAATCTTTACTTATT | |
| | GGTTTCAAAACCCATTGGTTAAGCCTTTTAAACTCATGGTAGTT | |
| | ATTTTCAAGCATTAACATGAACTTAAATTCATCAAGGCTAATCT | |
| | CTATATTTGCCTTGTGAGTTTTCTTTTGTGTTAGTTCTTTTAATAA | |
| | CCACTCATAAATCCTCATAGAGTATTTGTTTTCAAAAGACTTAA | |
| | CATGTTCCAGATTATATTTTATGAATTTTTTTAACTGGAAAAGAT | |
| | AAGGCAATATCTCTTCACTAAAAACTAATTCTAATTTTTCGCTTG | |
| | AGAACTTGGCATAGTTTGTCCACTGGAAAATCTCAAAGCCTTTA | |
| | ACCAAAGGATTCCTGATTTCCACAGTTCTCGTCATCAGCTCTCT | |
| | GGTTGCTTTAGCTAATACACCATAAGCATTTTCCCTACTGATGTT | |
| | CATCATCTGAGCGTATTGGTTATAAGTGAACGATACCGTCCGTT | |
| | CTTTCCTTGTAGGGTTTTCAATCGTGGGGTTGAGTAGTGCCACA | |
| | CAGCATAAAATTAGCTTGGTTTCATGCTCCGTTAAGTCATAGCG | |
| | ACTAATCGCTAGTTCATTTGCTTTGAAAACAACTAATTCAGACA | |
| | TACATCTCAATTGGTCTAGGTGATTTTAATCACTATACCAATTG | |
| | AGATGGGCTAGTCAATGATAATTACTAGTCCTTTTCCTTTGAGTT | |
| | GTGGGTATCTGTAAATTCTGCTAGACCTTTGCTGGAAAACTTGT | |
| | AAATTCTGCTAGACCCTCTGTAAATTCCGCTAGACCTTTGTGTGT | |
| | TTTTTTTGTTTATATTCAAGTGGTTATAATTTATAGAATAAAGAA | |
| | AGAATAAAAAAAGATAAAAAGAATAGATCCCAGCCCTGTGTAT | |
| | AACTCACTACTTTAGTCAGTTCCGCAGTATTACAAAAGGATGTC | |
| | GCAAACGCTGTTTGCTCCTCTACAAAACAGACCTTAAAACCCTA | |
| | AAGGCTTAAGTAGCACCCTCGCAAGCTCGGGCAAATCGCTGAA | |
| | TATTCCTTTTGTCTCCGACCATCAGGCACCTGAGTCGCTGTCTTT | |
| | TTCGTGACATTCAGTTCGCTGCGCTCACGGCTCTGGCAGTGAAT | |
| | GGGGGTAAATGGCACTACAGGCGCCTTTTATGGATTCATGCAAG | |
| | GAAACTACCCATAATACAAGAAAAGCCCGTCACGGGCTTCTCA | |
| | GGGCGTTTTATGGCGGGTCTGCTATGTGGTGCTATCTGACTTTTT | |
| | GCTGTTCAGCAGTTCCTGCCCTCTGATTTTCCAGTCTGACCACTT | |
| | CGGATTATCCCGTGACAGGTCATTCAGACTGGCTAATGCACCCA | |
| | GTAAGGCAGCGGTATCATCAACAGGCTTACCCGTCTTACTGTCC | |
| | CTAGT | |
| ampR | AGGTGGCACTTTTCGGGGAAATGTGCGCGGAACCCCTATTTGTT | 37 |
| | TATTTTTCTAAATACATTCAAATATGTATCCGCTCATGAGACAA | |
| | TAACCCTGATAAATGCTTCAATAATATTGAAAAAGGAAGAGTA | |
| | TGAGTATTCAACATTTCCGTGTCGCCCTTATTCCCTTTTTTGCGG | |
| | CATTTTGCCTTCCTGTTTTTGCTCACCCAGAAACGCTGGTGAAA | |
| | GTAAAAGATGCTGAAGATCAGTTGGGTGCACGAGTGGGTTACA | |
| | TCGAACTGGATCTCAACAGCGGTAAGATCCTTGAGAGTTTTCGC | |
| | CCCGAAGAACGTTTTCCAATGATGAGCACTTTTAAAGTTCTGCT | |
| | ATGTGGCGCGGTATTATCCCGTATTGACGCCGGGCAAGAGCAA | |
| | CTCGGTCGCCGCATACACTATTCTCAGAATGACTTGGTTGAGTA | |
| | CTCACCAGTCACAGAAAAGCATCTTACGGATGGCATGACAGTA | |
| | AGAGAATTATGCAGTGCTGCCATAACCATGAGTGATAACACTG | |
| | CGGCCAACTTACTTCTGACAACGATCGGAGGACCGAAGGAGCT | |
| | AACCGCTTTTTTGCACAACATGGGGGATCATGTAACTCGCCTTG | |
| | ATCGTTGGGAACCGGAGCTGAATGAAGCCATACCAAACGACGA | |
| | GCGTGACACCACGATGCCTGTAGCAATGGCAACAACGTTGCGC | |
| | AAACTATTAACTGGCGAACTACTTACTCTAGCTTCCCGGCAACA | |
| | ATTAATAGACTGGATGGAGGCGGATAAAGTTGCAGGACCACTT | |
| | CTGCGCTCGGCCCTTCCGGCTGGCTGGTTTATTGCTGATAAATCT | |
| | GGAGCCGGTGAGCGTGGGTCTCGCGGTATCATTGCAGCACTGG | |
| | GGCCAGATGGTAAGCCCTCCCGTATCGTAGTTATCTACACGACG | |
| | GGGAGTCAGGCAACTATGGATGAACGAAATAGACAGATCGCTG | |
| | AGATAGGTGCCTCACTGATTAAGCATTGGTAACTGTCAGACCAA | |
| | GTTTAC | |
| kanR | TCGAACCCCAGAGTCCCGCTCAGAAGAACTCGTCAAGAAGGCG | 38 |
| | ATAGAAGGCGATGCGCTGCGAATCGGGAGCGGCGATACCGTAA | |
| | AGCACGAGGAAGCGGTCAGCCCATTCGCCGCCAAGCTCTTCAG | |
| | CAATATCACGGGTAGCCAACGCTATGTCCTGATAGCGGTCCGCC | |
| | ACACCCAGCCGGCCACAGTCGATGAATCCAGAAAAGCGGCCAT | |
| | TTTCCACCATGATATTCGGCAAGCAGGCATCGCCATGGGTCACG | |
| | ACGAGATCCTCGCCGTCGGGCATGCGCGCCTTGAGCCTGGCGA | |
| | ACAGTTCGGCTGGCGCGAGCCCCTGATGCTCTTCGTCCAGATCA | |
| | TCCTGATCGACAAGACCGGCTTCCATCCGAGTACGTGCTCGCTC | |
| | GATGCGATGTTTCGCTTGGTGGTCGAATGGGCAGGTAGCCGGAT | |
| | CAAGCGTATGCAGCCGCCGCATTGCATCAGCCATGATGGATACT | |
| | TTCTCGGCAGGAGCAAGGTGAGATGACAGGAGATCCTGCCCCG | |
| | GCACTTCGCCCAATAGCAGCCAGTCCCTTCCCGCTTCAGTGACA | |
| | ACGTCGAGCACAGCTGCGCAAGGAACGCCCGTCGTGGCCAGCC | |
| | ACGATAGCCGCGCTGCCTCGTCCTGCAGTTCATTCAGGGCACCG | |
| | GACAGGTCGGTCTTGACAAAAAGAACCGGGCGCCCCTGCGCTG | |
| | ACAGCCGGAACACGGCGGCATCAGAGCAGCCGATTGTCTGTTG | |
| | TGCCCAGTCATAGCCGAATAGCCTCTCCACCCAAGCGGCCGGA | |
| | GAACCTGCGTGCAATCCATCTTGTTCAATCATGCGAAACGATCC | |
| | TCATCCTGTCTCTTGATCAGATCTTGATCCCCTGCGCCATCAGAT | |
| | CCTTGGCGGCAAGAAAGCCATCCAGTTTACTTTGCAGGGCTTCC | |
| | CAACCTTACCAGAGGGCGCCCCAGCTGGCAATTCC | |
| cmR | CGATATCTGGCGAAAATGAGACGTTGATCGGCACGTAAGAGGT | 39 |
| | TCCAACTTTCACCATAATGAAATAAGATCACTACCGGGCGTATT | |
| | TTTTGAGTTATCGAGATTTTCAGGAGCTAAGGAAGCTAAAATGG | |
| | AGAAAAAAATCACTGGATATACCACCGTTGATATATCCCAATG | |
| | GCATCGTAAAGAACATTTTGAGGCATTTCAGTCAGTTGCTCAAT | |
| | GTACCTATAACCAGACCGTTCAGCTGGATATTACGGCCTTTTTA | |
| | AAGACCGTAAAGAAAAATAAGCACAAGTTTTATCCGGCCTTTAT | |
| | TCACATTCTTGCCCGCCTGATGAATGCTCATCCGGAATTCCGTA | |
| | TGGCAATGAAAGACGGTGAGCTGGTGATATGGGATAGTGTTCA | |
| | CCCTTGTTACACCGTTTTCCATGAGCAAACTGAAACGTTTTCATC | |
| | GCTCTGGAGTGAATACCACGACGATTTCCGGCAGTTTCTACACA | |
| | TATATTCGCAAGATGTGGCGTGTTACGGTGAAAACCTGGCCTAT | |
| | TTCCCTAAAGGGTTTATTGAGAATATGTTTTTCGTCTCAGCCAAT | |
| | CCCTGGGTGAGTTTCACCAGTTTTGATTTAAACGTGGCCAATAT | |
| | GGACAACTTCTTCGCCCCCGTTTTCACCATGGGCAAATATTATA | |
| | CGCAAGGCGACAAGGTGCTGATGCCGCTGGCGATTCAGGTTCA | |
| | TCATGCCGTCTGTGATGGCTTCCATGTCGGCAGAATGCTTAATG | |
| | AATTACAACAGTACTGCGATGAGTGGCAGGGCGGGGCGTAATT | |
| | TGATATCG | |
| oriV | GGGAGGGTTCGAGAAGGGGGGGCACCCCCCTTCGGCGTGCGCG | 40 |
| | GTCACGCGCACAGGGCGCAGCCCTGGTTAAAAACAAGGTTTAT | |
| | AAATATTGGTTTAAAAGCAGGTTAAAAGACAGGTTAGCGGTGG | |
| | CCGAAAAACGGGCGGAAACCCTTGCAAATGCTGGATTTTCTGCC | |
| | TGTGGACAGCCCCTCAAATGTCAATAGGTGCGCCCCTCATCTGT | |
| | CAGCACTCTGCCCCTCAAGTGTCAAGGATCGCGCCCCTCATCTG | |
| | TCAGTAGTCGCGCCCCTCAAGTGTCAATACCGCAGGGCACTTAT | |
| | CCCCAGGCTTGTCCACATCATCTGTGGGAAACTCGCGTAAAATC | |
| | AGGCGTTTTCGCCGATTTGCGAGGCTGGCCAGCTCCACGTCGCC | |
| | GGCCGAAATCGAGCCTGCCCCTCATCTGTCAACGCCGCGCCGGG | |
| | TGAGTCGGCCCCTCAAGTGTCAACGTCCGCCCCTCATCTGTCAG | |
| | TGAGGGCCAAGTTTTCCGCGAGGTATCCACAACGCCGGCGGCC | |
| | GGCCGCGGTGTCTCGCACACGGCTTCGACGGCGTTTCTGGCGCG | |
| | TTTGCAGGGCCATAGACGGCCGCCAGCCCAGCGGCGAGGGCAA | |
| | CCAG | |

TABLE 4
Fitting Parameters

| Data | ON_{Max} | n | Kₒₙ | ON_{Min} |
|---|---|---|---|---|
| FIG. 15B | 93.90 | 2.603 | 2.650 | 4.587 |
| | 90.22 | 4.245 | 11.73 | 3.208 |
| | 92.83 | 3.138 | 30.61 | 0.7300 |
| highpass | 94.29 | 2.623 | 5.328 | 1.173 |
| parameters | | | | |
| lowpass | 94.88 | 4.550 | 19.01 | 0.3330 |
| parameters | | | | |
| highpass | 91.49 | 2.519 | 4.519 | 1.457 |
| parameters | | | | |
| lowpass | 94.01 | 2.434 | 38.14 | 0.01933 |
| parameters | | | | |
| | 91.62 | −2.512 | 3.782 | 0.00889 |
| | 94.89 | 3.144 | 11.04 | 1.330 |
| | 90.89 | 2.684 | 5.545 | 4.780 |
| | 94.24 | 3.098 | 15.29 | 1.720 |
| | 91.88 | 2.900 | 41.65 | 2.727 |
| | Same as FIG. 2b | Same | Same | Same |
| | | as FIG. 2b | as FIG. 2b | as FIG. 2b |
| FIG. 10B | 28628 | 1.515 | 163.2 | 13.50 |
| FIG. 12B, black | 82.31 | 2.155 | 1.719 | 18.07 |
| FIG. 12B, gray | 93.99 | 2.669 | 2.676 | 5.613 |
| | 92.96 | −3.008 | 2.861 | 0.2667 |
| | 90.94 | 2.461 | 2.051 | 2.287 |
| | 94.75 | 2.085 | 4.983 | −0.3013 |
| | 86.67 | 2.468 | 18.80 | 2.814 |

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A biological analog signal processing circuit comprising:
(a) a first promoter operably linked to a nucleic acid encoding a regulatory protein responsive to an input signal;
(b) a second promoter responsive to the regulatory protein and operably linked to a nucleic acid encoding a first output protein, wherein activity of the second promoter is altered when bound by the regulatory protein; and,
(c) an output molecule flanked by a first set of regulatory sequences, wherein the first set of regulatory sequences interacts with the first output protein to operably link the output molecule to a third promoter.

2. A biological analog signal processing circuit comprising:
(a) a first promoter operably linked to a nucleic acid encoding a regulatory protein responsive to an input signal;
(b) a second promoter responsive to the regulatory protein and operably linked to a nucleic acid encoding a first output protein, wherein activity of the second promoter is altered when bound by the regulatory protein; and
(c) an output molecule operably linked to a third promoter, wherein the output molecule or the third promoter is flanked by a first set of regulatory sequences, wherein the first set of regulatory sequences interacts with the first output protein to unlink the output molecule from the third promoter.

3. The circuit of claim 1 further comprising:
(d) a fourth promoter responsive to the regulatory protein and operably linked to a nucleic acid encoding a second output protein, wherein activity of the fourth promoter is altered when bound by the regulatory protein; and,
(e) a second output molecule flanked by a second set of regulatory sequences, wherein the second set of regulatory sequences interacts with the second output protein to operably link the second output molecule to a fifth promoter.

4. The circuit of claim 1, wherein the promoter of (a) is a constitutively-active promoter.

5. The circuit of claim 1, wherein the regulatory protein is oxyR.

6. The circuit of claim 1, wherein the promoter of (b) and/or (d) comprises a modification that alters the binding affinity of a transcription factor or RNA polymerase for the promoter of (b) and/or (d), relative to a similar unmodified promoter.

7. The circuit of claim 6, wherein the modification is a nucleic acid mutation.

8. The circuit of claim 1, wherein (a), (b) and (c) are on a vector.

9. The circuit of claim 7, wherein (a), (b), (c) and (d) are on a vector.

10. The circuit of claim 1, wherein (a) and (b) are on a single vector.

11. The circuit of claim 10, wherein (a), (b) and (d) are on a single vector.

12. The circuit of claim 8, wherein the vector is a low copy plasmid, a medium copy plasmid or a high copy plasmid.

13. The circuit of claim 1, wherein (c) and/or (e) is on a bacterial artificial chromosome (BAC).

14. The circuit of claim 1, wherein (b) and/or (d) further comprises a sequence element that regulates production of the first output protein and is located between the second promoter and the nucleic acid encoding the first output protein.

15. The circuit of claim 14, wherein the sequence element regulates transcription or translation of the output protein.

16. The circuit of claim 14, wherein the sequence element is a ribosomal binding site.

17. The circuit of claim 16, wherein the sequence element is a modified ribosomal binding site comprising a modification that alters the binding affinity of a ribosome for the modified ribosomal binding site, relative to a similar unmodified ribosomal binding site.

18. The circuit of claim 1, wherein the promoter of (b) and/or (d) is an oxyR promoter that comprises a modification that alters the binding affinity of a transcription factor or RNA polymerase for oxyR promoter of (b), relative to a similar unmodified promoter.

19. The circuit of claim 1, wherein the first output protein of (b) is a recombinase and the first set of regulatory sequences of (c) is recombinase recognition sites.

20. The circuit of claim 1, wherein the second output protein of (d) is a recombinase and the second set of regulatory sequences of (e) is recombinase recognition sites.

21. The circuit of claim 1, wherein the first output molecule of (c) is a fluorescent protein.

22. The circuit of claim 2, wherein the second output molecule of (e) is a fluorescent protein.

23. A cell or cell lysate comprising the circuit of claim 1.

24. The cell or cell lysate of claim 23, wherein the cell is a bacterial cell.

25. The cell or cell lysate of claim 24, wherein the bacterial cell is an Escherichia coli cell.

26. The cell or cell lysate of claim 22 further comprising the input signal.

27. The cell or cell lysate of claim 26, wherein the input signal modulates activity of the regulatory protein.

28. The cell or cell lysate of claim 27, wherein the input signal activates activity of the regulatory protein.

29. The cell or cell lysate of claim 26, wherein the input signal is a chemical input signal.

30. The cell or cell lysate of claim 29, wherein the chemical input signal is hydrogen peroxide.

31. A biological bandpass filter comprising:
(a) a first promoter operably linked to a nucleic acid encoding a regulatory protein responsive to an input signal;
(b) a second promoter responsive to the regulatory protein and operably linked to a nucleic acid encoding a first bandpass protein, wherein activity of the second promoter is altered when bound by the regulatory protein;
(c) a third promoter responsive to the regulatory protein and operably linked to a nucleic acid encoding a second bandpass protein, wherein the third promoter is not the second promoter and wherein activity of the third promoter is altered when bound by the regulatory protein; and
(d) a first output molecule flanked by a first set of regulatory sequences, wherein the first set of regulatory sequences interacts with the first bandpass protein to operably link the first output molecule to a fourth promoter, wherein the fourth promoter is flanked by a second set of regulatory sequences, and wherein the second set of regulatory sequences interacts with the second bandpass protein to unlink the first output molecule from the fourth promoter.

32. A biological bandpass filter comprising:
(a) a first promoter operably linked to a nucleic acid encoding a regulatory protein responsive to an input signal;
(b) a second promoter responsive to the regulatory protein and operably linked to a nucleic acid encoding a first bandpass protein, wherein activity of the second promoter is altered when bound by the regulatory protein;
(c) a third promoter responsive to the regulatory protein and operably linked to a nucleic acid encoding a second bandpass protein, wherein the third promoter is not the second promoter and wherein activity of the third promoter is altered when bound by the regulatory protein; and
(d) a first output molecule operably linked to a fourth promoter, flanked by a first set of regulatory sequences, wherein the first set of regulatory sequences interacts with the first bandpass protein to unlink the first output molecule from the fourth promoter, wherein the first set of regulatory sequences is flanked by a second set of regulatory sequences, and wherein the second set of regulatory sequences interacts with the second bandpass protein to operably link the first output molecule to the fourth promoter.

33. The circuit of claim 31, wherein the promoter of (a) is a constitutively-active promoter.

34. The circuit of claim 31, wherein the regulatory protein is oxyR.

35. The circuit of claim 31, wherein the second promoter of (b) and/or the third promoter of (c) comprises a modification that alters the binding affinity of a transcription factor or RNA polymerase for the second promoter of (b) and or the third promoter of (c) relative to a similar unmodified promoter.

36. The circuit of claim 35, wherein the modification is a nucleic acid mutation.

37. The circuit of claim 31, wherein (a), (b) and (c) are on a vector.

38. The circuit of claim 31, wherein (a), (b) and (c) are on the same vector.

39. The circuit of claim 37, wherein the vector is a low copy plasmid, a medium copy plasmid or a high copy plasmid.

40. The circuit of claim 31, wherein (d) is on a bacterial artificial chromosome (BAC).

41. The circuit of claim 31, wherein (b) further comprises a sequence element that regulates production of the first bandpass protein and is located between the second promoter and the nucleic acid encoding the first bandpass protein.

42. The circuit of claim 31, wherein (c) further comprises a sequence element that regulates production of the second bandpass protein and is located between the third promoter and the nucleic acid encoding the second bandpass protein.

43. The circuit of claim 41, wherein the sequence element regulates transcription or translation of the first bandpass protein and/or second bandpass protein.

44. The circuit of claim 43, wherein the sequence element is a ribosomal binding site.

45. The circuit of claim 44, wherein the sequence element is a modified ribosomal binding site comprising a modification that alters the binding affinity of a ribosome for the modified ribosomal binding site, relative to a similar unmodified ribosomal binding site.

46. The circuit of claim 31, wherein the promoter of (b) is an oxyR promoter that comprises a modification that alters the binding affinity of a transcription factor or RNA polymerase for oxyR promoter of (b), relative to a similar unmodified promoter.

47. The circuit of claim 31, wherein the promoter of (c) is an oxyR promoter that comprises a modification that alters the binding affinity of a transcription factor or RNA polymerase for oxyR promoter of (c), relative to a similar unmodified promoter.

48. The circuit of claim 31, wherein the first bandpass protein of (b) is a recombinase.

49. The circuit of claim 31, wherein the second bandpass protein of (c) is a recombinase.

50. The circuit of claim 31, wherein the first set of regulatory sequences and/or the second set of regulatory sequences of (d) are recombinase recognition sites.

51. The circuit of claim 31, wherein the first output protein of (d) is a fluorescent protein.

52. A cell or cell lysate comprising the circuit claim 31.

53. The cell or cell lysate of claim 52, wherein the cell is a bacterial cell.

54. The cell or cell lysate of claim 53, wherein the bacterial cell is an Escherichia coli cell.

55. The cell or cell lysate of claim 52 further comprising the input signal.

56. The cell or cell lysate of claim 55, wherein the input signal modulates activity of the regulatory protein.

57. The cell or cell lysate of claim 56, wherein the input signal activates activity of the regulatory protein.

58. The cell or cell lysate of claim 55, wherein the input signal is a chemical input signal.

59. The cell or cell lysate of claim 58, wherein the chemical input signal is hydrogen peroxide.

60. A biological analog signal processing circuit comprising:
(a) a first promoter operably linked to a nucleic acid encoding a regulatory protein responsive to an input signal;
(b) a second promoter responsive to the regulatory protein and operably linked to a nucleic acid encoding a first bandpass protein, wherein activity of the second promoter is altered when bound by the regulatory protein;
(c) a third promoter responsive to the regulatory protein and operably linked to a nucleic acid encoding a second bandpass protein, wherein the third promoter is not the second promoter and wherein activity of the third promoter is altered when bound by the regulatory protein;
(d) a fourth promoter responsive to the regulatory protein and operably linked to a nucleic acid encoding a third bandpass protein, wherein the fourth promoter is not the first and not the second promoter and wherein activity of the fourth promoter is altered when bound by the regulatory protein;
(e) a first output molecule flanked by a first set of regulatory sequences, wherein the first set of regulatory sequences interacts with the first bandpass protein to operably link the first output molecule to a fifth promoter, wherein the fifth promoter and a sixth promoter are flanked by a second set of regulatory sequences, wherein the second set of regulatory sequences interacts with the second bandpass protein to unlink the fifth promoter from the first output molecule and to operably link the fifth promoter to a second output molecule; and,
(f) a third set of regulatory sequences flanking the sixth promoter, wherein the third set of regulatory sequences interacts with the third bandpass protein to operably link the sixth promoter to the first output molecule without unlinking the fifth promoter from the second output molecule.

61. The circuit of claim 60, wherein the promoter of (a) is a constitutively-active promoter.

62. The circuit of claim 60, wherein the regulatory protein is oxyR.

63. The circuit of claim 60, wherein the second promoter of (b) and/or the third promoter of (c) and/or the fourth promoter of (d) comprises a modification that alters the binding affinity of a transcription factor or RNA polymerase for the promoter of (b) and/or (c) and/or (d), relative to a similar unmodified promoter.

64. The circuit of claim 63, wherein the modification is a nucleic acid mutation.

65. The circuit of claim 60, wherein (a), (b), (c) and/or (d) are on a vector.

66. The circuit of claim 60, wherein (a), (b), (c) and/or (d) are on the same vector.

67. The circuit of claim 65, wherein the vector is a low copy plasmid, a medium copy plasmid or a high copy plasmid.

68. The circuit of claim 60, wherein (e) and (f) are on a bacterial artificial chromosome (BAC).

69. The circuit of claim 68, wherein (e) and (f) are on a single bacterial artificial chromosome (BAC).

70. The circuit of claim 60, wherein (b) further comprises a sequence element that regulates production of the first bandpass protein and is located between the second promoter and the nucleic acid encoding the first bandpass protein.

71. The circuit of claim 60, wherein (c) further comprises a sequence element that regulates production of the second bandpass protein and is located between the third promoter and the nucleic acid encoding the second bandpass protein.

72. The circuit of claim 60, wherein (d) further comprises a sequence element that regulates production of the third bandpass protein and is located between the fourth promoter and the nucleic acid encoding the third bandpass protein.

73. The circuit of claim 60, wherein the sequence element regulates transcription or translation of the first bandpass protein and/or the second bandpass protein and/or the third bandpass protein.

74. The circuit of claim 60, wherein the sequence element is a ribosomal binding site.

75. The circuit of claim 74, wherein the sequence element is a modified ribosomal binding site comprising a modification that alters the binding affinity of a ribosome for the modified ribosomal binding site, relative to a similar unmodified ribosomal binding site.

76. The circuit of claim 60, wherein the promoter of (b) is an oxyR promoter that comprises a modification that alters the binding affinity of a transcription factor or RNA polymerase for oxyR promoter of (b), relative to a similar unmodified promoter.

77. The circuit of claim 60, wherein the promoter of (c) is an oxyR promoter that comprises a modification that alters the binding affinity of a transcription factor for or RNA polymerase oxyR promoter of (c), relative to a similar unmodified promoter.

78. The circuit of claim 60, wherein the promoter of (d) is an oxyR promoter that comprises a modification that alters the binding affinity of a transcription factor for or RNA polymerase oxyR promoter of (d), relative to a similar unmodified promoter.

79. The circuit of claim 60, wherein the first bandpass protein of (b) is a recombinase.

80. The circuit of claim 60, wherein the second bandpass protein of (c) is a recombinase.

81. The circuit of claim 60, wherein the third bandpass protein of (d) is a recombinase.

82. The circuit of claim 60, wherein the first set of regulatory sequences and/or the second set of regulatory sequences of (d) and/or the third set of regulatory sequences of (e) are recombinase recognition sites.

83. The circuit of claim 60, wherein the first output molecule of (e) is a fluorescent protein.

84. A cell or cell lysate comprising the circuit of claim 60.

85. The cell or cell lysate of claim 84, wherein the cell is a bacterial cell.

86. The cell or cell lysate of claim 85, wherein the bacterial cell is an Escherichia coli cell.

87. The cell or cell lysate of claim 84 further comprising the input signal.

88. The cell or cell lysate of claim 87, wherein the input signal modulates activity of the regulatory protein.

89. The cell or cell lysate of claim 88, wherein the input signal activates activity of the regulatory protein.

90. The cell or cell lysate of claim 87, wherein the input signal is a chemical input signal.

91. The cell or cell lysate of claim 90, wherein the chemical input signal is hydrogen peroxide.

92. A method of analog signal processing in cells, comprising:
providing a cell or cell lysate that comprises the circuit of any one of the preceding claims; and
contacting the cell with an input signal that modulates the regulatory protein.

93. The method of claim 92 further comprising contacting the cell or cell lysate with different concentrations of the input signal.

94. The method of claim 92 further comprising detecting in the cell or cell lysate an expression level of the output molecule and, optionally, quantifying levels of the output molecule.

95. The method of claim 92, wherein the cell is a bacterial cell.

96. The method of claim 95, wherein the bacterial cell is an Escherichia coli cell.

97. The method of claim 92, wherein the output molecule is a reporter molecule, an enzyme, a therapeutic molecule or a nucleic acid molecule.
